(19) United States
(12) Patent Application Publication
(10) Pub. No.: US 2007/0011777 A1
(43) Pub. Date: Jan. 11, 2007

(54) Plants with increased activity of a class 3 branching enzyme

(76) Inventor: Claus Frohberg, Kleinmachnow (DE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/573,999

(22) PCT Filed: Sep. 29, 2004
(86) PCT No.: PCT/EP04/10984

§ 371 Date: Mar. 30, 2006

(30) Foreign Application Priority Data
Sep. 30, 2003 (EP) 03090324.9

(51) Int. Cl. A01H 1/00; C08B 31/00; C12N 15/82; C12N 5/04

(52) U.S. Cl. 800/284; 435/419; 435/468; 536/102

(57) ABSTRACT

The present invention relates to plant cells and plants, which are genetically modified wherein the genetic modification leads to an increase in the activity of a Class 3 vegetable branching enzyme in comparison with corresponding wild type plant cells or wild type plants that have not been genetically modified. Furthermore, the present invention relates to means and methods for the manufacture of such plant cells and plants. Plant cells and plants of this type synthesise a modified starch. The present invention therefore also relates to the starch synthesised by the plant cells and plants according to the invention as well as to methods for the manufacture of the starch and to the manufacture of starch derivatives of this modified starch.

The present invention relates to plant cells and plants, which are genetically modified, wherein the genetic modification leads to an increase in the activity of a Class 3 vegetable branching enzyme in comparison with corresponding wild type plant cells or wild type plants that have not been genetically modified. Furthermore, the present invention relates to means and methods for the manufacture of such plant cells and plants. Plant cells and plants of this type synthesise a modified starch. The present invention therefore also relates to the starch synthesised by the plant cells and plants according to the invention as well as to methods for the manufacture of the starch and to the manufacture of starch derivatives of this modified starch.

With regard to the increasing importance currently attributed to vegetable constituents as renewable raw material sources, one of the tasks of biotechnological research is to endeavour to adapt these vegetable raw materials to suit the requirements of the processing industry. Furthermore, in order to enable regenerating raw materials to be used in as many areas of application as possible, it is necessary to achieve a large variety of materials.

Polysaccharide starch is made up of chemically uniform base components, the glucose molecules, but constitutes a complex mixture of different molecule forms, which exhibit differences with regard to the degree of polymerisation and branching, and therefore differ strongly from one another in their physical-chemical characteristics. Discrimination is made between amylose starch, an essentially unbranched polymer made from α-1,4-glycosidically linked glucose units, and the amylopectin starch, a branched polymer, in which the branches come about by the occurrence of additional α-1,6-glycosidic links. A further essential difference between amylose and amylopectin lies in the molecular weight. While amylose, depending on the origin of the starch, has a molecular weight of 5×10⁵-10⁶ Da, that of the amylopectin lies between 10⁷ and 10⁸ Da. The two macromolecules can be differentiated by their molecular weight and their different physical-chemical characteristics, which can most easily be made visible by their different iodine bonding characteristics.

Amylose has long been looked upon as a linear polymer, consisting of α-1,4-glycosidically linked α-D-glucose monomers. In more recent studies, however, the presence of α-1,6-glycosidic branching points (ca. 0.1%) has been shown (Hizukuri and Takagi, Carbohydr. Res. 134, (1984), 1-10; Takeda et al., Carbohydr. Res. 132, (1984), 83-92).

Amylopectin constitutes a complex mixture of differently branched glucose chains. In contrast to amylose, amylopectin is more strongly branched. According to textbook information (Voet and Voet, Biochemistry, John Wiley & Sons, 1990), on average, the α-1,6 branches occur every 24 to 30 glucose residues. This is equivalent to a degree of branching of ca. 3%-4%. The figures for the degree of branching are variable and are dependent on the origin (e.g. plant species, plant type etc.) of the appropriate starch. In typical plants used for the industrial production of starch, such as maize, wheat or potato, for example, the synthesised starch consists of ca. 20%-30% amylose starch and ca. 70%-80% amylopectin starch.

The functional characteristics of the starch, along with the amylose/amylopectin ratio and the phosphate content, are strongly affected by the molecular weight, the pattern of the side chain distribution, the ion concentration, the lipid and protein content, the average grain size of the starch and the grain morphology of the starch etc. At the same time, by way of example, the solubility, the retrogradation behaviour, the water bonding capability, the film formation characteristics, the viscosity, the sticking characteristics, the freezing-thawing stability, the acid stability, the gelling strength etc. must be mentioned as important functional characteristics. The grain size of the starch can also be important for different applications.

Branching enzymes, which are also abbreviated by the designation “BE” (from Branching Enzyme; E.C. 2.4.1.18), catalyse the introduction of α-1,6 branches in α-1,4-glucans. Branching enzymes and the nucleic or amino acid sequences that characterise them are known from widely different organisms, such as bacteria, microbial fungi, mammals, algae and higher plants, for example. As only plants synthesise starch, while the above-mentioned non-vegetable organisms (e.g. bacteria, fungi and mammals) synthesise glycogen, the related branching enzymes, which are involved in the synthesis of the appropriate polymer, can also be sub-divided into glycogen branching enzymes and starch branching enzymes. Plants are therefore starch branching enzymes, which are often also referred to as Q-enzymes in older literature.

In all plant species that have been investigated up to now, the branching enzymes described can be associated with two different classes (Burton et al., 1995, Plant Journal 7, 3-15; Mizuno et al., 2001, Plant Cell Physiol. 42(4), 349-357). The association with these classes, sometimes designated in the literature with A or 2 respectively and B or 1 respectively, is based on the comparison of derived protein sequences.

As different nomenclatures have been used in the past for designating and classifying branching enzymes, Smith-White and Preiss (1994, Plant Molecular Biology Reporter 12, 67-71) (1994, Plant Molecular Biology Reporter 12, 67-71) have proposed a system for standardising this nomenclature, in which the association with the two classes of vegetable branching enzymes is also based on the comparison of derived protein sequences (Larsson et al., 1998, Plant Mol. Biol. 37, 505-511). According to this nomenclature, those vegetable branching enzymes, the amino acid sequence of which has a higher degree of identity with that of branching enzyme I of maize (GenBank Acc: D11081), is to be designated as a Class 1 branching enzyme, and those vegetable branching enzymes, the coding amino acid sequence of which has a higher degree of identity with that of branching enzyme II of maize (GenBank Acc: AF072725), is to be designated as a Class 2 branching enzyme. The designation of gene products, which are coding for branching enzymes, are, in accordance with the nomenclature of Smith-White and Preiss, to be incorporated in the already existing nomenclature by means of E.C. numbers. This results in the so-called GPN (Gene Product Number) Codes for the two classes, namely GPN 2.2.4.1.18:1 for Class 1 branching enzymes and GPN 2.2.4.18:2 for Class 2 branching enzymes.

The following vegetable or starch branching enzymes therefore belong to Class 1 (GPN 2.2.1.18:1) according to the nomenclature proposed by Smith-White and Preiss (1994, Plant Molecular Biology Reporter 12, 67-71):

BE I from Aegilops tauschii (GenBank Acc: AF525746), BE I from barley (GenBank Acc: AY304541), BE from tapioca (GenBank Acc: X77012), BE I (frequently also described as BE 1) from rice (GenBank Acc: D11082, D10752, D10838), BE 3 from bean (GenBank Acc: AB029549), BE II from pea (GenBank Acc: X80010), BE from millet (GenBank Acc: AF169833), BE I from potato (GenBank Acc: Y08786, X69805), BE from wheat (GenBank Acc: Y12320, AF076679, AF002820) and BE I from maize (GenBank Acc: D11081, AAO20100, E03435, AY176762, U17897, AF072724).

At the same time, the amino acid sequences for different Class 1 branching enzymes each have an identity of more than 60% with the amino acid sequence of branching enzyme I from maize (GenBank Acc: D11081).

Branching enzymes, which belong to Class 2 (GPN 2.2.1.18:2) according to the nomenclature proposed by Smith-White and Preiss (1994, Plant Molecular Biology Reporter 12, 67-71) are, for example, BE IIa from Aegilops tauschii (GenBank Acc: AF338431, WO 9914314), BE2-1 and BE2-2 from Arabidosis thaliana (BE2-1 GenBank Acc: NM_{—}129196 CAA04134; BE2-2 GenBank Acc: CAB82930, NM_{—}120446), BE IIa and BE IIb from barley (BE IIa GenBank Acc: AF064560; BE IIb GenBank Acc: AF064561), BE II from sweet potato (GenBank Acc: AB071286), BE III and BE IV (frequently also described as BE 3 or BE 4 respectively) from rice (BE III GenBank Acc: D16201; BE IV GenBank Acc: AB023498), BE 1 from bean (GenBank Acc: AB029548), BE I from pea (GenBank Acc: X80009), BE IIb from millet (GenBank Acc: AY304540), BE II from potato (GenBank Acc: AJ000004, AJ011885, AJ011888, AJ011889, AJ011890), BE II or BE IIa from wheat (GenBank Acc: Y11282, AF286319, AF338432, U66376) and BE II, or BE IIb from maize (BE II GenBank Acc: AAA18571, T02981; BE IIb GenBank Acc: AF072725, L08065). At the same time, the amino acid sequences for different Class 2 branching enzymes each have an identity of more than 60% with the amino acid sequence of branching enzyme IIb from maize (GenBank Acc: AF072725).

Vegetable or starch branching enzymes belong to the family of alpha-amylolytic enzymes (Svensson, 1994, Plant Molecular Biology 25, 141-157; Jespersen et al., 1991, Biochem J. 280, 51-55) and, with regard to the amino acid sequence, have four conserved domains (Baba et al., 1991, Biochem. Biophys. Res. Commun. 181(1), 87-94; Kuriki et al., 1996, J. of Protein Chemistry 15(3), 305-313).

Structural predictions based on mathematical calculations derived from experimental data such as protein crystal structures (Pfam: http://hits.isb-sib.ch/cgi-bin/PFSCAN?) show that all previously known branching enzymes from higher plants have two domains: an alpha-amylase domain and an iso-amylase domain. Here, the iso-amylase domain lies closer to the N-terminus of the protein than the alpha-amylase domain.

Plants are known, for example, which have a reduced activity of a Class 2 branching enzyme due to a mutation. These include the so-called “amylose extender” (ae) mutants from maize (Stindard et al., 1993, Plant Cell 5, 1555-1566; Boyer and Preiss, 1978, Biochem. Biophys. Res. Commun. 80, 169-175) and rice (Mizuno et al., 1993, J. Biol. Chem. 268, 19084-19091), as well as the “rugosus” (r) mutation in pea (Smith, 1988, Planta 175, 270-279; Bhattacharyya et al., 1990, Cell 60, 115-122). All these mutants are distinguished by the fact that they synthesise a starch, which has an increased amylose content in comparison with starches from corresponding plants, which do not have this mutation.

Furthermore, genetically modified potato plants are described, in which the activity of a BE I (Class 1) branching enzyme (Kossmann et al., 1991, Mol Gen Genet 230, 39-44; Safford et al., 1998, Carbohydrate Polymers 35, 155-168), or the activity of a BEII (Class 2) branching enzyme (Jobling et al., 1999, The Plant Journal 18), or the activity of a BEI and BEII branching enzyme (Schwall et al., 2000, Nature Biotechnology 18, 551-554, Jobling et al., 2003, Nature Biotechnology 21, 77-80) are reduced.

Previously, it has been possible to associate all vegetable branching enzymes to one or both of the classes described above. Plant cells or plants, which have an increased activity of a branching enzyme, which cannot be associated with these classes, are unknown.

The object of the present invention is therefore based on providing modified starches, new plant cells and/or plants, which synthesise such a modified starch, as well as methods for producing said plants.

This problem is solved by the embodiments described in the claims.

The present invention therefore relates to genetically modified plant cells and plants, characterised in that the plant cells or plants have an increased activity of at least one Class 3 branching enzyme in comparison with corresponding wild type plant cells or wild type plants that have not been genetically modified.

A first aspect of the present invention relates to a plant cell or plant, which is genetically modified, wherein the genetic modification leads to an increase in the activity of at least one Class 3 branching enzyme in comparison with corresponding wild type plant cells or wild type plants that have not been genetically modified.

At the same time, the genetic modification can be any genetic modification, which leads to an increase in the activity of at least one Class 3 branching enzyme in comparison with corresponding wild type plant cells or wild type plants that have not been genetically modified.

In conjunction with the present invention, the term “wild type plant cell” means that the plant cells concerned were used as starting material for the manufacture of the plant cells according to the invention, i.e. their genetic information, apart from the introduced genetic modification, corresponds to that of a plant cell according to the invention.

In conjunction with the present invention, the term “wild type plant” means that the plants concerned were used as starting material for the manufacture of the plants according to the invention, i.e. their genetic information, apart from the introduced genetic modification, corresponds to that of a plant according to the invention.

In conjunction with the present invention, the term “corresponding” means that, in the comparison of several objects, the objects concerned that are compared with one another have been kept under the same conditions. In conjunction with the present invention, the term “corresponding” in conjunction with wild type plant cell or wild type plant means that the plant cells or plants, which are compared with one another, have been raised under the same cultivation conditions and that they have the same (cultivation) age.

The plant cells according to the invention have an increased activity of at least one Class 3 branching enzyme in comparison with corresponding wild type plant cells that have not been genetically modified.

Here, within the framework of the present invention, the term “increased activity” means an increase in the expression of endogenous genes, which code Class 3 branching enzymes and/or an increase in the quantity of Class 3 branching enzyme protein in the cells and/or an increase in the enzymatic activity of Class 3 branching enzymes in the cells.

The increase in the expression can, for example, be determined by measuring the quantity of transcripts coding Class 3 branching enzyme, e.g. using Northern blot analysis or RT-PCR. Here, an increase preferably means an increase in the amount of transcripts in comparison with corresponding cells that have not been genetically modified by at least 50%, in particular by at least 70%, preferably by at least 85% and particularly preferably by at least 100%.

The increase in the amount of protein of a Class 3 branching enzyme, which results in an increased activity of this protein in the plant cells concerned, can, for example, be determined by immunological methods such as Western blot analysis, ELISA (Enzyme Linked Immuno Sorbent Assay) or RIA (Radio Immune Assay). Here, an increase preferably means an increase in the amount of Class 3 branching enzyme protein in comparison with corresponding cells that have not been genetically modified by at least 50%, in particular by at least 70%, preferably by at least 85% and particularly preferably by at least 100%.

Within the framework of the present invention, the term “branching enzyme” (α-1,4-glucan: α-1,4-glucan 6-glycosyltransferase, E.C. 2.4.1.18) is understood to mean a protein, which catalyses a transglycosylation reaction, in which α-1,4 links of an α-1,4-glucan donor are hydrolysed and the thereby released α-1,4-glucan chains are transferred to an α-1,4-glucan acceptor chain and, in doing so, are transformed into α-1,6-links. In particular, within the framework of the present invention, the term “branching enzyme” is to be understood to mean a vegetable branching enzyme, i.e. a starch branching enzyme.

The activity of a branching enzyme can be demonstrated, for example, with the help of native acrylamide gel electrophoresis. In doing so, proteins are first separated electrophoretically and, after incubation in buffers containing an activity, which synthesises linear α-1,4-glucan chains (e.g. starch phosphorylase a) and its substrate (e.g. glucose-6-phosphate), the corresponding gels are coloured with iodine (Kimihiko et al., 1980, Analytical Biochemistry 108, 16-24).

Furthermore, branching enzymes in microbial organisms, such as the E. coli strain KV832 for example (Kiel et al., 1987 Mol. Gen. Genet 207: 294-301), which do not synthesise branched α-glucans, can be expressed. If an activity of a branching enzyme is introduced into the microbial organism due to the expression of a foreign gene in such strains (e.g. E. coli KV832), then the branching enzyme activity can be demonstrated by treating colonies of these organisms with iodine vapour, for example. Colonies, which synthesise linear α-1,4-glucans, turn blue in this test, while colonies, which synthesise branched glucans by expressing an additional enzymatic activity of a branching enzyme, turn reddish-brown after treating with iodine vapour. It is also possible to express proteins in phosphoglucomutase mutants of E. coli to identify a branching enzyme activity of appropriate proteins (Buettcher et al., 1999, Biochem. Biophys. Acta 1432, 406-412).

A further possibility of demonstrating branching enzyme activity of proteins is the use of a reaction stimulated by phosphorylase a and the subsequent separation of the products by means of thin film chromatography (Almstrupp et al., 2000, Analytical Biochemistry 286, 297-300).

Branching enzyme activities can also be demonstrated with the help of the methods described in Guan and Preiss (1993, Plant Physiol. 102. 1269-1273) and Kuriki et al. (1996, J. of Protein Chemistry 15, 305-313).

In conjunction with the present invention, the term “Class 3 branching enzyme” is to be understood as a branching enzyme, which has a higher degree of identity with the amino acid sequence specified in Seq ID NO 4 than with that of the branching enzyme BE I from maize (GenBank Acc: D11081) or with that of the branching enzyme BE IIb from maize (GenBank Acc: AF072725). Preferably, the Class 3 branching enzyme comes from starch-storing plants, particularly preferably from plant species of the genus Solanum, especially preferably from Solanum tuberosum.

In a further embodiment of the present invention, amino acid sequences coding Class 3 branching enzymes have an identity of at least 60% with the sequence specified in SEQ ID NO 4, in particular of at least 70%, preferably of at least 80% and particularly preferably of at least 90% and especially preferably of at least 95%.

According to the invention, Class 3 branching enzymes have an iso-amylase domain (Pfam acc.: Pf02922) and an alpha-amylase domain (Pfam acc: Pf00128). According to the invention, the iso-amylase domain and the alpha-amylase domain in amino acid sequences coding branching enzymes are separated from one another by the presence of further amino acids, which do not belong to these two domains.

Class 3 branching enzymes according to the invention are distinguished by the fact that the iso-amylase domain is separated from the alpha-amylase domain by a greater number of amino acids than the iso-amylase domain and the alpha-amylase domain of Class 1 and 2 branching enzymes.

Class 3 branching enzymes according to the invention are preferably distinguished with regard to their amino acid sequence by the fact that they have at least 70, preferably at least 100, particularly preferably at least 130 and especially preferably at least 198 amino acids between the iso-amylase domain and the alpha-amylase domain. In a further embodiment of the present invention, in the case of an amino acid sequence coding a Class 3 branching enzyme, the C-terminal end of the iso-amylase domain is separated from the N-terminal beginning of the alpha-amylase domain by 70 to 198, preferably by 100 to 198, particularly preferably by 130 to 198 and especially particularly preferably by 150 to 198 amino acids.

With the help of the Pfam database (Batemann et al., 2002, Nucleic Acids Research 30, 276-280; accessible via http://www.sanger.ac.uk/Software/Pfam/, http://www.cgb.ki.se/Pfam/; http://pfam.jouy.inra.fr/ or http://pfam.wustl.edu/), it is possible for the person skilled in the art to determine whether amino acid sequences already have known domains (e.g. an iso-amylase domain and/or an alpha-amylase domain).

Pfam is a database put together by experts, which classifies amino acid sequences into so-called families. Here, the assignment of an amino acid sequence to a family is carried out on the basis of so-called domains, which are to be looked upon as functional and structural components of proteins. A domain is defined as a structural unit or a repeatedly occurring amino acid sequence unit, which can occur in proteins with widely different functions. Along with information relating to the amino acid sequence of known proteins, further knowledge (e.g. evidence of the enzymatic activity, crystal structure data) is also used for the assignment of a protein to a family. Each family is assigned a name and an “accession” number (e.g. Name: Isoamylase_N, acc: PF02922). A constituent part of each family in the Pfam database is, amongst other things, a so-called “seed alignment”. The “seed alignment” contains the amino acid sequences of representative proteins of a family. Starting from “seed alignments”, a so-called profile HMM (“profile Hidden Markov Model”; overview article in: Durbin et al., “Biological Sequence Analysis: Probabilistic Models of Proteins and Nucleic Acids”, Cambridge University Press, 1998, ISBN 0-521-62041-4) is produced using the HMMER 2 software (freely available under http://hmmer.wustl.edu/). The HMMs produced have names and are stored in the Pfam database specifically for the correspondingly assigned domains. In contrast to classical, multiple “alignments” (e.g. produced using the Clustal W program or the Blossum62 algorithm), HMMs are based on a valid statistical theory (Bayes theory of conditional probability, Markoff chains) and enable an interrogation sequence (query) to be assigned to a family based on the use of position-specific evaluation matrices. This enables an assignment to be made even when there are considerable differences in the amino acid sequences between the interrogation sequence (Query) and a comparison sequence (e.g. amino acid sequence entry in a database).

The domain structure of the amino acid sequence concerned can be determined by means of a comparison of the HMMs stored in the Pfam database with amino acid sequences, which are entered as a so-called interrogation sequence (query) (e.g. under http://hits.isb-sib.ch/cgi-bin/PFSCAN?).

In conjunction with the present invention, the term “iso-amylase domain” is to be understood to mean a Pfam iso-amylase domain (acc: Pf02922). At the same time, the HMM describing this Pfam iso-amylase domain is to be produced with the HMMER 2 [2.3.1] software, starting from a “seed alignment”, which contains the amino acid sequences shown in Table 1. In conjunction with the present invention, the “seed alignment” is produced by means of the Clustal W program (Thompson et al., Nucleic Acids Research 22 (1994), 4673-4680; see below). The following settings must be chosen to produce the appropriate HMMs: Build Method of HMM: hmmbuild-F HMM_Is, hmmcalibrate-seed 0 HMM_Is; Gathering cutoff: 2.3 2.3; Trusted cutoff: 2.3 2.2; Noise cutoff: 2.1 2.1). Further information for producing the HMM of the Pfam iso-amylase domain (acc: Pf02922) is given in Table 3.

In conjunction with the present invention, the term “alpha-amylase domain” is to be understood to mean a Pfam alpha-amylase domain (acc: Pf00128). At the same time, the HMM describing this Pfam alpha-amylase domain is to be produced with the HMMER 2 [2.3.1] software, starting from a “seed alignment”, which contains the amino acid sequences shown in Table 2. Here, the “seed alignment” is produced by means of HMM_simulated_annealing (http://www.psc.edu/general/software/packages/hmmer/manual/node11.html#SECTION00321000000000000000). The following settings must be chosen to produce the appropriate HMM: Build Method of HMM: hmmbuild-F HMM_Is, hmmcalibrate-seed 0 HMM_Is; Gathering cutoff: −82.0-82.0; Trusted cutoff: −81.7-81.7; Noise cutoff: −82.7-82.7). Further information for producing the HMM of the Pfam alpha-amylase domain (acc: Pf00128) is given in Table 4.

In conjunction with the present invention, the term “Class 3 branching enzyme gene” is to be understood to mean a nucleic acid molecule (cDNA, DNA), which codes a Class 3 branching enzyme, preferably a Class 3 branching enzyme from starch-storing plants, particularly preferably from plant species of the genus Solanum, especially preferably from Solanum tuberosum.

A preferred embodiment of the present invention relates to a genetically modified plant cell according to the invention or a genetically modified plant according to the invention, wherein the genetic modification consists in the introduction of at least one foreign nucleic acid molecule into the genome of the plant cell or into the genome of the plant.

In this context, the term “genetic modification” means the introduction of homologous and/or heterologous foreign nucleic acid molecules into the genome of a plant cell or into the genome of a plant, wherein said introduction of these molecules leads to an increase in the activity of a Class 3 branching enzyme.

The plant cells according to the invention or plants according to the invention are modified with regard to their genetic information by the introduction of a foreign nucleic acid molecule. The presence or the expression of the foreign nucleic acid molecule leads to a phenotypic change. Here, “phenotypic” change means preferably a measurable change of one or more functions of the cells. For example, the genetically modified plant cells according to the invention and the genetically modified plants according to the invention exhibit an increase in the activity of a Class 3 branching enzyme due to the presence or on the expression of the introduced nucleic add molecule.

In conjunction with the present invention, the term “foreign nucleic acid molecule” is understood to mean such a molecule that either does not occur naturally in the corresponding wild type plant cells, or that does not occur naturally in the concrete spatial arrangement in wild type plant cells, or that is localised at a place in the genome of the wild type plant cell at which it does not occur naturally. Preferably, the foreign nucleic acid molecule is a recombinant molecule, which consists of different elements, the combination or specific spatial arrangement of which does not occur naturally in vegetable cells.

In principle, the foreign nucleic acid molecule can be any nucleic acid molecule, which effects an increase in the activity of a Class 3 branching enzyme in the plant cell or plant.

In conjunction with the present invention, the term “genome” is to be understood to mean the totality of the genetic material present in a vegetable cell. It is known to the person skilled in the art that, as well as the cell nucleus, other compartments (e.g. plastids, mitochondrions) also contain genetic material.

In a further embodiment, the plant cells according to the invention and the plants according to the invention are characterised in that the foreign nucleic acid molecule codes a Class 3 branching enzyme, preferably a Class 3 branching enzyme from starch-storing plants, particularly preferably from plants of a species of the genus Solanum, especially preferably from Solanum tuberosum.

In a particularly preferred embodiment, the foreign nucleic acid molecule codes a Class 3 branching enzyme with the amino acid sequence specified in SEQ ID NO 4.

A large number of techniques are available for the introduction of DNA into a vegetable host cell. These techniques include the transformation of vegetable cells with T-DNA using Agrobacterium tumefaciens or Agrobacterium rhizogenes as the transformation medium, the fusion of protoplasts, injection, the electroporation of DNA, the introduction of DNA by means of the biolistic approach as well as other possibilities.

The use of agrobacteria-mediated transformation of plant cells has been intensively investigated and adequately described in EP 120516; Hoekema, IN: The Binary Plant Vector System Offsetdrukkerij Kanters B. V., Alblasserdam (1985), Chapter V; Fraley et al., Crit. Rev. Plant Sci. 4, 1-46 and by An et al. EMBO J. 4, (1985), 277-287. For the transformation of potato, see Rocha-Sosa et al., EMBO J. 8, (1989), 29-33, for example.

The transformation of monocotyledonous plants by means of vectors based on agrobacterium transformation has also been described (Chan et al., Plant Mol. Biol. 22, (1993), 491-506; Hiei et al., Plant J. 6, (1994) 271-282; Deng et al, Science in China 33, (1990), 28-34; Wilmink et al., Plant Cell Reports 11, (1992), 76-80; May et al., Bio/Technology 13, (1995), 486-492; Conner and Domisse, Int. J. Plant Sci. 153 (1992), 550-555; Ritchie et al, Transgenic Res. 2, (1993), 252-265). An alternative system to the transformation of monocotyledonous plants is transformation by means of the biolistic approach (Wan and Lemaux, Plant Physiol. 104, (1994), 37-48; Vasil et al., Bio/Technology 11 (1993), 1553-1558; Ritala et al., Plant Mol. Biol. 24, (1994), 317-325; Spencer et al., Theor. Appl. Genet. 79, (1990), 625-631), protoplast transformation, electroporation of partially permeabilised cells and the introduction of DNA by means of glass fibres. In particular, the transformation of maize has been described in the literature many times (cf. e.g. WO95106128, EP0513849, EP0465875, EP0292435; Fromm et al., Biotechnology 8, (1990), 833-844; Gordon-Kamm et al., Plant Cell 2, (1990), 603-618; Koziel et al., Biotechnology 11 (1993), 194-200; Moroc et al., Theor. Appl. Genet. 80, (1990), 721-726).

The successful transformation of other types of cereal has also already been described, for example for barley (Wan and Lemaux, see above; Ritala et al., see above; Krens et al., Nature 296, (1982), 72-74) and for wheat (Nehra et al., Plant J. 5, (1994), 285-297). All the above methods are suitable within the framework of the present invention.

Amongst other things, the plant cells according to the invention and the plants according to the invention can be differentiated from wild type plant cells and wild type plants respectively in that they contain a foreign nucleic acid molecule, which does not occur naturally in wild type plant cells or wild type plants, or in that such a molecule is present integrated at a place in the genome of the plant cell according to the invention or in the genome of the plant according to the invention at which it does not occur in wild type plant cells or wild type plants, i.e. in a different genomic environment. Furthermore, plant cells according to the invention and plants according to the invention of this type differ from wild type plant cells and wild type plants respectively in that they contain at least one copy of the foreign nucleic acid molecule stably integrated within their genome, possibly in addition to naturally occurring copies of such a molecule in the wild type plant cells or wild type plants. If the foreign nucleic acid molecule(s) introduced into the plant cells according to the invention or into the plants according to the invention is (are) additional copies of molecules already occurring naturally in the wild type plant cells or wild type plants respectively, then the plant cells according to the invention and the plants according to the invention can be differentiated from wild type plant cells or wild type plants respectively in particular in that this additional copy or these additional copies is (are) localised at places in the genome at which it does not occur (or they do not occur) in wild type plant cells or wild type plants. This can be verified, for example, with the help of a Southern Blot Analysis.

Furthermore, the plant cells according to the invention and the plants according to the invention can preferably be differentiated from wild type plant cells or wild type plants respectively by at least one of the following characteristics: If the foreign nucleic acid module that has been introduced is heterologous with respect to the plant cell or plant, then the plant cells according to the invention or plants according to the invention have transcripts of the introduced nucleic acid molecules. These can be verified, for example, by Northern blot analysis or by RT-PCR (Reverse Transcription Polymerase Chain Reaction). Plant cells according to the invention and plants according to the invention, which express an antisense and/or an RNAi transcript, can be verified, for example, with the help of specific nucleic acid probes, which are complimentary to the RNA (occurring naturally in the plant cell), which is coding for the protein. Preferably, the plant cells according to the invention and the plants according to the invention contain a protein, which is coded by an introduced nucleic acid molecule. This can be demonstrated by immunological methods, for example, in particular by a Western Blot Analysis.

If the foreign nucleic acid module that has been introduced is homologous with respect to the plant cell or plant, the plant cells according to the invention or plants according to the invention can be differentiated from wild type plant cells or wild type plants respectively due to the additional expression of the introduced foreign nucleic acid molecule, for example. The plant cells according to the invention and the plants according to the invention preferably contain (sense and/or antisense) transcripts of the foreign nucleic acid molecules. This can be demonstrated by Northern blot analysis, for example, or with the help of so-called quantitative PCR.

In a special embodiment, the plant cells according to the invention and the plants according to the invention are transgenic plant cells or transgenic plants respectively

In a further embodiment, the present invention relates to plant cells according to the invention and plants according to the invention wherein the foreign nucleic acid molecule is chosen from the group consisting of
a) Nucleic acid molecules, which code a protein with the amino acid sequence given under Seq ID NO 4;
b) Nucleic acid molecules, which code a protein, the amino acid sequence of which has an identity of at least 50% with the amino acid sequence given under SEQ ID NO: 4;
c) Nucleic acid molecules, which include the nucleotide sequence shown under Seq ID NO 3 or a complimentary sequence;
d) Nucleic acid molecules, the nucleic acid sequence of which has an identity of at least 50% with the nucleic acid sequences described under a) or c);
e) Nucleic acid molecules, which hybridise with at least one strand of the nucleic acid molecules described under a) or c) under stringent conditions;
f) Nucleic acid molecules, the nucleotide sequence of which deviates from the sequence of the nucleic acid molecules identified under a), b), c), d), e) or f) due to the degeneration of the genetic code; and
g) Nucleic acid molecules, which represent fragments, allelic variants and/or derivatives of the nucleic acid molecules identified under a), b), c), d), e) or f).

The amino acid sequence specified in SEQ ID NO 4 codes a protein with the activity of a Class 3 branching enzyme from Solanum tuberosum.

A plasmid was deposited with the Deutsche Sammlung von Mikroorganismen und Zellkulturen GmbH, Mascheroder Weg 1b, 38124 Braunschweig, Germany, in accordance with the Budapest Treaty on 15 Sep. 2003 under the number DSM 15926. The amino acid sequence shown SEQ ID NO 4 can be derived from the coding region of the cDNA sequence integrated in plasmid DSM 15926 and codes for a Class 3 branching enzyme from Solanum tuberosum. The present invention relates to nucleic acid molecules, which code a protein with the enzymatic activity of a Class 3 branching enzyme, which includes the amino acid sequence, which is coded by the insertion in plasmid DSM 15926, wherein the coded. protein has an identity of at least 70%, preferably of at least 80%, particularly preferably of at least 90% and especially preferably of 95% with the amino acid sequence, which can be derived from the insertion coding a Class 3 branching enzyme in DSM 15926.

The proteins coded from the different varieties of nucleic acid molecules according to the invention have certain common characteristics. These can include, for example, biological activity, molecular weight, immunological reactivity, conformation, the presence of structural and/or functional domains etc, as well as physical characteristics such as, for example, the running behaviour in gel electrophoresis, chromatographic behaviour, sedimentation coefficients, solubility, spectroscopic characteristics, stability; optimum pH, optimum temperature etc.

The molecular weight of the Class 3 branching enzyme from Solanum tuberosum derived from the amino acid sequence shown under SEQ ID NO 4 is ca. 103 kDa. The derived molecular weight of a protein according to the invention therefore preferably lies in the range from 85 kDa to 120 kDa, preferably in the range from 95 kDa to 110 kDa and particularly preferably from ca. kDa 100 to 105 kDa.

The nucleic acid sequence shown in SEQ ID NO 3 is a cDNA sequence, which includes the coding region for a Class 3 branching enzyme from Solanum tuberosum.

The present invention therefore also relates to nucleic acid molecules, which code a Class 3 branching enzyme and the coding region of the nucleotide sequence shown under Seq ID NO 3 or a complimentary sequence, nucleic acid molecules, which include the coding region of the nucleotide sequence of the insertion contained in plasmid DSM 15926, and nucleic acid molecules, which have an identity of at least 70%, preferably of at least 80%, particularly preferably of at least 90% and especially preferably of at least 95% with the said nucleic acid molecules.

With the help of the sequence information of the nucleic acid molecule according to the invention or with the help of the nucleic acid molecule according to the invention, it is now possible for the person skilled in the art to isolate homologous sequences from other plant species, preferably from starch-storing plants, preferably from plant species of the genus Solanum, particularly preferably from Solanum tuberosum. This can be carried out, for example, with the help of conventional methods such as the examination of cDNA or genomic banks with suitable hybridisation samples. The person skilled in the art knows that homologous sequences can also be isolated with the help of (degenerated) oligonucleotides and the use of PCR-based methods.

The examination of databases, such as are made available, for example, by EMBL (http://www.ebi.ac.uk/Tools/index.htm) or NCBI (National Center for Biotechnology Information, http://www.ncbi.nlm.nih.gov/), can also be used for identifying homologous sequences, which code for a Class 3 branching enzyme. In this case, one or more sequences are specified as a so-called query. This query sequence is then compared by means of statistical computer programs with sequences, which are contained in the selected databases. Such database queries (e.g. blast or fasta searches) are known to the person skilled in the art and can be carried out by various providers.

If such a database query is carried out, e.g. at the NCBI (National Center for Biotechnology Information, http://www.ncbi.nlm.nih.gov/), then the standard settings, which are specified for the particular comparison inquiry, should be used. For protein sequence comparisons (blastp), these are the following settings: Limit entrez=not activated; Filter=low complexity activated; Expect value=10; word size=3; Matrix=BLOSUM62; Gap costs: Existence=11, Extension=1.

For nucleic acid sequence comparisons (blastn), the following parameters must be set: Limit entrez=not activated; Filter=low complexity activated; Expect value=10; word size=11.

With such a database search, the sequences described in the present invention can be used as a query sequence in order to identify further nucleic acid molecules and/or proteins, which code a Class 3 branching enzyme.

With the help of the described methods, it is also possible to identify and/or isolate nucleic acid molecules according to the invention, which hybridise with the sequence specified under SEQ ID NO: 3 and which code a Class 3 branching enzyme.

Within the framework of the present invention, the term “hybridising” means hybridisation under conventional hybridisation conditions, preferably under stringent conditions such as, for example, are described in Sambrock et al., Molecular Cloning, A Laboratory Manual, 2nd Ed. (1989) Cold Spring Harbor Laboratory Press, Cold Spring Harbor, N.Y.). Particularly preferably, “hybridising” means hybridisation under the following conditions:
Hybridisation buffer: 2×SSC; 10× Denhardt solution (Ficoll 400+PEG+BSA; Ratio 1:1:1); 0.1% SDS; 5 mM EDTA; 50 mM Na₂HPO₄; 250 μg/ml herring sperm DNA; 50 μg/ml tRNA; or
25 M sodium phosphate buffer pH 7.2; 1 mM EDTA; 7% SDS
Hybridisation temperature: T=65 to 68° C.
Wash buffer: 0.2×SSC; 0.1% SDS
Wash temperature: T=65 to 68° C.

In principle, nucleic acid molecules, which hybridise with the nucleic acid molecules according to the invention, can originate from any plant species, which expresses an appropriate protein, preferably they originate from starch-storing plants, preferably from species of the genus Solanum, particularly preferably from Solanum tuberosum. Nucleic acid molecules, which hybridise with the molecules according to the invention, can, for example, be isolated from genomic or from cDNA libraries. The identification and isolation of nuclear acid molecules of this type can be carried out using the nucleic acid molecules according to the invention or parts of these molecules or the reverse complements of these molecules, e.g. by means of hybridisation according to standard methods (see, for example, Sambrook et al., 1989, Molecular Cloning, A Laboratory Manual, 2nd Ed. Cold Spring Harbor Laboratory Press, Cold Spring Harbor, N.Y.) or by amplification using PCR.

Nucleic acid molecules, which exactly or essentially have the nucleotide sequence specified under SEQ ID NO: 3, or parts of this sequence, can be used as hybridisation samples. The fragments used as hybridisation samples can also be synthetic fragments or oligonucleotides, which have been manufactured using established synthesising techniques and the sequence of which corresponds essentially with that of a nucleic acid molecule according to the invention. If genes have been identified and isolated, which hybridise with the nucleic acid sequences according to the invention, then a determination of this sequence and an analysis of the characteristics of the proteins coded by this sequence should be carried out in order to establish whether a Class 3 branching enzyme is involved. Homology comparisons on the level of the nucleic acid or amino acid sequence and a determination of the enzymatic activity are particularly suitable for this purpose. As described above, the activity of a Class 3 branching enzyme can take place by expression in E. coli strains, which themselves do not express an active branching enzyme (Kiel et al., 1987 Mol. Gen. Genet 207: 294-301); Guan et al., 1995, Proc. Natl. Acad. Sci. 92, 964-967).

The molecules hybridising with the nucleic acid molecules according to the invention particularly include fragments, derivatives and allelic variants of the nucleic acid molecules described above, which code a Class 3 branching enzyme from plants, preferably from starch-storing plants, preferably from plant species of the genus Solanum, particularly preferably from Solanum tuberosum. In conjunction with the present invention, the term “derivative” means that the sequences of these molecules differ at one or more positions from the sequences of the nucleic acid molecules described above and have a high degree of identity with these sequences.

Here, the deviation from the nucleic acid molecules described above can have come about, for example, due to deletion, addition, substitution, insertion or recombination.

The proteins coded from the different derivatives of nucleic acid molecules according to the invention have certain common characteristics. These can include, for example, biological activity, substrate specificity, molecular weight, immunological reactivity, conformation, the presence of structural and/or functional domains etc, as well as physical characteristics such as, for example, the running behaviour in gel electrophoresis, chromatographic behaviour, sedimentation coefficients, solubility, spectroscopic characteristics, stability; optimum pH, optimum temperature etc.

In conjunction with the present invention, the term “identity” means a sequence identity over the whole length of the coding region of at least 60%, in particular an identity of at least 70%, preferably greater than 80%, particularly preferably greater than 90% and especially of at least 95%. In conjunction with the present invention, the term “identity” is to be understood to mean the number of amino acids/nucleotides (identity) corresponding with other proteins/nucleic acids, expressed as a percentage. Identity is preferably determined by comparing the Seq. ID NO 4 or SEQ ID NO 3 with other proteins/nucleic acids with the help of computer programs. If sequences that are compared with one another have different lengths, the identity is to be determined in such a way that the number of amino acids, which have the shorter sequence in common with the longer sequence, determines the percentage quotient of the identity. Preferably, identity is determined by means of the computer program ClustalW, which is well known and available to the public (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be downloaded from different internet sites, including the IGBMC (Institut de Génétique et de Biologie Moléculaire et Cellulaire, B.P.163, 67404 Illkirch Cedex, France; ftp://ftp-igbmc.u-strasbg.fr/pub/) and the EBI (ftp://ftp.ebi.ac.uk/pub/software/) as well as from all mirrored internet sites of the EBI (European Bioinformatics Institute, Wellcome Trust Genome Campus, Hinxton, Cambridge CB10 1SD, UK).

Preferably, Version 1.8 of the ClustalW computer program is used to determine the identity between proteins according to the invention and other proteins. In doing so, the following parameters must be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Preferably, Version 1.8 of the ClustalW computer program is used to determine the identity between the nucleotide sequence of the nucleic acid molecules according to the invention, for example, and the nucleotide sequence of other nucleic acid molecules. In doing so, the following parameters must be set:
KTUPLE=2, TOPDIAGS=4, PAIRGAP=5, DNAMATRIX:IUB, GAPOPEN=10, GAPEXT=5, MAXDIV=40, TRANSITIONS: unweighted.

Furthermore, identity means that functional and/or structural equivalence exists between the nucleic acid molecules concerned or the proteins coded by them. The nucleic acid molecules, which are homologous to the molecules described above and constitute derivatives of these molecules, are generally variations of these molecules, which constitute modifications, which execute the same biological function. At the same time, the variations can occur naturally, for example they can be sequences from other plant species, or they can be mutations, wherein these mutations may have occurred in a natural manner or have been introduced by objective mutagenesis. The variations can also be synthetically manufactured sequences. The allelic variants can be both naturally occurring variants and also synthetically manufactured variants or variants produced by recombinant DNA techniques. Nucleic acid molecules, which deviate from nucleic acid molecules according to the invention due to degeneration of the genetic code, constitute a special form of derivatives.

The nucleic acid molecules according to the invention can be any nucleic acid molecules, in particular DNA or RNA molecules, for example cDNA, genomic DNA, mRNA etc. They can be naturally occurring molecules or molecules manufactured by genetic or chemical synthesis methods. They can be single-stranded molecules, which either contain the coding or the non-coding strand, or double-stranded molecules.

Furthermore, the present invention relates to nucleic acid molecules of at least 21, preferably more than 50 and particularly preferably more than 200 nucleotides length, which specifically hybridise with at least one nucleic acid molecule according to the invention. Here, specifically hybridise means that these molecules hybridise with nucleic acid molecules, which code a protein according to the invention, but not with nucleic acid molecules, which code other proteins.

A further embodiment of the present invention relates to plant cells according to the invention and plants according to the invention wherein the foreign nucleic acid molecule is chosen from the group consisting of
a) T-DNA molecules, which lead to an increase in the expression of a Class 3 branching enzyme gene due to integration in the vegetable genome (T-DNA activation tagging);
b) DNA molecules, which contain transposons, which lead to an increase in the expression of a Class 3 branching enzyme gene due to integration in the vegetable genome (transposon activation tagging);
c) DNA molecules, which code a Class 3 branching enzyme and which are linked with regulatory sequences, which guarantee transcription in vegetable cells and lead to an increase in Class 3 branching enzyme activity in the cell.
d) Nucleic acid molecules introduced by means of in vivo mutagenesis, which lead to a mutation or an insertion of a heterologous sequence in at least one endogenous gene coding a Class 3 branching enzyme, wherein the mutation or insertion effects an increase in the expression of a gene coding a Class 3 branching enzyme.

In conjunction with the present invention, plant cells according to the invention and plants according to the invention can also be manufactured by the use of so-called insertion mutagenesis (overview article: Thorneycroft et al., 2001, Journal of experimental Botany 52 (361), 1593-1601). In conjunction with the present invention, insertion mutagenesis is to be understood to mean particularly the insertion of transposons or so-called transfer DNA (T-DNA) into a gene or in the vicinity of a gene coding for a Class 3 branching enzyme, whereby as a result of which the activity of a Class 3 branching enzyme in the cell concerned is increased.

The transposons can be both those that occur naturally in the cell (endogenous transposons) and also those that do not occur naturally in said cell but are introduced into the cell (heterologous transposons) by means of genetic engineering methods, such as transformation of the cell, for example. Changing the expression of genes by means of transposons is known to the person skilled in the art. An overview of the use of endogenous and heterologous transposons as tools in plant biotechnology is presented in Ramachandran and Sundaresan (2001, Plant Physiology and Biochemistry 39, 234-252).

T-DNA insertion mutagenesis is based on the fact that certain sections (T-DNA) of Ti plasmids from Agrobacterium can integrate into the genome of vegetable cells. The place of integration in the vegetable chromosome is not defined, but can take place at any point. If the T-DNA integrates into a part of the chromosome or in the vicinity of a part of the chromosome, which constitutes a gene function, then this can lead to an increase in the gene expression and thus also to a change in the activity of a protein coded by the gene concerned.

Here, the sequences inserted into the genome (in particular transposons or T-DNA) are distinguished by the fact that they contain sequences, which lead to an activation of regulatory sequences of a Class 3 branching enzyme gene (“activation tagging”).

Plant cells and plants according to the invention can be produced by means of the so-called “activation tagging” method (see, for example, Walden et al., Plant J. (1991), 281-288; Walden et al., Plant Mol. Biol. 26 (1994), 1521-1528). These methods are based on activating endogenous promoters by means of “enhancer” sequences, such as the enhancer of the 35S RNA promoter of the cauliflower mosaic virus, or the octopine synthase enhancer.

In conjunction with the present invention, the term “T-DNA activation tagging” is to be understood to mean a T-DNA fragment, which contains “enhancer” sequences and which leads to an increase in the activity of at least one Class 3 branching enzyme by integration into the genome of a plant cell.

In conjunction with the present invention, the term “transposon activation tagging” is to be understood to mean a transposon, which contains “enhancer” sequences and which leads to an increase in the activity of at least one Class 3 branching enzyme by integration into the genome of a plant cell.

In a preferred embodiment, the DNA molecules according to the invention, which code a Class 3 branching enzyme, are linked with regulatory sequences, which guarantee transcription in vegetable cells and lead to an increase in Class 3 branching enzyme activity in the cell. In this case, the nucleic acid molecules according to the invention are present in “sense” orientation to the regulatory sequences.

For expressing nucleic acid molecules according to the invention, which code Class 3 branching enzymes, these are preferably linked with regulatory DNA sequences, which guarantee transcription in vegetable cells. In particular, these include promoters. In general, any promoter that is active in vegetable cells is eligible for expression.

At the same time, the promoter can be chosen so that expression takes place constitutively or only in a certain tissue, at a certain stage of the plant development or at a time determined by external influences. The promoter can be homologous or heterologous both with respect to the plant and with respect to the nucleic acid molecule.

Suitable promoters are, for example, the promoter of the 35S RNA of the cauliflower mosaic virus and the ubiquitin promoter from maize for constitutive expression, the patatin promoter B33 (Rocha-Sosa et al., EMBO J. 8 (1989), 23-29) for tuber-specific expression in potatoes or a promoter, which only ensures expression in photosynthetically active tissues, e.g. the ST-LS1 promoter (Stockhaus et al., Proc. Natl. Acad. Sci. USA 84 (1987), 7943-7947; Stockhaus et al., EMBO J. 8 (1989), 2445-2451) or, for endosperm-specific expression of the HMG promoter from wheat, the USP promoter, the phaseolin promoter, promoters of zein genes from maize (Pedersen et al., Cell 29 (1982), 1015-1026; Quatroccio et al., Plant Mol. Biol. 15 (1990), 81-93), glutelin promoter (Leisy et al., Plant Mol. Biol. 14 (1990), 41-50; Zheng et al., Plant J. 4 (1993), 357-366; Yoshihara et al., FEBS Lett. 383 (1996), 213-218) or shrunken-1 promoter (Werr et al., EMBO J. 4 (1985), 1373-1380). However, promoters can also be used, which are only activated at a time determined by external influences (see for example WO 9307279). Promoters of heat-shock proteins, which allow simple induction, can be of particular interest here. Furthermore, seed-specific promoters can be used, such as the USP promoter from Vicia faba, which guarantees seed-specific expression in Vicia faba and other plants (Fiedler et al., Plant Mol. Biol. 22 (1993), 669-679; Bäumlein et al., Mol. Gen. Genet. 225 (1991), 459-467).

Furthermore, a termination sequence (polyadenylation signal) can be present, which is used for adding a poly-A tail to the transcript. A function in the stabilisation of the transcripts is ascribed to the poly-A tail. Elements of this type are described in the literature (cf. Gielen et al., EMBO J. 8 (1989), 23-29) and can be exchanged at will.

Furthermore, plant cells according to the invention and plants according to the invention can be manufactured by means of so-called “in situ activation”. In this case, the introduced genetic modification effects a change in the regulatory sequences of endogenous Class 3 branching enzyme genes, which leads to an increased expression of Class 3 branching enzyme genes. Preferably, the activation of a Class 3 branching enzyme gene takes place by “in vivo” mutagenesis of a promoter or of “enhancer” sequences of an endogenous Class 3 branching enzyme gene. In doing so, a promoter or an “enhancer” sequence, for example, can be changed in such a way that the mutation produced leads to an increased expression of a Class 3 branching enzyme gene in plant cells according to the invention or plants according to the invention in comparison with the expression of a Class 3 branching enzyme gene in wild type plant cells or wild type plants. The mutation in a promoter or an “enhancer” sequence can also lead to Class 3 branching enzyme genes in plant cells according to the invention or plants according to the invention being expressed at a time at which they would not be expressed in wild type plant cells or wild type plants.

In so-called “in vivo mutagenesis”, a hybrid RNA-DNA oligonucleotide (“Chimeroplast”) is introduced into plant cells (Kipp, P. B. et al., Poster Session at the “5^{th} International Congress of Plant Molecular Biology, 21-27 Sep. 1997, Singapore; R. A. Dixon and C. J. Arntzen, meeting report on “Metabolic Engineering in Transgenic Plants”, Keystone Symposia, Copper Mountain, Colo., USA, TIBTECH 15, (1997), 441-447; international patent application WO 9515972; Kren et al., Hepatology 25, (1997), 1462-1468; Cole-Strauss et al., Science 273, (1996), 1386-1389; Beetham et al., 1999, PNAS 96, 8774-8778).

A part of the DNA components of the RNA-DNA oligonucleotide is homologous to a nucleic acid sequence of an endogenous Class 3 branching enzyme gene, but, in comparison with the nucleic acid sequence of a Class 3 branching enzyme gene, it has a mutation or contains a heterologous region, which is surrounded by the homologous regions.

By base pairing of the homologous regions of the RNA-DNA oligonucleotide and the endogenous nucleic acid molecule followed by homologous recombination, the mutation or heterologous region contained in the DNA components of the RNA-DNA oligonucleotide can be transferred into the genome of a plant cell. This leads to an increase in the activity of one or more Class 3 branching enzymes.

All these methods are based on the introduction of a foreign nucleic acid molecule into the genome of a plant cell or plant and are therefore basically suitable for the manufacture of plant cells according to the invention and plants according to the invention.

Surprisingly, it has been found that plant cells according to the invention and plants according to the invention synthesise a modified starch in comparison with starch of corresponding wild type plant cells or wild type plants that have not been genetically modified.

The plant cells according to the invention and plants according to the invention synthesise a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesised starch in wild type plant cells or wild type plants, so that this is better suited for special applications.

The present invention therefore also includes plant cells according to the invention and plants according to the invention, which synthesise a modified starch.

In conjunction with the present invention, the term “modified starch” means that the starch has changed physical-chemical characteristics compared with non-modified starch obtainable from corresponding wild type plant cells or wild type plants.

In a preferred embodiment of the present invention, the modified starch is native starch.

In conjunction with the present invention, the term “native starch” means that the starch is isolated from plants according to the invention, harvestable plant plants according to the invention or propagation material of plants according to the invention by methods known to the person skilled in the art.

Furthermore, genetically modified plants, which contain the plant cells according to the invention, are also the subject matter of the invention. Plants of this type can be produced from plant cells according to the invention by regeneration.

In principle, the plants according to the invention can be plants of any plant species, i.e. both monocotyledonous and dicotyledonous plants. Preferably they are useful plants, i.e. plants, which are cultivated by people for the purposes of food or for technical, in particular industrial purposes.

In a further preferred embodiment, the plant according to the invention is a starch-storing plant.

In a further preferred embodiment, the present invention relates to starch-storing plants according to the invention of the genus Solanum, in particular Solanum tuberosum.

The term “starch-storing plants” includes all plants with starch-storing plant parts such as, for example, maize, rice, wheat, rye, oat, barley, cassava, potato, sago, mung bean, pea or sorghum. Preferred starch-storing plant parts are, for example, tubers, storage roots and grains containing an endosperm; tubers are particularly preferred; tubers of potato plants are especially preferred.

In conjunction with the present invention, the term “potato plant” or “potato” means plant species of the genus Solanum, in particular tuber-producing species of the genus Solanum and especially Solanum tuberosum.

The present invention also relates to propagation material of plants according to the invention containing a plant cell according to the invention.

Here, the term “propagation material” includes those constituents of the plant that are suitable for producing offspring by vegetative or sexual means. Cuttings, callus cultures, rhizomes or tubers, for example, are suitable for vegetative propagation. Other propagation material includes, for example, fruits, seeds, seedlings, protoplasts, cell cultures, etc. Preferably, the propagation material is seeds and particularly preferably tubers.

In a further embodiment, the present invention relates to harvestable plant parts of plants according to the invention such as fruits, storage roots, roots, blooms, buds, shoots or stems, preferably seeds or tubers, wherein these harvestable parts contain plant cells according to the invention.

Furthermore, the present invention also relates to a method for the manufacture of a genetically modified plant according to the invention, wherein
a) a plant cell is genetically modified, whereby the genetic modification leads to an increase in the activity of a Class 3 vegetable branching enzyme in comparison with corresponding wild type plant cells that have not been genetically modified;
b) a plant is regenerated from plant cells from Step a); and
c) if necessary, further plants are produced with the help of the plants according to Step b).

The genetic modification introduced into the plant cell according to Step a) can basically be any type of modification, which leads to the reduction [sic?] of the activity of a Class 3 branching enzyme.

The regeneration of the plants according to Step (b) can be carried out using methods known to the person skilled in the art (e.g. described in “Plant Cell Culture Protocols”, 1999, edt. by R. D. Hall, Humana Press, ISBN 0-89603-549-2).

The production of further plants according to Step (c) of the method according to the invention can be carried out, for example, by vegetative propagation (for example using cuttings, tubers or by means of callus culture and regeneration of whole plants) or by sexual propagation. Here, sexual propagation preferably takes place under controlled conditions, i.e. selected plants with particular characteristics are crossed and propagated with one another.

In a preferred embodiment of the method according to the invention, the genetic modification consists in the introduction of a foreign nucleic acid molecule into the genome of the plant cell, wherein the presence or the expression of said foreign nucleic acid molecule leads to an increased activity of a Class 3 branching enzyme in the cell.

In a further preferred embodiment, the method according to the invention is used for producing potato plants according to the invention.

In a further preferred embodiment of the method according to the invention, the foreign nucleic acid molecule is chosen from the group consisting of
a) Nucleic acid molecules, which code a protein with the amino acid sequence given under Seq ID NO 4;
b) Nucleic acid molecules, which code a protein, the amino acid sequence of which has an identity of at least 50% with the amino acid sequence given under SEQ ID NO: 4;
c) Nucleic acid molecules, which include the nucleotide sequence shown under Seq ID NO 3 or a complimentary sequence;
d) Nucleic acid molecules, the nucleic acid sequence of which has an identity of at least 50% with the nucleic acid sequences described under a) or c);
e) Nucleic acid molecules, which hybridise with at least one strand of the nucleic acid molecules described under a) or c) under stringent conditions;
f) Nucleic acid molecules, the nucleotide sequence of which deviates from the sequence of the nucleic acid molecules identified under a), b), c), d), e) or f) due to the degeneration of the genetic code; and
g) Nucleic acid molecules, which represent fragments, allelic variants and/or derivatives of the nucleic acid molecules identified under a), b), c), d), e) or f).

In a further preferred embodiment of the method according to the invention, the foreign nucleic acid molecule is chosen from the group consisting of
a) T-DNA molecules, which lead to an increase in the expression of a Class 3 branching enzyme gene due to integration in the vegetable genome (T-DNA activation tagging);
b) DNA molecules, which contain transposons, which lead to an increase in the expression of a Class 3 branching enzyme gene due to integration in the vegetable genome (transposon activation tagging);
c) DNA molecules, which code a Class 3 branching enzyme and which are linked with regulatory sequences, which guarantee transcription in vegetable cells and lead to an increase in Class 3 branching enzyme activity in the cell;
d) Nucleic acid molecules introduced by means of in vivo mutagenesis, which lead to a mutation or an insertion of a heterologous sequence in at least one endogenous gene coding a Class 3 branching enzyme, wherein the mutation or insertion effects an increase in the expression of a gene coding a Class 3 branching enzyme.

The present invention also relates to the plants obtained by the method according to the invention.

In a further embodiment of the method according to the invention, the plants according to the invention synthesise a modified starch in comparison with corresponding wild type plants that have not been genetically modified.

Surprisingly, it has been found that plant cells and plants, which have an increased activity of a Class 3 branching enzyme, synthesise a modified starch.

The present invention also relates to the plants obtained by the method according to the invention.

The present invention also relates to modified starches obtainable from plant cells according to the invention or plants according to the invention, from propagation material according to the invention or from harvestable plant parts according to the invention. In a particularly preferred embodiment, the present invention relates to modified potato starch.

Furthermore the present invention relates to a method for the manufacture of a modified starch including the step of extracting the starch from a plant cell according to the invention or from a plant according to the invention, from propagation material according to the invention of such a plant and/or from harvestable plant parts according to the invention of such a plant, preferably from starch-storing parts according to the invention of a plant. Preferably, such a method also includes the step of harvesting the cultivated plants or plant parts and/or the propagation material of these plants before the extraction of the starch and, further, particularly preferably the step of cultivating plants according to the invention before harvesting.

Methods for extracting starches from plants or from starch-storing parts of plants are known to the person skilled in the art. Furthermore, methods for extracting starch from different starch-storing plants are described, e.g. in Starch: Chemistry and Technology (Publisher: Whistler, BeMiller and Paschall (1994), 2nd Edition, Academic Press Inc. London Ltd; ISBN 0-12-746270-8; see e.g. Chapter XII, Page 412-468: Maize and Sorghum Starches: Manufacture; by Watson; Chapter XIII, Page 469-479: Tapioca, Arrowroot and Sago Starches: Manufacture; by Corbishley and Miller; Chapter XIV, Page 479-490: Potato starch: Manufacture and Uses; by Mitch; Chapter XV, Page 491 to 506: Wheat starch: Manufacture, Modification and Uses; by Knight and Oson; and Chapter XVI, Page 507 to 528: Rice starch: Manufacture and Uses; by Rohmer and Klem; Maize starch: Eckhoff et al., Cereal Chem. 73 (1996), 54-57, the extraction of maize starch on an industrial scale is generally achieved by so-called “wet milling”.). Devices, which are in common use in methods for extracting starch from plant material are separators, decanters, hydrocyclones, spray dryers and fluid bed dryers.

In conjunction with the present invention, the term “starch-storing parts” is to be understood to mean such parts of a plant in which, in contrast to transitory leaf starch, starch is stored as a deposit for surviving for longer periods. Preferred starch-storing parts are tubers, storage roots, seeds or endosperm; particularly preferred are potato tubers or the endospermo of maize, wheat or rice plants.

Modified starch obtainable by a method according to the invention for manufacturing modified starch is also the subject matter of the present invention.

Furthermore, the use of plant cells according to the invention or plants according to the invention for manufacturing a modified starch are the subject matter of the present invention.

The person skilled in the art knows that the characteristics of starch can be changed by thermal, chemical, enzymatic or mechanical derivation, for example. Derived starches are particularly suitable for different applications in the foodstuffs and/or non-foodstuffs sector. The starches according to the invention are better suited as a starting substance for the manufacture of derived starches than conventional starches.

The present invention therefore also relates to the manufacture of a derived starch, wherein modified starch according to the invention is derived retrospectively.

In conjunction with the present invention, the term “derived starch” is to be understood to mean a modified starch according to the invention, the characteristics of which have been changed after isolation from vegetable cells with the help of chemical, enzymatic, thermal or mechanical methods.

In a preferred embodiment of the present invention, the derived starch according to the invention is starch that has been heat-treated and/or acid-treated.

In a further preferred embodiment, the derived starches are starch ethers, in particular starch alkyl ethers, O-allyl ethers, hydroxylalkyl ethers, O-carboxylmethyl ethers, nitrogen-containing starch ethers, phosphate-containing starch ethers or sulphur-containing starch ethers.

In a further preferred embodiment, the derived starches are cross-linked starches.

In a further preferred embodiment, the derived starches are starch graft polymers.

In a further preferred embodiment, the derived starches are oxidised starches.

In a further preferred embodiment, the derived starches are starch esters, in particular starch esters, which have been introduced into the starch using organic acids. Particularly preferably these are phosphate, nitrate, sulphate, xanthate, acetate or citrate starches.

The derived starches according to the invention are suitable for different applications in the foodstuffs and/or non-foodstuffs sector. Methods for manufacturing derived starches according to the invention are known to the person skilled in the art and are adequately described in the general literature. An overview on the manufacture of derived starches can be found, for example, in Orthoefer (in Corn, Chemistry and Technology, 1987, eds. Watson und Ramstad, Chapter 16, 479-499).

Derived starch obtainable by the method according to the invention for manufacturing a derived starch is also the subject matter of the present invention.

Furthermore, the use of modified starches according to the invention for manufacturing derived starch is the subject matter of the present invention.

### DESCRIPTION OF SEQUENCES

SEQ ID NO 1: Nucleic acid sequence containing the coding region of the 3′-area of a Class 3 branching enzyme from Solanum tuberosum (cv Désirée). This sequence is inserted in plasmid AN 46-196.

SEQ ID NO 2: Nucleic acid sequence containing the coding region of the 5′-area of a Class 3 branching enzyme from Solanum tuberosum (cv Désirée). This sequence is inserted in plasmid AN 47-196.

SEQ ID NO 3: Nucleic acid sequence containing the full coding region of a Class 3 branching enzyme from Solanum tuberosum (cv Désirée). This sequence is inserted in plasmid AN 49 and was deposited with the Deutsche Sammlung von Mikroorganismen und Zellkulturen GmbH, Mascheroder Weg 1b, 38124 Braunschweig, Germany, in accordance with the Budapest Treaty on 15 Sep. 2003 under the number DSM 15926.

SEQ ID NO 4: Amino acid sequence coding a Class 3 branching enzyme from Solanum tuberosum (cv Désirée). This sequence can be derived from the nucleic acid sequence inserted in plasmid AN 49 or from the nucleic acid sequence described under SEQ ID NO 3.

SEQ ID NO 5: Nucleic acid sequence containing the full coding region of a Class 3 branching enzyme from Solanum tuberosum (cv Désirée). This sequence has been obtained by combining the nucleic acid sequences described under SEQ ID NO 1 and SEQ ID NO 2. This nucleic acid sequence constitutes an allelic variant of the nucleic acid sequence described under SEQ ID NO 3 coding a Class 3 branching enzyme.

SEQ ID NO 6: Amino acid sequence coding a Class 3 branching enzyme from Solanum tuberosum (cv Désirée). This sequence can be derived from the nucleic acid sequence described under SEQ ID NO 5 and constitutes an allelic variant of the amino acid sequence described under SEQ ID NO 4 coding a Class 3 branching enzyme.

### DESCRIPTION OF THE FIGURES

FIG. 1 Shows the relative amount of mRNA in seeds of genetically modified rice plants (GOAS0453), expressing a potato branching enzyme Class 3. Also shoen is the example of a genetically non modified wild type plant (wt).

### GENERAL METHODS

The following methods were used in the examples:

1. Demonstration of the Activity of a Class 3 Branching Enzyme

The activity of a Class 3 branching enzyme was demonstrated with the help of non-denaturing gel electrophoresis as follows:

To isolate proteins from plants, the test material was ground with a pestle in liquid nitrogen, absorbed into an extraction buffer (50 mM Na citrate, pH 6.5; 1 mM EDTA, 4 mM DTT) and, after centrifugation (10 min, 14.000 g, 4° C.), was used directly for measurement of the protein content according to Bradford. Subsequently, 5 μg to 20 μg total protein extract was mixed with 4× loading buffer (20% glycerol, 125 mM Tris HCl, pH 6.8) and loaded onto a BE activity gel. The BE activity gel was made up as follows: 2.5 ml 30% acrylamide:0.8% bisacrylamide, 0.1 ml running buffer, 7.4 ml H₂O, 10% ammonium persulphate solution and 5 μl N,N,N′,N′-tetramethylethylenediamine (TEMED). The running buffer (RB) was made up as follows: RB=30.2 g Tris base, pH 8.0, 144 g glycine on 1 L H₂O. On completion of the gel run, each of the gels was incubated overnight at 37° C. in 25 ml “phosphorylase buffer” (25 ml 1M Na citrate pH 7.0, 0.47 g glucose-1-phosphate, 12.5 mg AMP, 2.5 mg phosphorylase a/b from “rabbit”). The gels were coloured with Lugol's solution.

2. Transformation of Oryza Sativa (cv. M202)

Rice plants were transformed in accordance with the methods described by Hiei et al. (1994, Plant Journal 6(2), 271-282).

3. Starch Analysis

a) Determination of the Amylose Content and of the Amylose/Amylopectin Ratio

Rice flour was obtained from polished rice by milling the grains in a Cyclotec labmill to particle size smaller than 0.5 mm.

Amylose content was determined by the method of Juliano.(1971) (Cereal Science Today, 16, p 334-340) with slight modifications. 50 mg of rice flour are weighed in a 100 ml Erlenmeyer flask and wetted with 1 ml of 95% ethanol to avoid lumping. After addition of 9 ml of 1 M NaOH the sample is heated to 100° C. for 20 min, cooled to room temperature and filled up to 100 ml with destined water.

An aliquot of 100 μl is mixed with 10 μl 1M acetic acid, 20 μl Lugol solution (0.2% I₂ and 2% KI) and 970 μl water. After 10 minutes the OD at 620 nm is determined. The amylose content is calculated from a standard curve generated from samples with defined amount of potato amylose.

b) Determination of the Phosphate Content

In starch, the positions C2, C3 and C6 of the glucose units can be phosphorylated. Determination of C6-P content of rice starch is performed either on single mature grains hydrolysed in 200 μl of 0.7 M HCl or on 50 mg rice flour hydroylsed in 500 μl 0.7 M HCl. After 4 h hydrolysis at 95° C. under continuous shaking, samples are centrifuged for 10 minutes at 15500×g and the supernatants are applied to a spin module containing 0.2 μm PTFE-filter membran for complete clarification of the hydrolyzate (5 min 1000×g). 20 μl of the supernatants are mixed with 180 μl of imidazole buffer (300 mM imidazole, pH 7.2; 7.5 mM MgCl₂, 1 mM EDTA and 0.4 mM NADP). The measurement was carried out in a photometer at 340 nm. After the base absorption had been established, the enzyme reaction was started by addition of 0.4 units glucose-6-phosphate dehydrogenase (from Leuconostoc mesenteroides, Boehringer Mannheim). The change in absorption is directly proportional to the concentration of the G-6-P content of the starch.

The total phosphate content was determined by the method of Ames (Methods in Enzymology VIII, (1966), 115-118).

Approximately 50 mg of starch are treated with 30 μl of ethanolic magnesium nitrate solution and ashed for 3 hours at 500° C. in a muffle oven. The residue is treated with 300 μl of 0.5 M hydrochloric acid and incubated for 30 minutes at 60° C. One aliquot is subsequently made up to 300 μl 0.5 M hydrochloric acid and this is added to a mixture of 100 μl of 10% ascorbic acid and 600 μl of 0.42% ammonium molybdate in 2 M sulphuric acid and incubated for 20 minutes at 45° C.

This is followed by a photometric determination at 820 nm with a phosphate calibration series as standard.

c) Determination of the viscosity characteristics by means of a Rapid Visco Analyser (RVA)

3 g of rice flour (DM) are taken up in 25 ml of H₂O (VE-type water, conductivity of at least 15 mega ohm) and used for the analysis in a Rapid Visco Analyser Super3 (Newport Scientific Pty Ltd., Investmet Support Group, Warriewod NSW 2102, Australia). The apparatus is operated following the manufacturer's instructions. The viscosity values are indicated in Centipoise (cP) in accordance with the manufacturer's operating manual, which is incorporated into the description herewith by reference. To determine the viscosity of the aqueous starch solution, the starch suspension is first stirred for 10 seconds at 960 rpm and subsequently heated at 50° C. at a stirring speed of 160 rpm, initially for a minute (step 1). The temperature was then raised from 50° C. to 95° C. at a heating rate of 12° C. per minute (step 2). The temperature is held for 2.5 minutes at 95° C. (step 3) and then cooled from 95° C. to 50° C. at 12° C. per minute (step 4). In the last step (step 5), the temperature of 50° C. is held for 2 minutes. The viscosity is determined during the entire duration.

After the program has ended, the stirrer is removed and the beaker covered. The gelatinized starch is now available for the texture analysis after 24 hours incubation at room temperature.

The profile of the RVA analysis contains parameters which are shown for the comparison of different measurements and substances. In the context of the present invention, the following terms are to be understood as follows:
1. Maximum Viscosity (RVA Max)

The maximum viscosity is understood as meaning the highest viscosity value, measured in cP, obtained in step 2 or 3 of the temperature profile.
2. Minimum Viscosity (RVA Min)

The minimum viscosity is understood as meaning the lowest viscosity value, measured in cP, observed in the temperature profile after the maximum viscosity. Normally, this takes place in step 3 of the temperature profile.
3. Final Viscosity (RVA Fin)

The final viscosity is understood as meaning the viscosity value, measured in cP, observed at the end of the measurement.
4. Setback (RVA Set)

What is known as the “setback” is calculated by subtracting the value of the final viscosity from that of the minimum occurring after the maximum viscosity in the curve.
5. Gelatinization Temperature (RVA PT)

The gelatinization temperature is understood as meaning the point in time of the temperature profile where, for the first time, the viscosity increases drastically for a brief period.

d) Determination of the Gel Strength (Texture Analyser)

3 g of rice flour (DM) are gelatinized in the RVA apparatus in 25 ml of an aqueous suspension (temperature program: see item c) “Determination of the viscosity characteristics by means of a Rapid Visco Analyser (RVA)”) and subsequently stored for 24 hours at room temperature in a sealed container. The samples are fixed under the probe (round piston with planar surface) of a Texture Analyser TA-XT2 from Stable Micro Systems (Surrey, UK) and the gel strength was determined using the following parameters:

| | | |
|---|---|---|
| Test speed | 0.5 | mm/s |
| Depth of penetration | 7 | mm |
| Contact surface | 113 | mm² |
| Pressure | 2 | g |

e) Analysis of the Side-Chain Distribution of the Amylopectin by Means of Ion-Exchange Chromatography

To separate amylose and amylopectin, 200 mg of starch are dissolved in 50 ml reaction vessels, using 12 ml of 90% (v/v) DMSO in H₂0. After addition of 3 volumes of ethanol, the precipitate is separated by centrifugation for 10 minutes at about 1800×g at room temperature (RT). The pellet is then washed with 30 ml of ethanol, dried and dissolved in 40 ml of 1% (w/v) NaCl solution at 75° C. After the solution has cooled to 30° C., approximately 90 mg of thymol are added slowly, and this solution is incubated for at least 60 h at 30° C. The solution is then centrifuged for 30 minutes at 2000×g (RT). The supernatant is then treated with 3 volumes of ethanol, and the amylopectin which settles out is separated by centrifugation for 5 minutes at 2000×g (RT). The pellet (amylopectin) is then washed with ethanol and dried using acetone. By addition of DMSO to the pellet, one obtains a 1% solution, of which 200 μl are treated with 345 μl of water, 10 μl of 0.5 M sodium acetate (pH 3.5) and 5 μl of isoamylase (dilution 1:10; Megazyme) and incubated for about 16 hours at 37° C. A 1:5 aqueous dilution of this digest is subsequently filtered through a 0.2 μm filter, and 100 μl of the filtrate are analysed by ion exchange chromatography (HPAEC-PAD, Dionex). Separation was performed using a PA-100 column (with suitable precolumn), while detection was performed amperometrically. The elution conditions were as follows:
Solution A—0.15M NaOH

Solution B—1 M sodium acetate in 0.15M NaOH

TABLE 1
Composition of the elution buffer for the side chain analysis of the
amylopectin at different times during the HPEAC-PAD Dionex
analysis. Between the times stated, the composition of the
elution buffer changes in each case linearly.

| t (min) | Solution A (%) | Solution B (%) |
|---|---|---|
| 5 | 0 | 100 |
| 35 | 30 | 70 |
| 45 | 32 | 68 |
| 60 | 100 | 0 |
| 70 | 100 | 0 |
| 72 | 0 | 100 |
| 80 | 0 | 100 |
| Stop | | |

The determination of the relative amount of short side chains in the total of all side chains is carried out via the determination of the percentage of a particular side chain in the total of all side chains. The total of all side chains is determined via the determination of the total area under the peaks which represent the polymerization degrees of DP 6 to 34 in the HPCL chromatogram.

The percentage of a particular side chain in the total of all side chains is determined via the determination of the ratio of the area under the peak which represents this side chain in the HPLC chromatogram to the total area. The programme Chromelion 6.20 Version 6.20 from Dionex, USA, was used for determining the peak areas.

f) Determination of the Activity of the BEIII Protein

This was carried out as specified in the example.

g) DSC-Analysis (,,Differential Scanning Calorimetry”)

Investigations with the aid of DSC-analysis have been done by the method described by WO 01/19975. 10 mg starch treated with 30 μl H₂0 (VE-type water, conductivity of at least 15 mega ohm) were sealed in stainless steal pans (volume 50 μl). The pan is heated from 20° C. to 150° C. at a rate of 10° C. per minute in a Diamond DSC-instrument (Perkin Elmer). The programme Pyres from Perkin Elmer was used for determining the data.

### EXAMPLES

### Example 1

### Cloning of a Full-Length Sequence Coding a Class 3 Branching Enzyme From Solanum Tuberosum

The gene sequence coding for this Class 3 branching enzyme in Solanum tuberosum has not previously been described.

By sequence comparisons with different branching enzymes, a domain was identified, with the help of which EST databases were examined. In doing so, the EST TC73137 (TIGR database; http://www.tigr.org/tigr-scripts/tgi/tc_report.pl?tc=TC73137&species=potato) from potato was identified.

With the help of the primers B1_Asp (GAT GGG TAC CAG CAC TTC TAC TTG GCA GAG G) and B2_Sal (TCA AGT CGA CCA CAA CCA GTC CAT TTC TGG), a sequence from a tuber-specific cDNA bank from Solanum tuberosum (cv. Désirée) corresponding to this EST sequence was amplified. Attempts to use leaf-specific, “sink”-tissue-specific or “source”-tissue-specific cDNA banks as a template for the PCR reaction led to no amplification.

In order to amplify the whole coding sequence of the branching enzyme concerned, which up to now had also included unknown sequences, primers were manufactured, which were complimentary to the ends of the previously known sequence and vector sequences of the cDNA banks concerned. With all the primer combinations for the amplification of a full-length sequence of a Class 3 branching enzyme used in this approach, it was not possible to amplify any further area. Hereupon, EST databases of tomato were examined again.

In this case, two ESTs from tomato were identified (TIGR database; BG127920 and TC130382), which either had a high homology to the amplification of the Class 3 branching enzyme from potato described above (TC130382) and (BG127920) respectively, or to the putative branching enzyme gene from arabidopsis (GenBank: GP|9294564|dbj|BABO2827.1).

Primers were now manufactured again in order to also amplify previously unknown sequences of the Class 3 branching enzyme. By means of PCR, the 3′-area of the Class 3 branching enzyme was amplified from a cDNA bank, made from tubers of Solanum tuberosum (cv. Désirée), with the primers KM2_Spe (5′-TCAAACTAGTCACAACCAGTCCATTTCTGG-3′) and So_putE (5′-CACTTTAGAAGGTATCAGAGC-3′). The fragment with a size of ca. 1 kb that was obtained was cloned undirectedly in the pCR4-TOPO vector from Invitrogen (product number: 45-0030). The plasmid produced was designated as AN 46-196. The sequence of the inserted fragments in the plasmid AN 46-196 is shown under SEQ ID NO 1.

The 5′-area was likewise amplified by means of PCR technology and using the primers So_put5′ (5′-GTATTTCTGCGAAGGAACGACC-3′) and So_putA (5′-AACAATGCTCTCTCTGTCGG-3′) from the same cDNA bank. The fragment with a size of ca. 2 kb that was obtained was cloned undirectedly in the pCR4-TOPO vector from Invitrogen (product number: 45-0030). The plasmid produced was designated as AN 47-196. The sequence of the inserted fragments in the plasmid AN 47-196 is shown under SEQ ID NO 2.

Primers were now manufactured again in order to amplify a full-length sequence. The following primers were used: SO_putA (AACAATGCTCTCTCTGTCGG) and SO_putE (CACTTTAGAAGGTATCAGAGC). A PCR product with an approximate size of 3.2 kb was obtained and was cloned in the pCR2.1 vector from Invitrogen (product number: 45-0030). The plasmid obtained (filed under DSM 15926) was designated as AN 49. The sequence of the inserted fragments in the plasmid AN 49 is shown under SEQ ID NO3.

### Example 2

### Information on Vectors and Plasmids

Manufacture of the Expression Vector ME5/6

pGSV71 is a derivative of the plasmid pGSV7, which derives from the intermediate vector pGSV1. pGSV1 constitutes a derivative of pGSC1700, the construction of which has been described by Cornelissen and Vanderwiele (Nucleic Acid Research 17, (1989), 19-25). pGSV1 was obtained from pGSC1700 by deletion of the carbenicillin resistance gene and deletion of the T-DNA sequences of the TL-DNA region of the plasmid pTiB6S3.

pGSV7 contains the replication origin of the plasmid pBR322 (Bolivar et al., Gene 2, (1977), 95-113) as well as the replication origin of the Pseudomonas plasmid pVS1 (Itoh et al., Plasmid 11, (1984), 206). pGSV7 also contains the selectable marker gene aadA, from the transposon Tn1331 from Klebsiella pneumoniae, which gives resistance against the antibiotics spectinomycin and streptomycin (Tolmasky, Plasmid 24 (3), (1990), 218-226; Tolmasky and Crosa, Plasmid 29(1), (1993), 31-40).

The plasmid pGSV71 was obtained by cloning a chimeric bar gene between the border regions of pGSV7. The chimeric bar gene contains the promoter sequence of the cauliflower mosaic virus for initiating the transcription (Odell et al., Nature 313, (1985), 180), the bar gene from Streptomyces hygroscopicus (Thompson et al., Embo J. 6, (1987), 2519-2523) and the 3′-untranslated area of the nopaline synthase gene of the T-DNA of pTiT37 for terminating the transcription and polyadenylation. The bar gene provides tolerance against the herbicide glufosinate ammonium.

At position 198-222, the T-DNA is given the right edge sequence of the TL-DNA from the plasmid pTiB6S3 (Gielen et al., EMBO J. 3, (1984), 835-846). A polylinker sequence is located between nucleotide 223-249. The nucleotides 250-1634 contain the P35S3 promoter region of the cauliflower mosaic virus (Odell et al., see above). The coding sequence of the phosphinothricin resistance gene (bar) from Streptomyces hygroscopicus (Thompson et al. 1987, see above) is contained between the nucleotides 1635-2186. At the same time, the two end codons at the 5′-end of the bar wild type gene were replaced by the codons ATG and GAC. A polylinker sequence is located between the nucleotides 2187-2205. The 260-bp-long Taql fragment of the untranslated 3′-end of the nopaline synthase gene (3′nos) from the T-DNA of the plasmid pTiT37 (Depicker et al., J. Mol. Appl. Genet. 1, (1982), 561-573) is located between the nucleotides 2206 and 2465. The nucleotides 2466-2519 contain a polylinker sequence. The left edge sequence of the TL-DNA from pTiB6S3 (Gielen et al., EMBO J. 3, (1984), 835-846) is located between the nucleotides 2520-2544.

The sector pGSV71 was then cut and smoothed with the enzyme Pstl. The B33 promoter and the ocs cassette were cut from the vector pB33-Kan as an EcoRI-HindIII fragment and smoothed and inserted in the vector pGSV71, which had been cut and smoothed with Pstl. The vector obtained was used as the starting vector for the construction of ME5/6. An oligonucleotide containing the sites EcoRI, Pacl, Spel, Srfl, Spel, Notl, Pacl and EcoRI was introduced into the Pstl site situated between the B33 promoter and ocs element while doubling the Pstl site. The expression vector obtained was designated as ME5/6.

Manufacture of the Expression Vector IL103-123

In the further course of events, a BamHI fragment of ME5-6 was replaced by a PCR product that had been extended by a few sites but was otherwise identical, resulting in the plasmid UL1-17. The B33 promoter contained in UL1-17 was cut out with the restriction enzymes HindIII and Pstl and the vector relegated after smoothing the ends, producing the vector ML18-56. This vector was opened with Munl and Pstl and an MCS synthesised by means of two oligonuclotides with appropriately sticky ends was added. The resulting plasmid ML72-123 was opened with the restriction enzymes Hpal and Munl and oligonucleotides for detection sequences of further restriction enzymes introduced (IR96-123). Subsequently, an Ecl136II/EcoRV PCR product for the globulin promoter for rice was spliced into the EcoRV site of IR96-123, as a result of which the base vector for an endosperm-specific expression of genes of different origin was produced. The vector is designated in the following as IR103-123.

Manufacture of the Plasmid AH33-191

The manufacture of the plasmid AH33-191 for an endosperm-specific expression of a potential Class 3 branching enzyme from potato in rice was carried out by splicing an EcoRV/Sacl fragment of the appropriate cDNA from the plasmid AN49 into the appropriate sites of IR103-123.

### Example 3

### Genetically Modified Plants With Increased Class 3 Branching Enzyme Activity

In order to produce transgenic rice plants, which have an increased expression of a Class 3 branching enzyme gene, the T-DNA of the plasmid AH33-191 was transferred into rice plants (M202) with the help of agrobacteria, as described above under General Methods. Plants obtained by the transformation with the plasmid AH33-191 were designated GAOS0453.

Analysis of the transgenic rice plants obtained, with the help of non-denaturising gel electrophoresis of protein extracts from grains of wild type plant cells and/or protein extracts from genetically modified plants, showed that the grains of genetically modified plant cells have an increased activity of a Class 3 branching enzyme in comparison with protein extracts from grains of wild type plant cells.

In addition the expression level of potato branching enzyme Class 3 in transgenic rice plants was also evaluated by analysis of the amount of mRNA by means of quantitative PCR (Q-PCR).

RNA was extracted from single immature seeds (10 to 12 days after pollination). Immature seeds were frozen in liquid nitrogen and homogenized in a ball mill (MM200, Retsch, Germany, at 30 Hz, 45 seconds, 4 mm ball). Afterwards RNA was isolated by use of the SV 96 total RNA Isolation System (Promega, Prd. No. Z3500 according to manufacturers specifications (protocol No. 294) before it was treated with 10 μl each of RQ1 RNase-Free-DNase (Promega, Prd. No. M6101) according to manufacturers specifications. Equal amounts of RNA isolated from four single seeds harvested from one single plant was pooled before analysis by quantitative PCR.

Quantitative PCR analysis was carried out by use of the Access RT-PCR System (Promega, Prd. No. A1260) according to the manufacturers specifications. Under the used conditions the following amplification primers were hybridizing specifically to RNA of potato starch branching enzyme Class 3: St_BE-f2 (TCA GGT CTA CAA GTT GAC CCG A ) and _BE-r2 (GTA GAA CCT TCC CTT TTG TGT GA). For monitoring expression levels the primer St_BE-Fam(2) labelled with two fluorescent dyes (FAM-CAT GAT CAC TCT AGC AAT CAA AGT GCC-TAMRA) was used.

For calculation of relative expression levels an endogenous mRNA present in all plants, coding S-adenosylmethionindecarboxylase was amplified in parallel by using the following amplification primers: S-AMD-F1 (GGC TTC GAC GCC TCT ACT CT) AND S-AMD-R2 (AAG GGC CAA AGC ACC TGA G). The primer FAM-S-AMD labelled with two fluorescent dyes (FAM-ACC CTC TTC ACC AGG TCG CCA-TAMRA) was included into each of the reactions.

The Q-PCR reaction was carried out each in a total volume of 30 μl comprising 350 ng RNA extracted from plants, AMV/Tfl reaction buffer (provided with Promega Prd. No. A1260), 3 mM MgSO₄, 500 nM of the respective primers, 0.2 mM of each desoxyribonucleotide, 0.6 μl reverse transcriptase, 0.6 μl Tfl Polymerase (according to Promega's protocol 294) and in addition 150 nM fluorescent primer ST_BE-Fam(2) or 100 nM of fluorescent primer FAM-S_AMD, respectively. Control reactions performed in parallel did not contain reverse transcriptase.

Reaction conditions for Q-RT-PCR:

| | | |
|---|---|---|
| Step 1: | 55° C. | 30 minutes |
| Step 2: | 94° C. | 2 minutes |
| Step 3: | 94° C. | 15 seconds |
| Step 4: | 60° C. | 1 minute |

40 repeats were performed between step 3 and step 4.

The fluorescent signal was detected during step 4 with ABI Prism 7700 (Applied Biosystems) equipment.

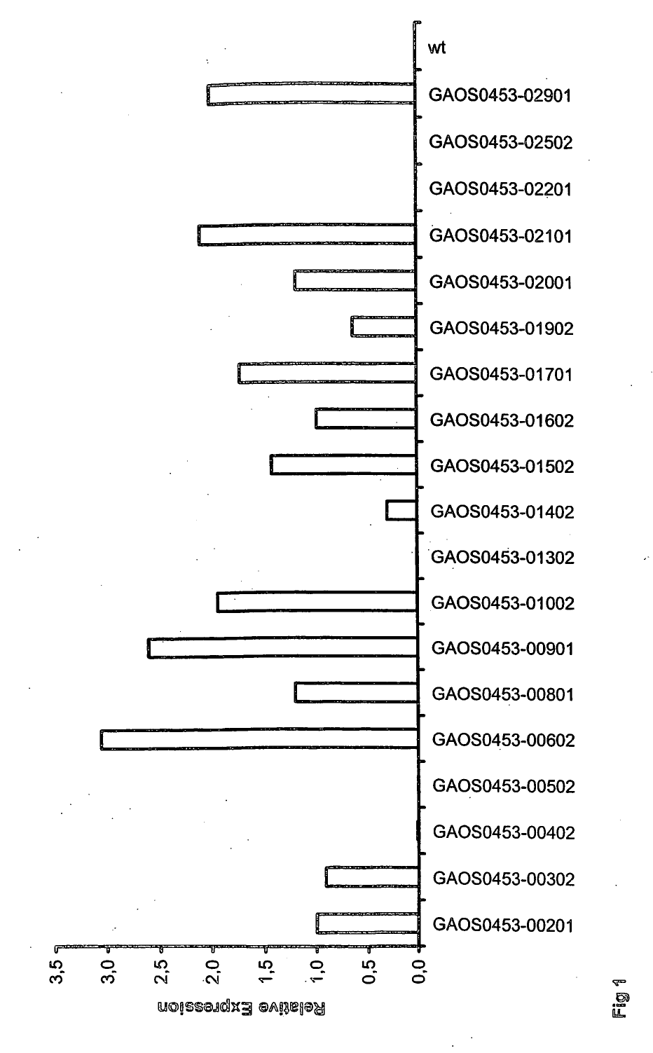

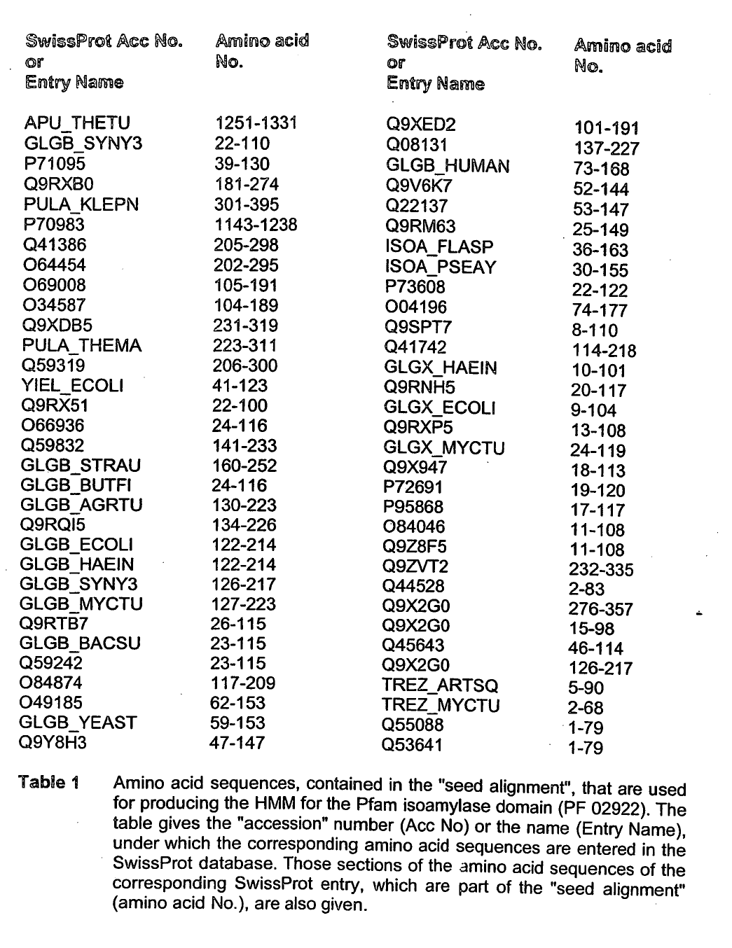

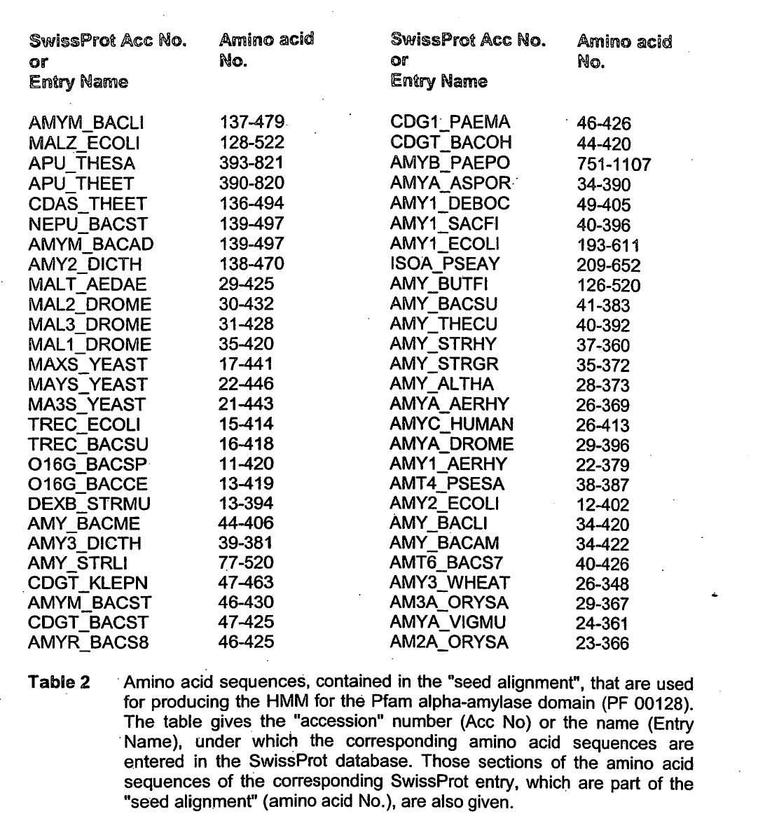

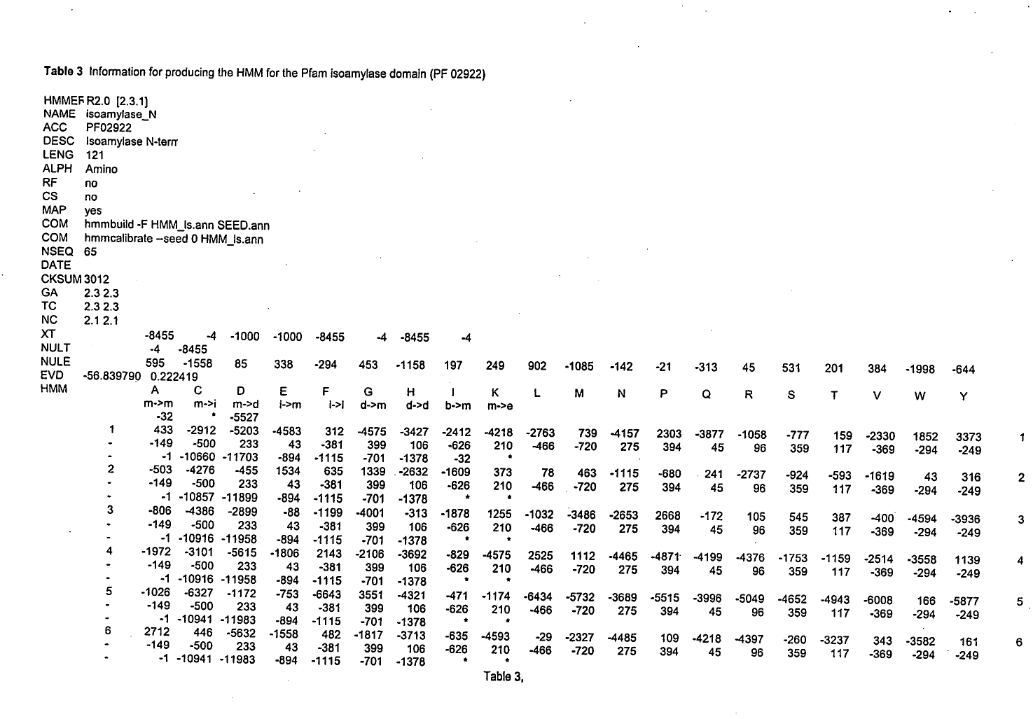

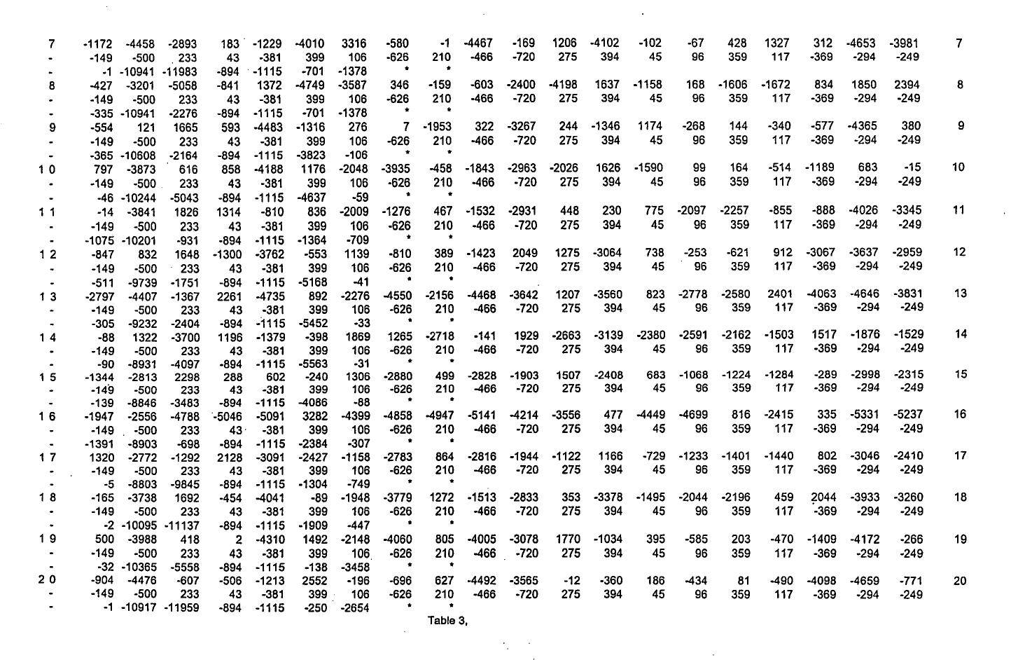

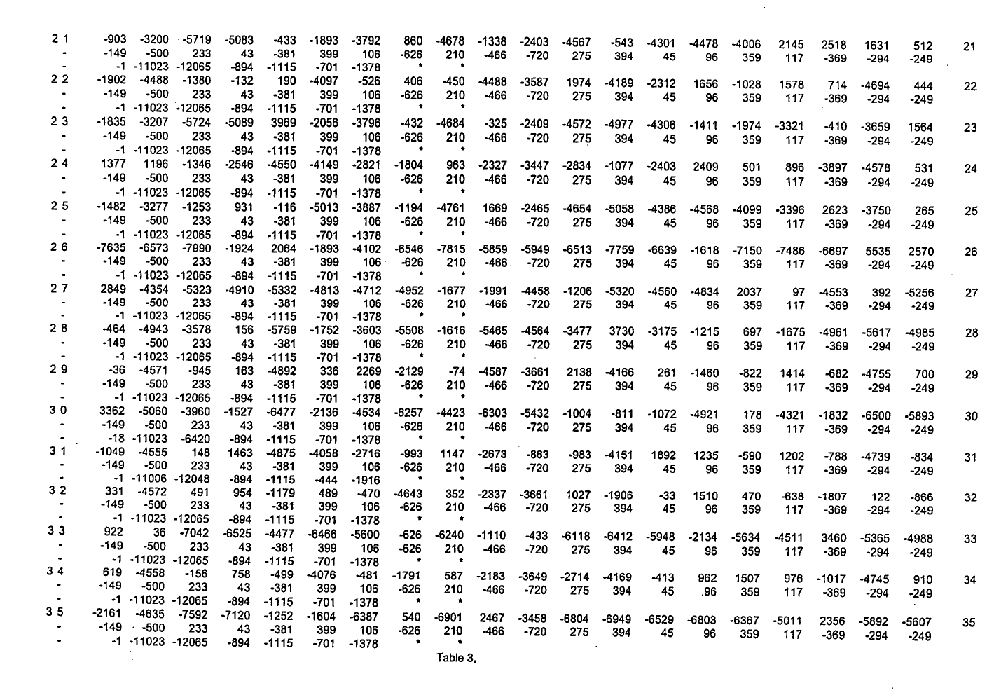

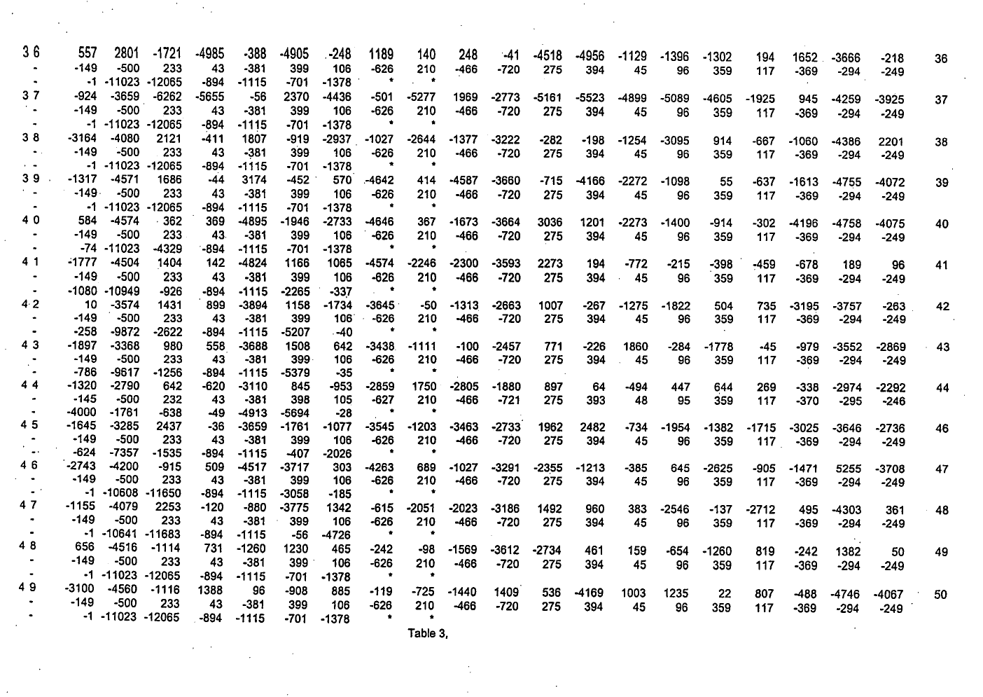

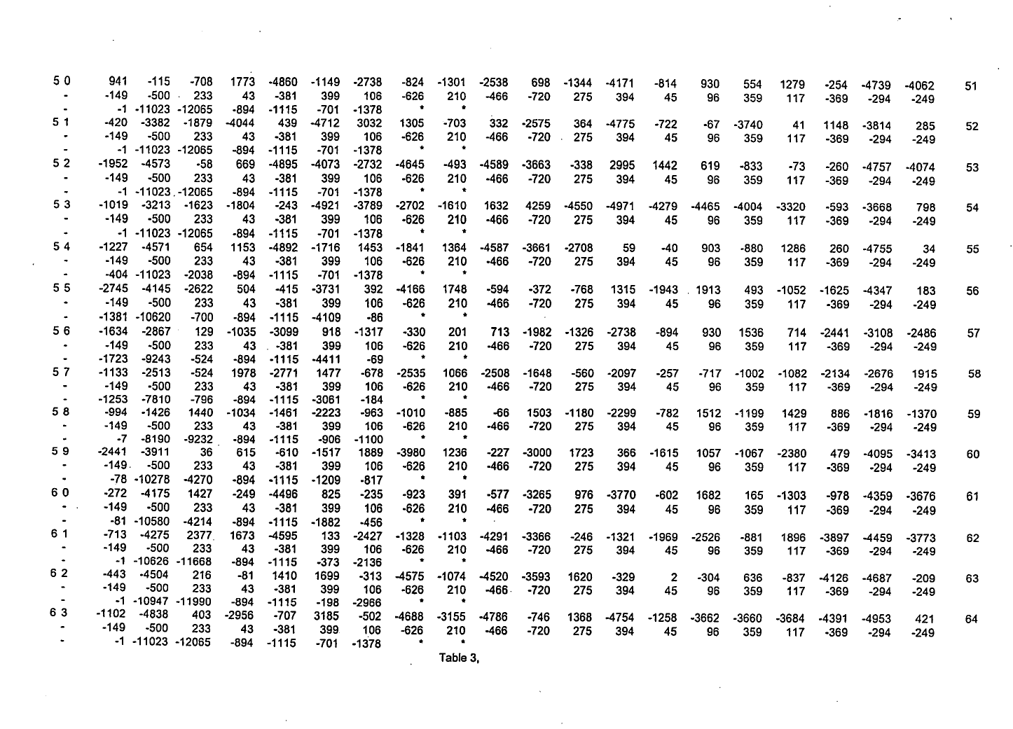

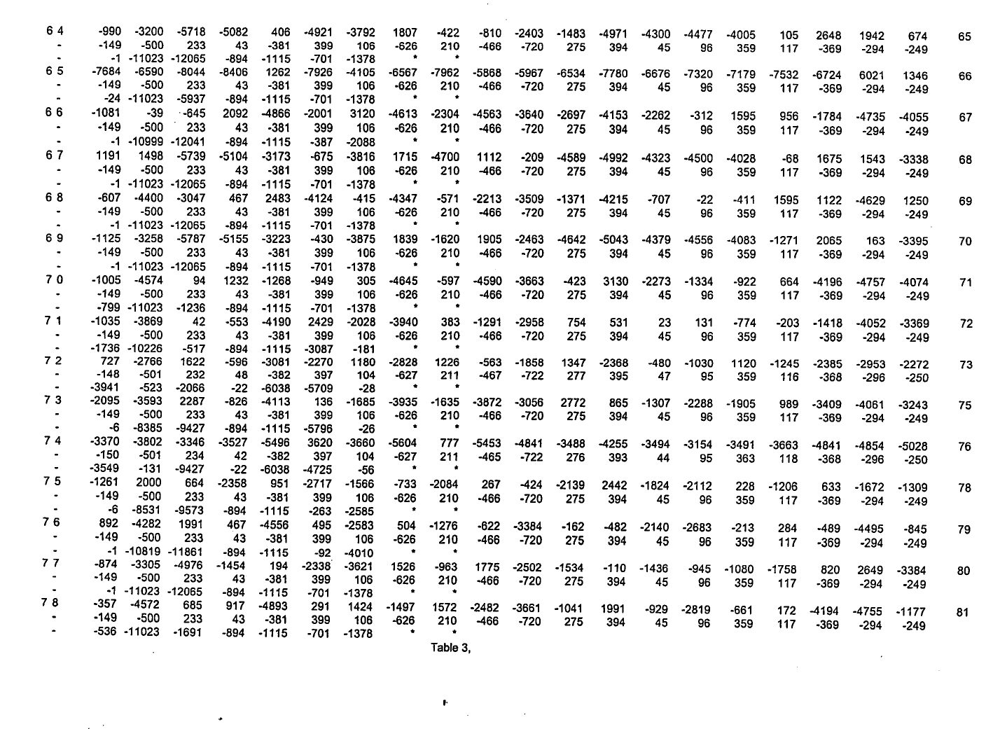

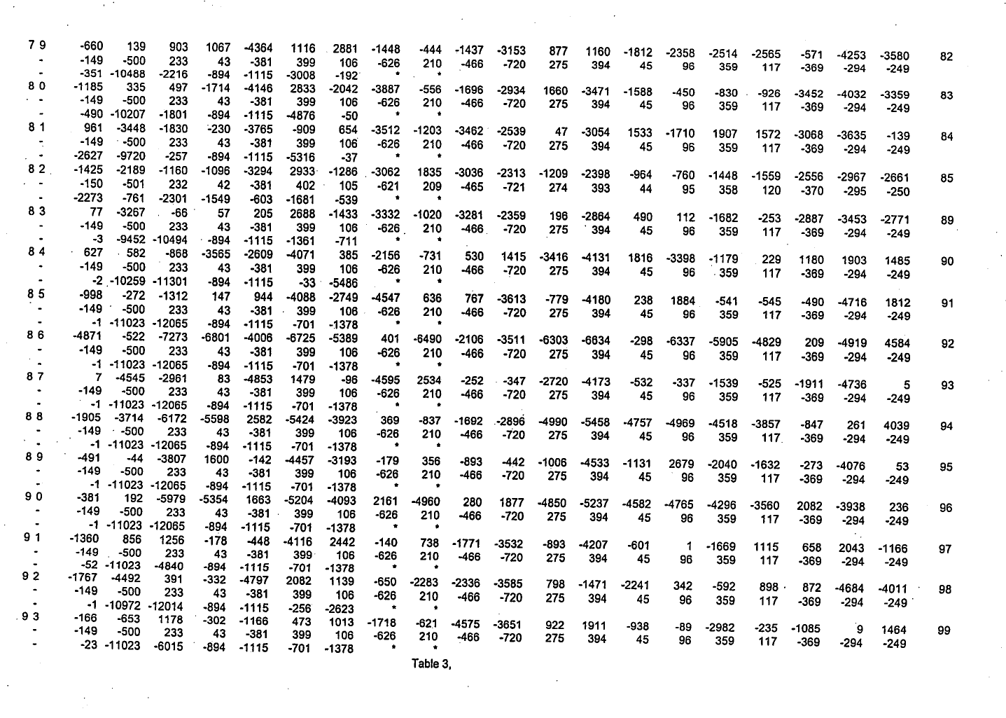

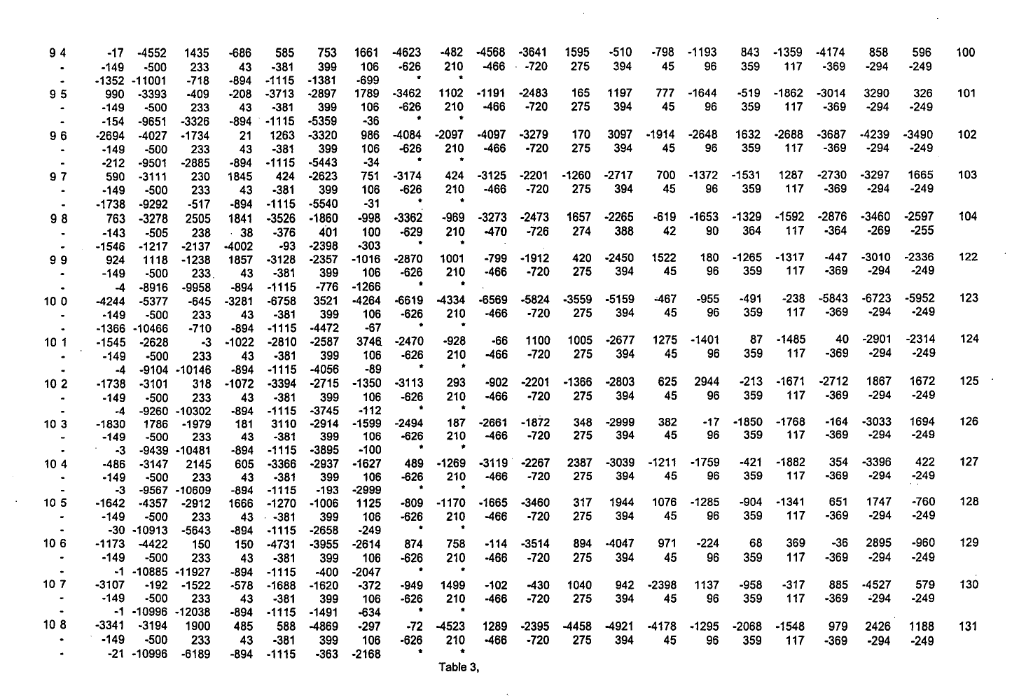

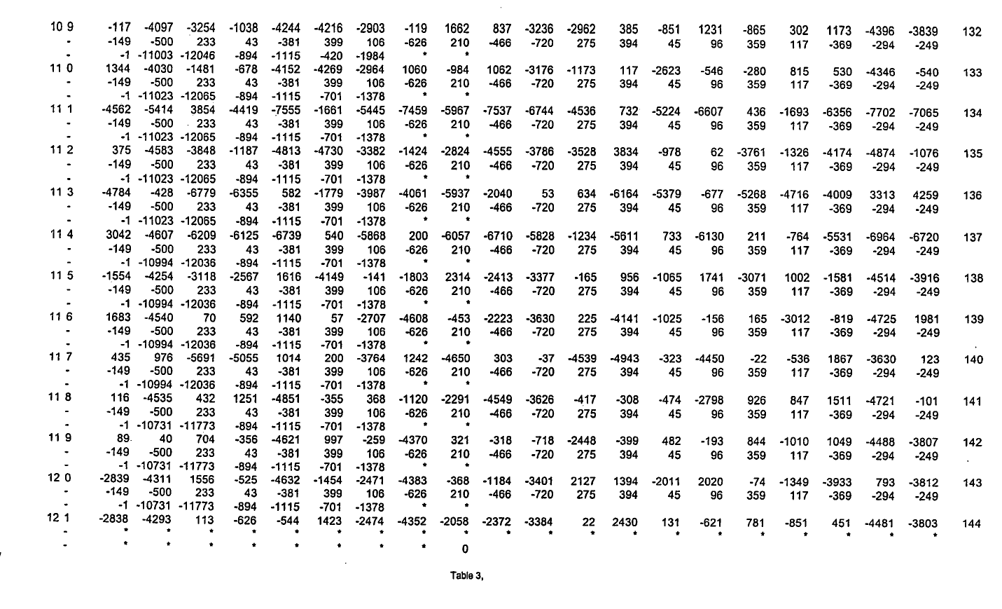

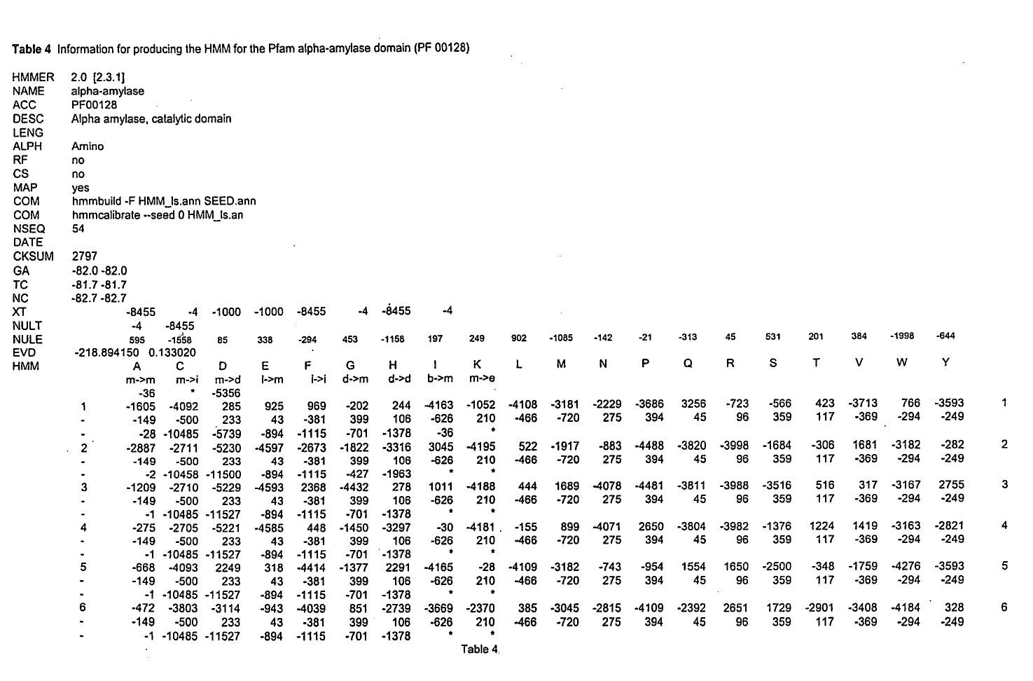

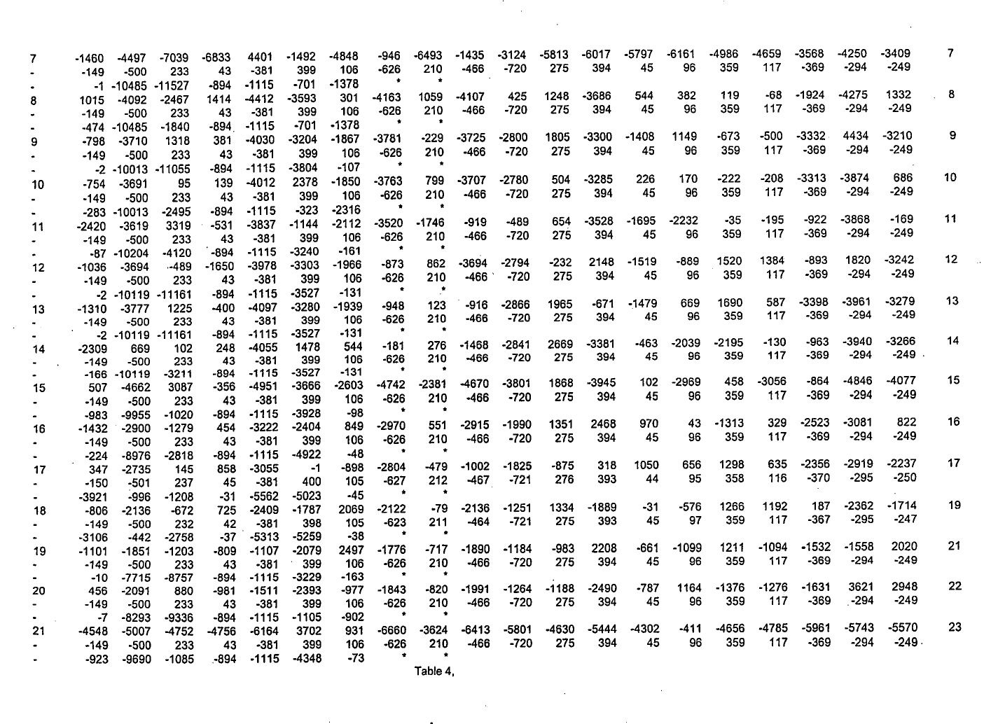

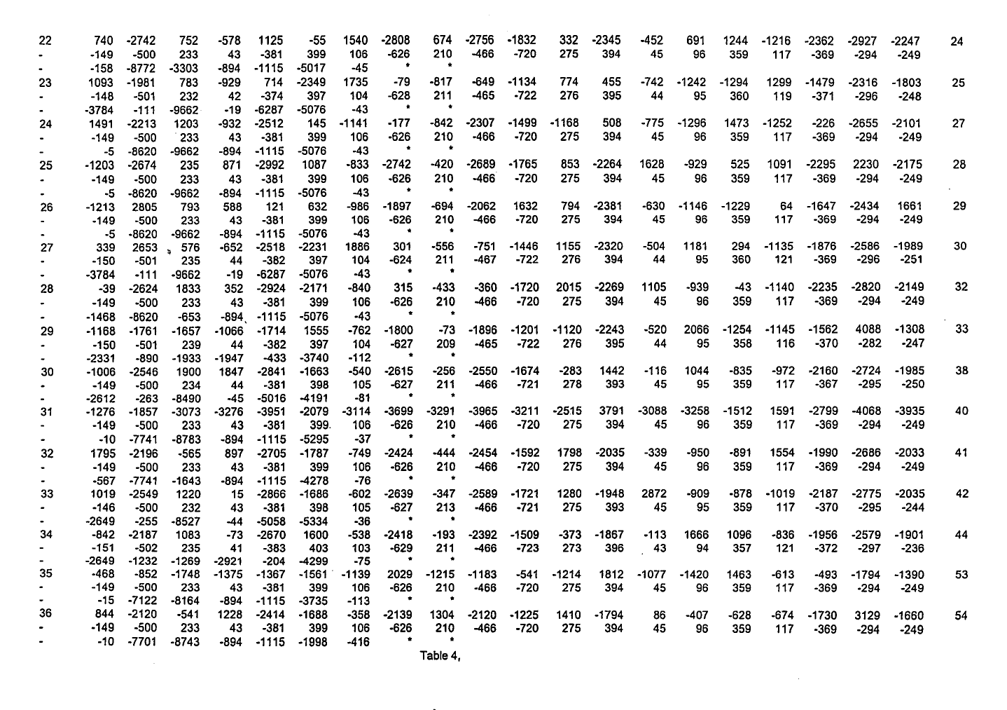

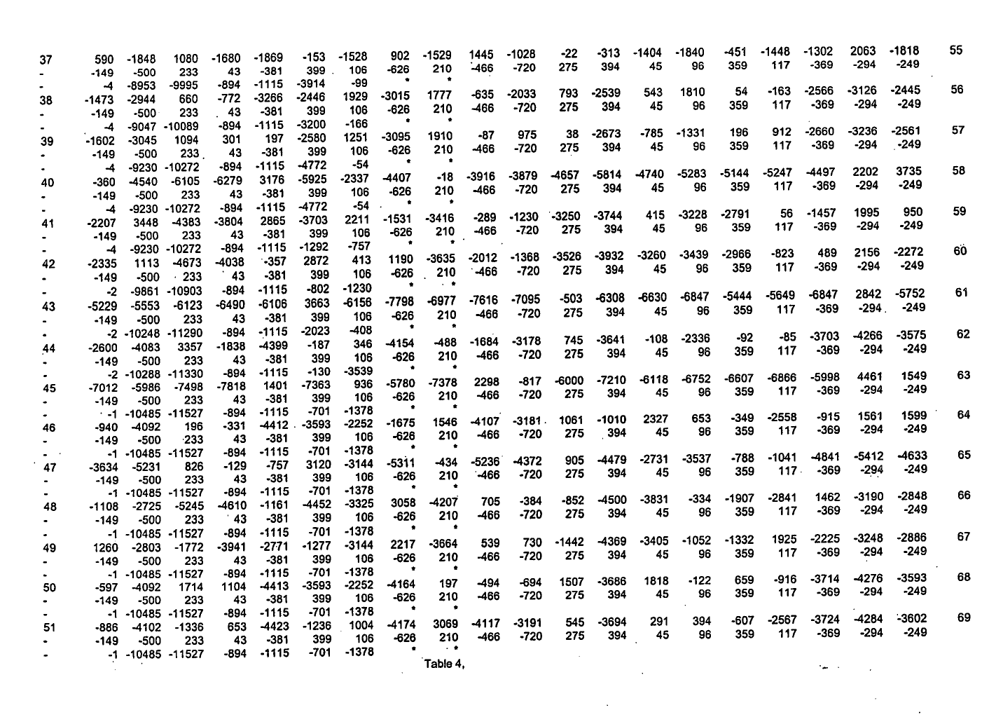

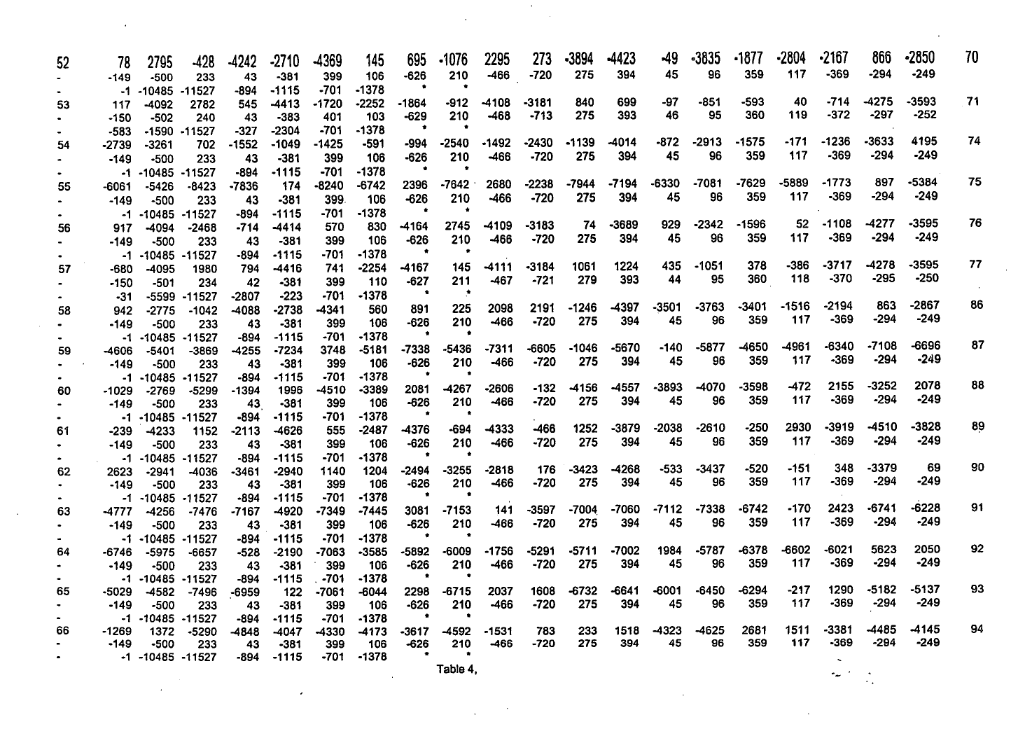

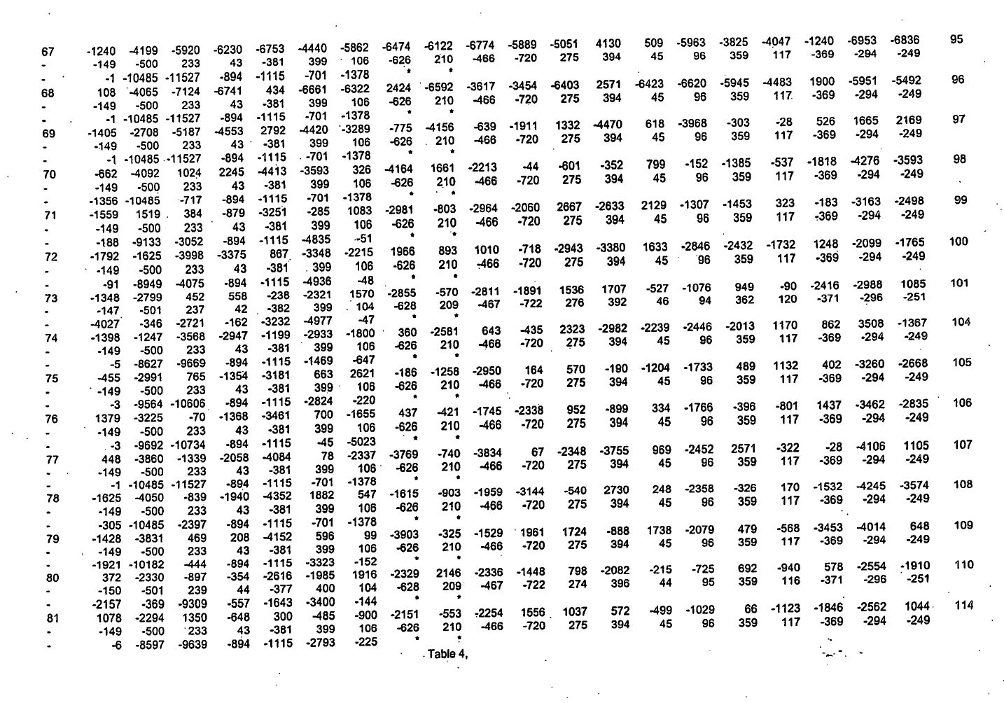

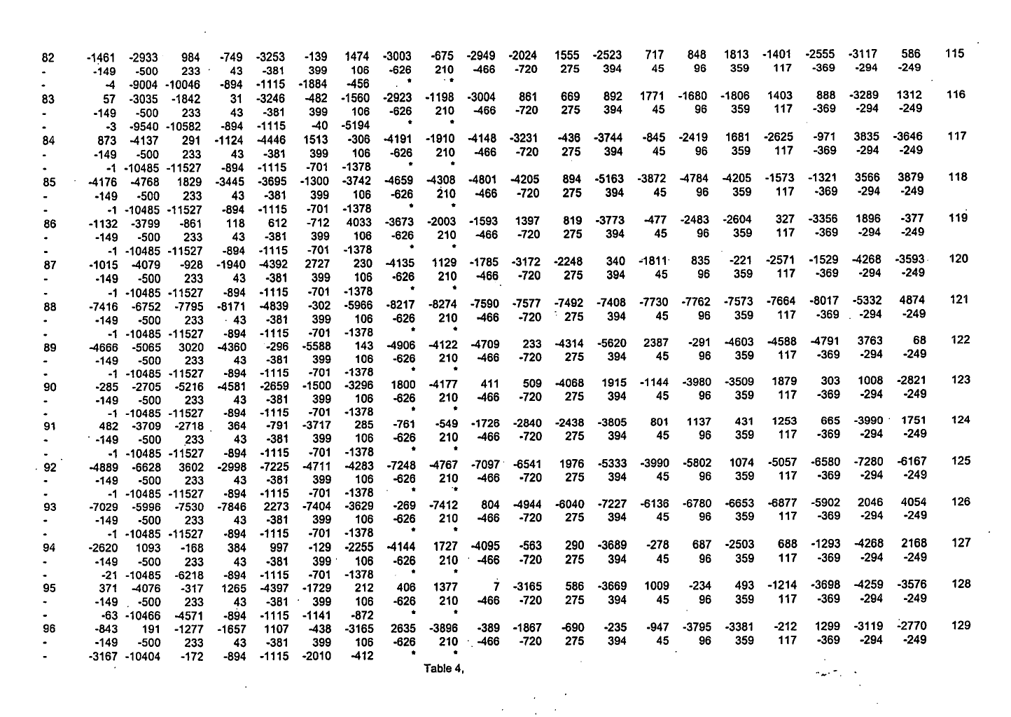

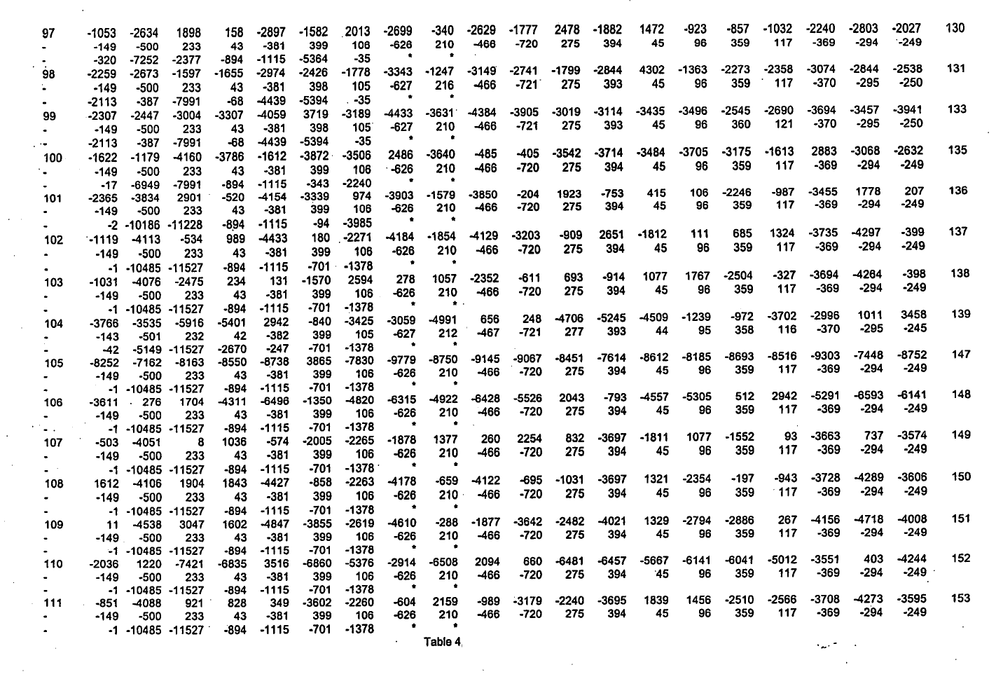

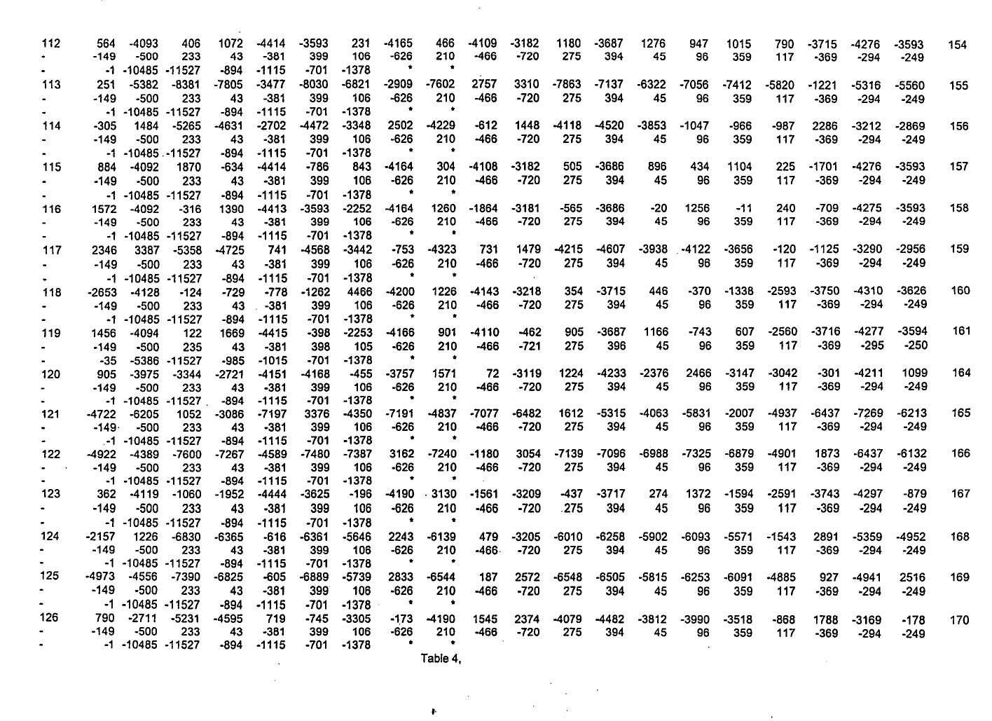

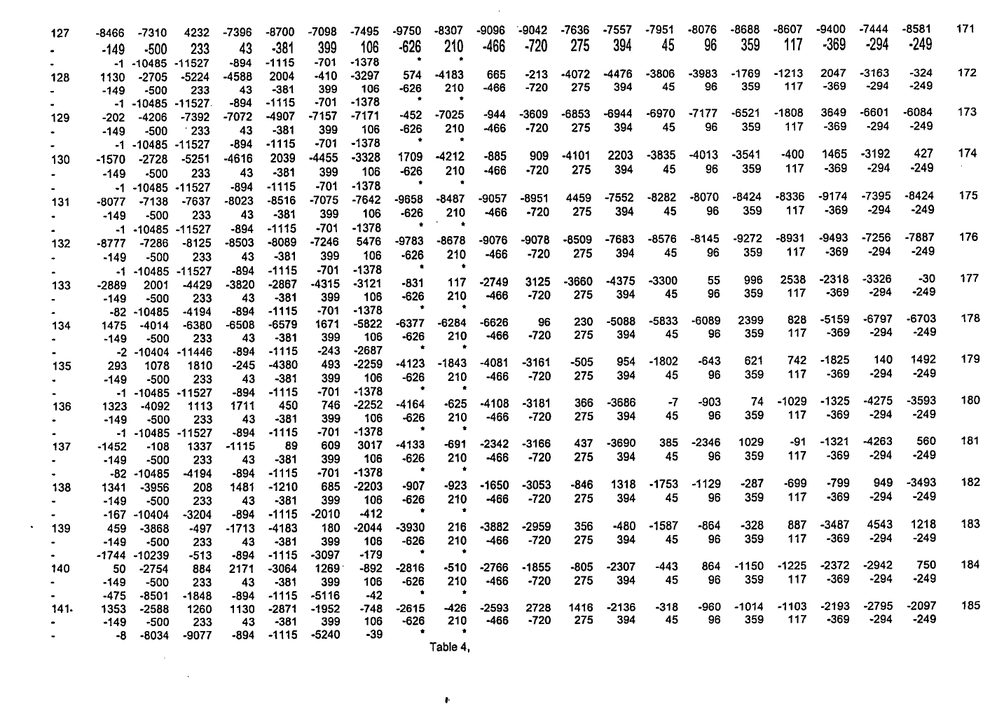

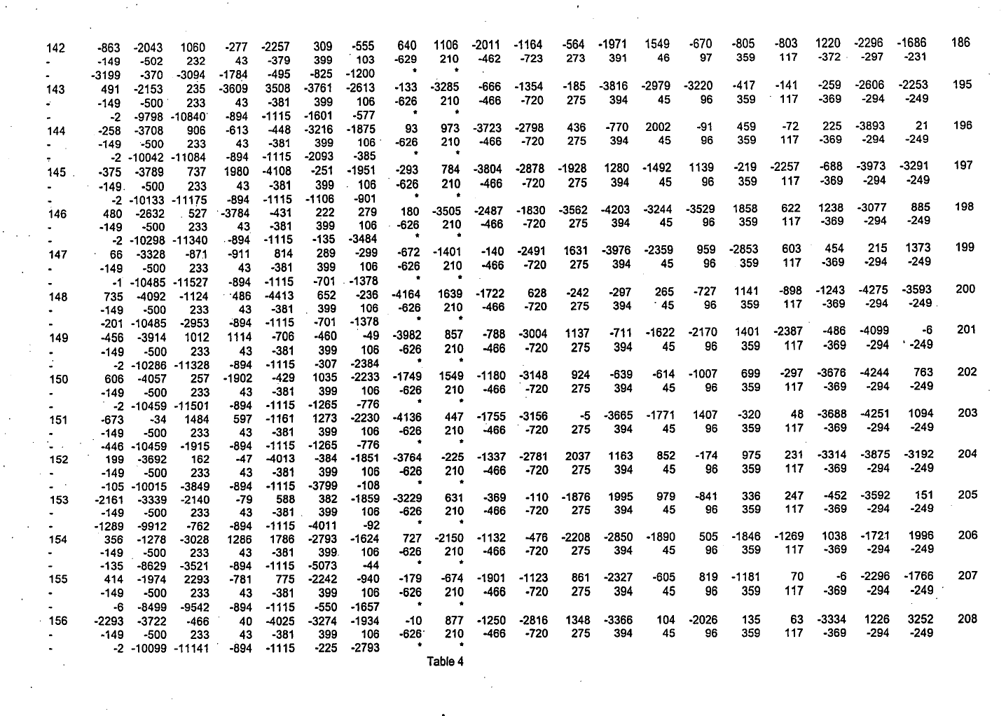

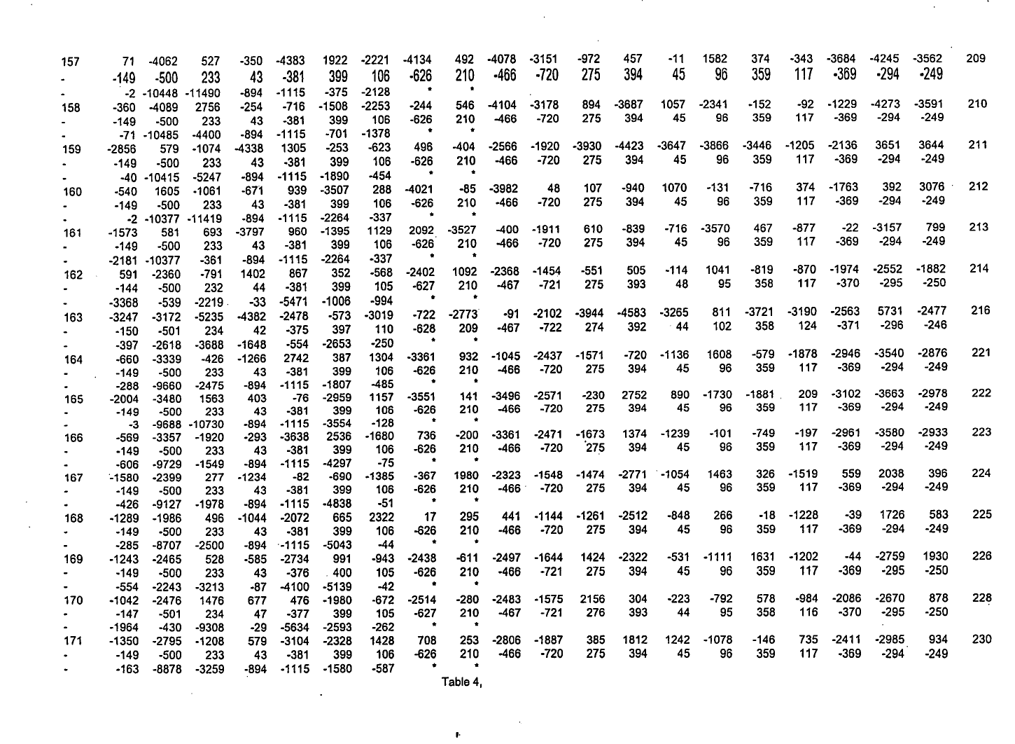

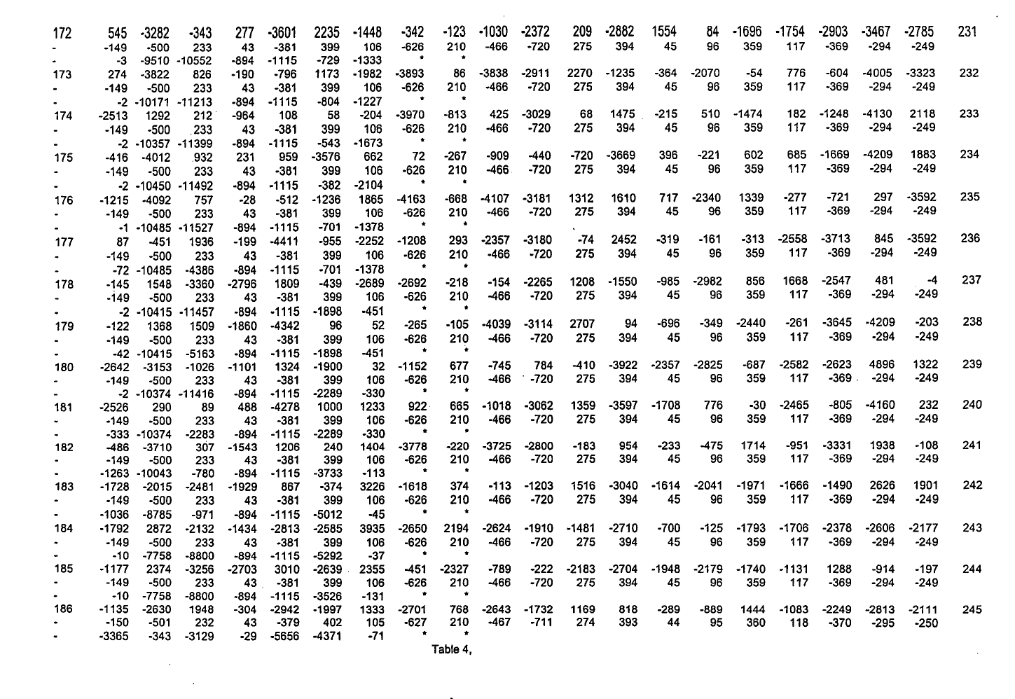

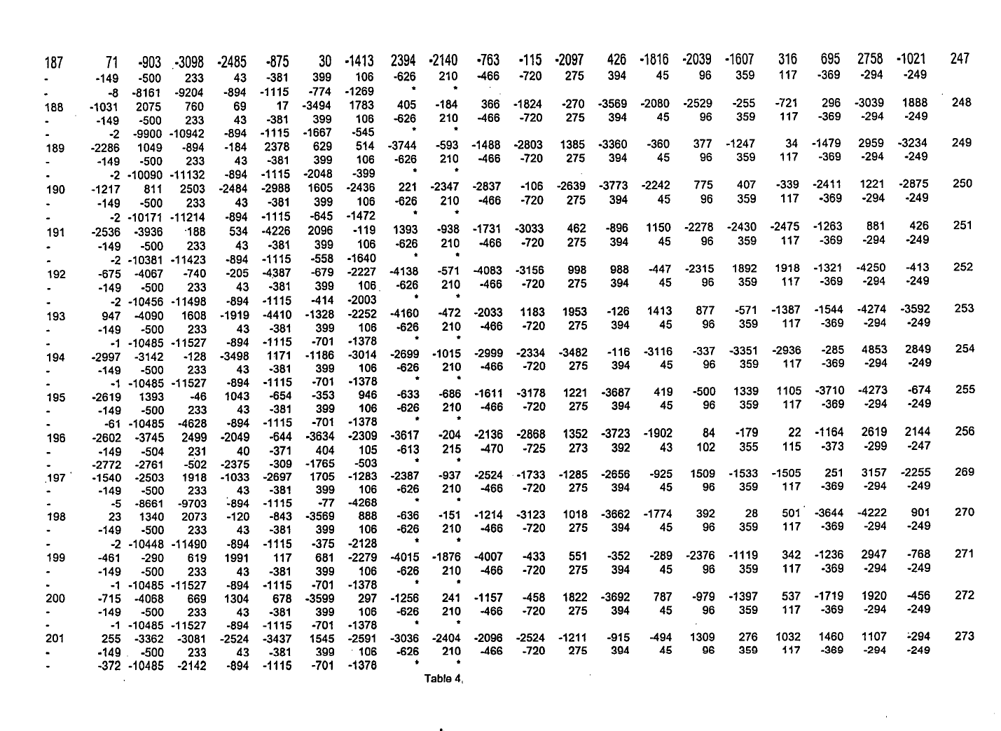

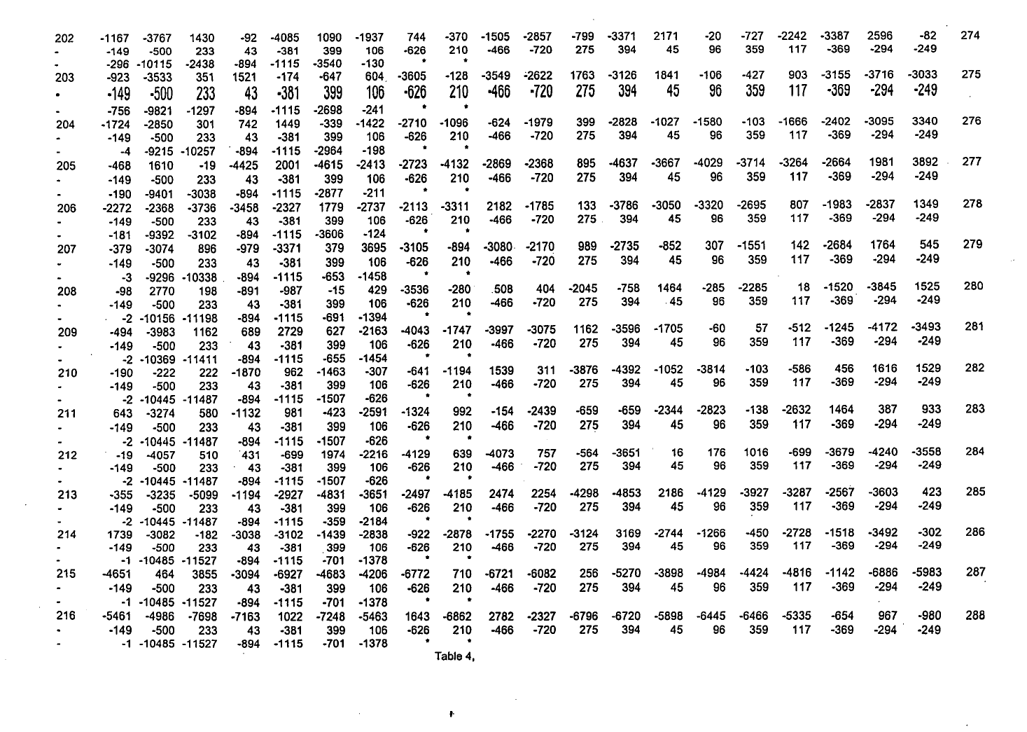

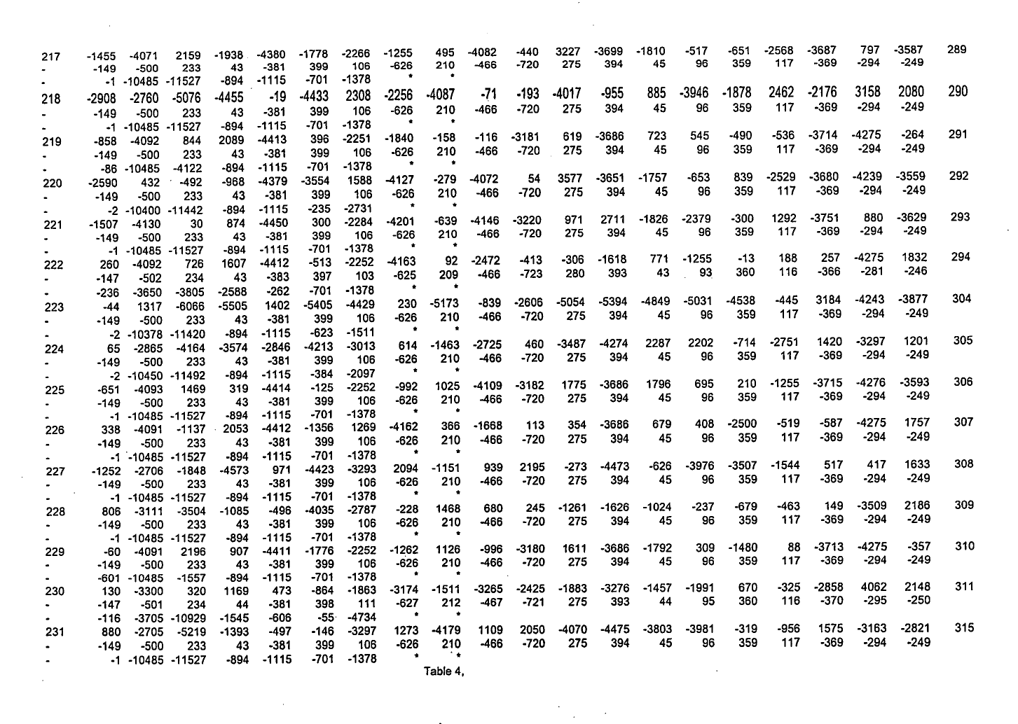

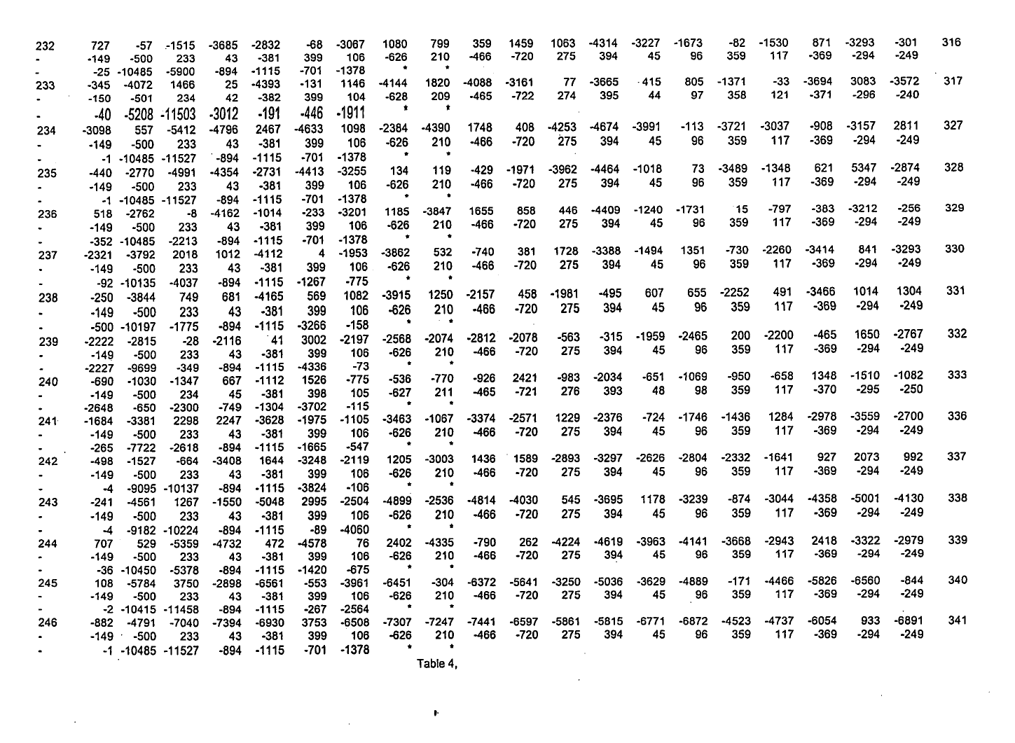

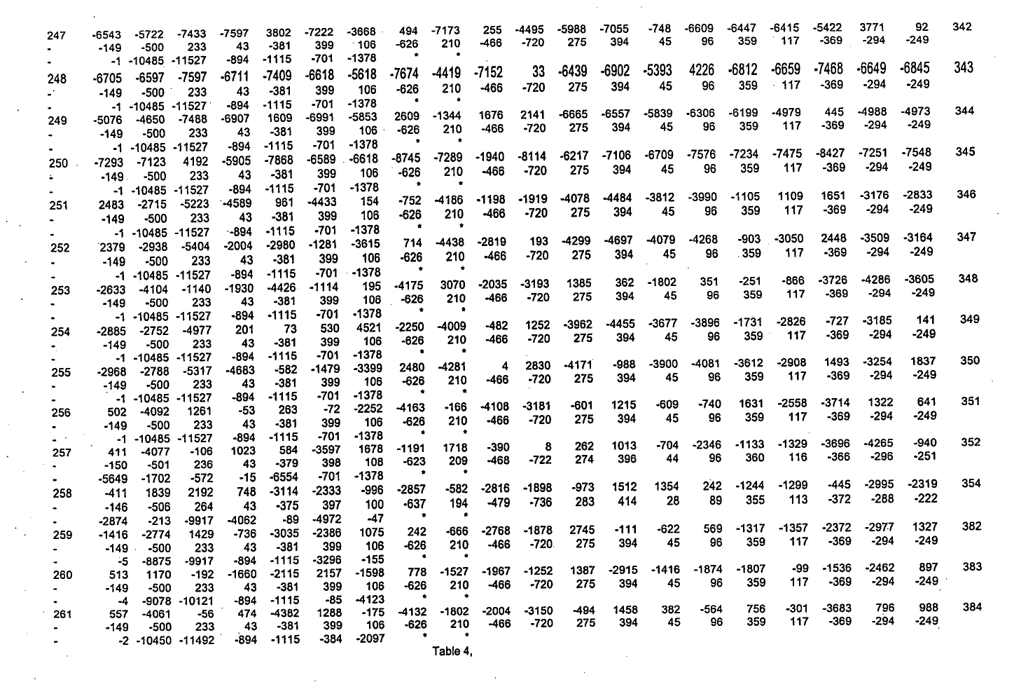

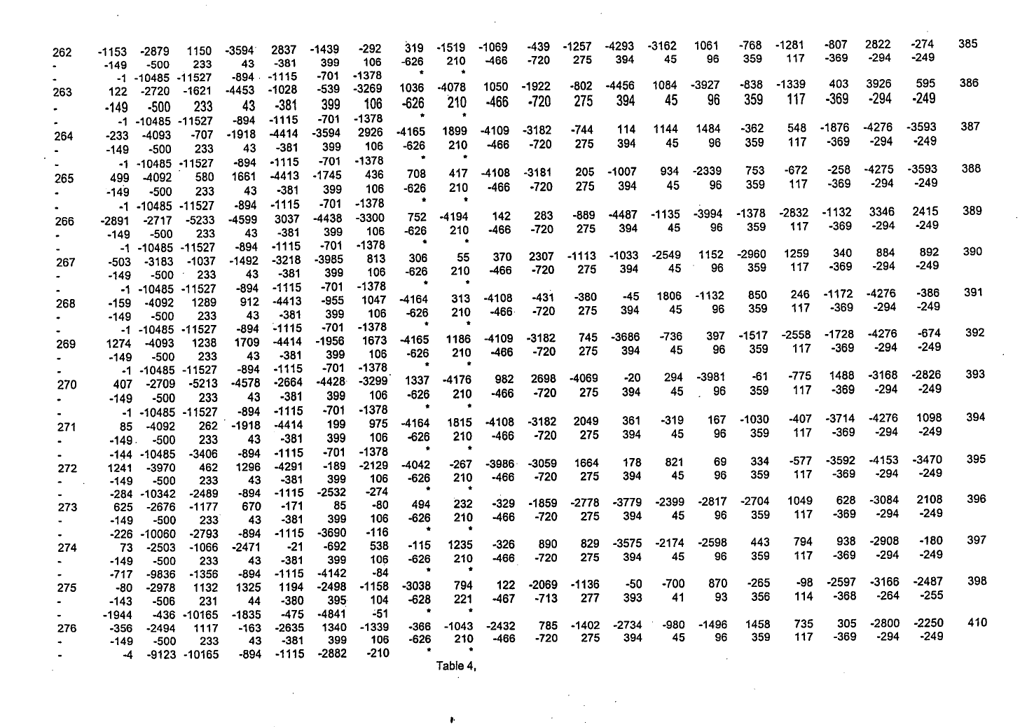

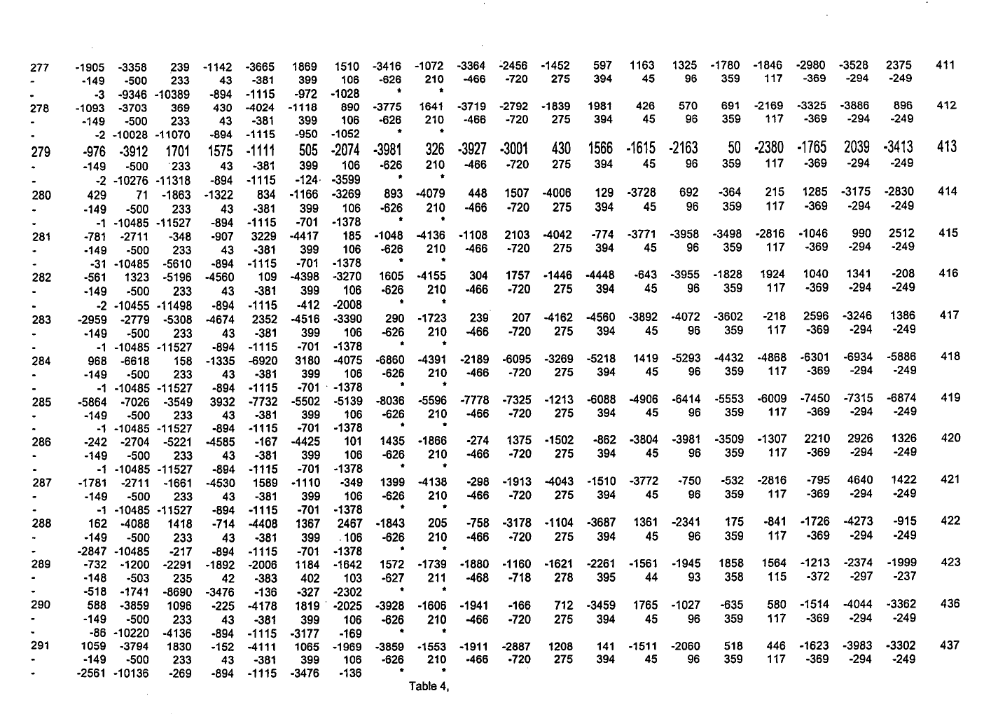

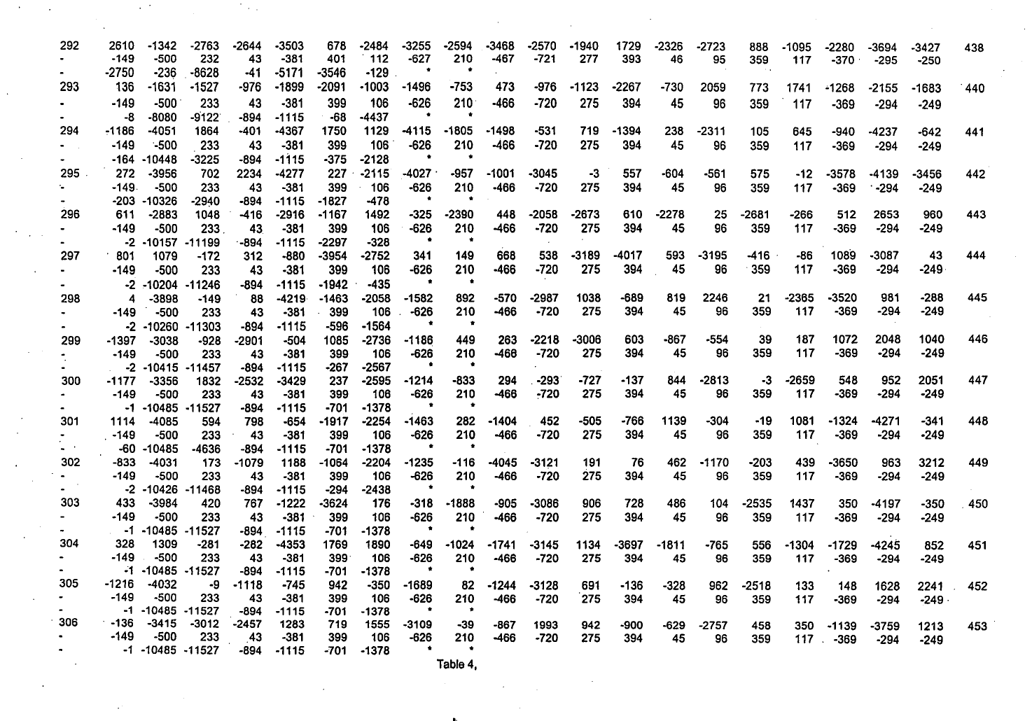

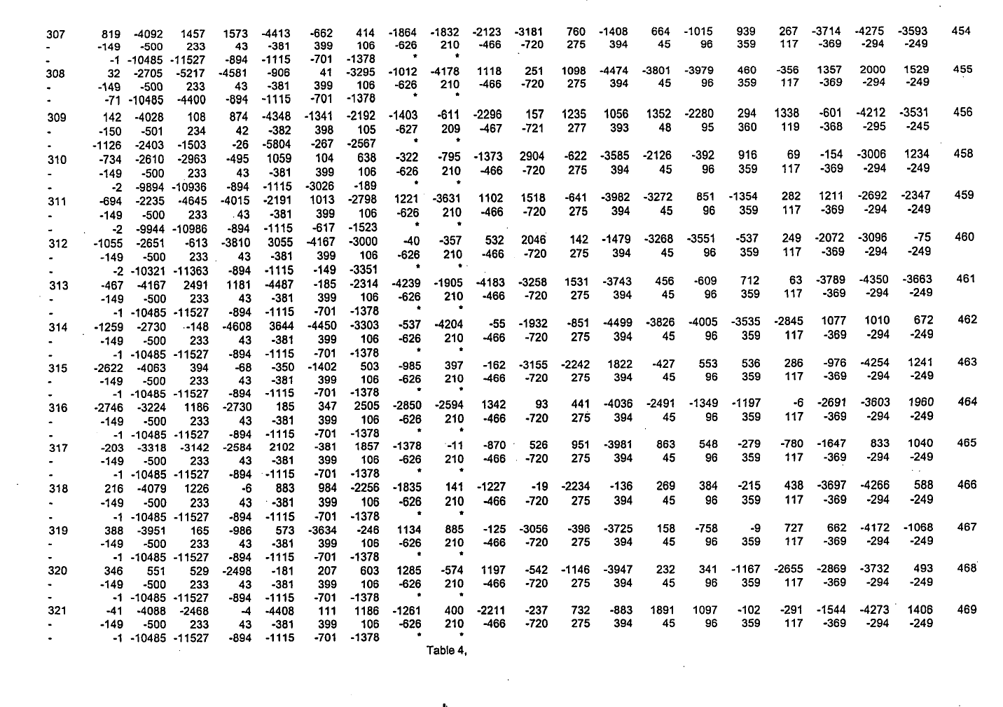

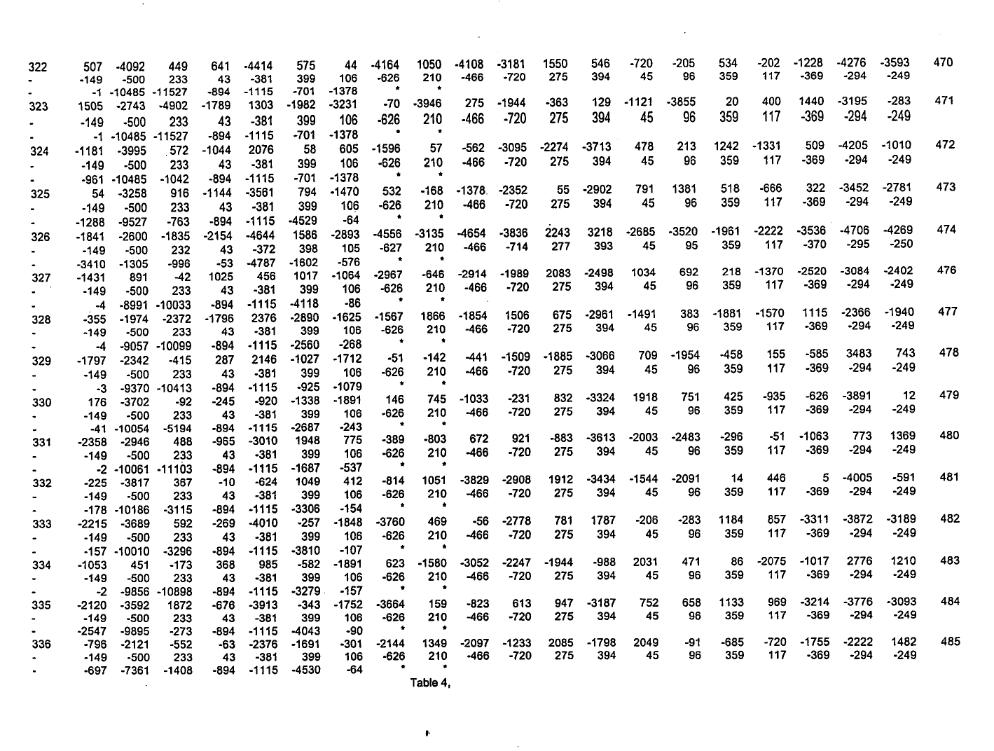

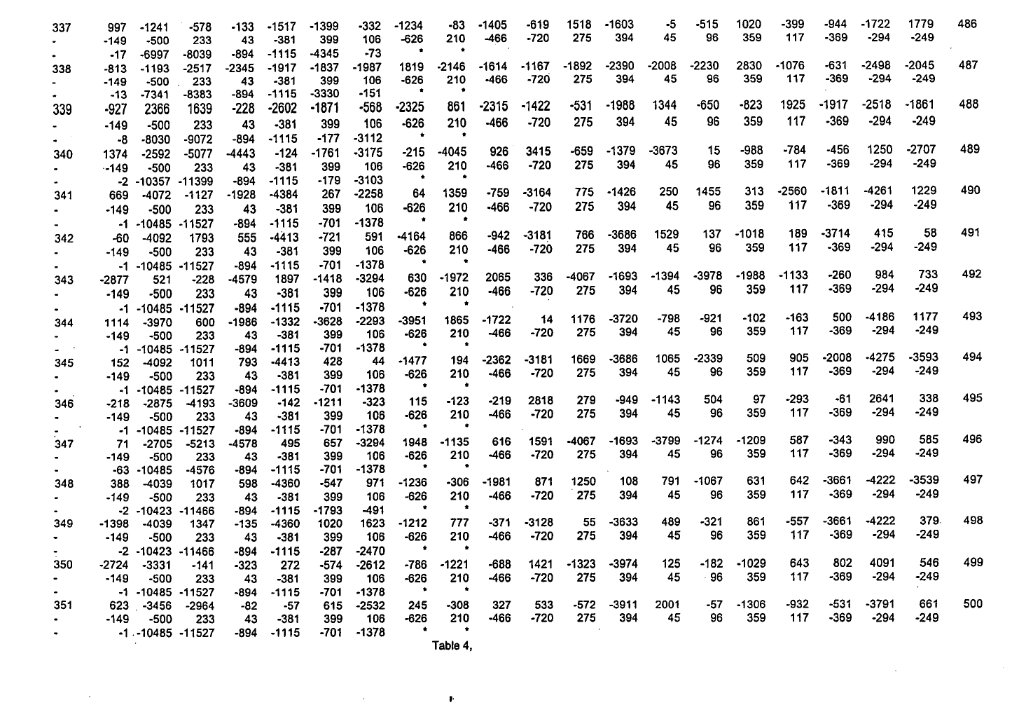

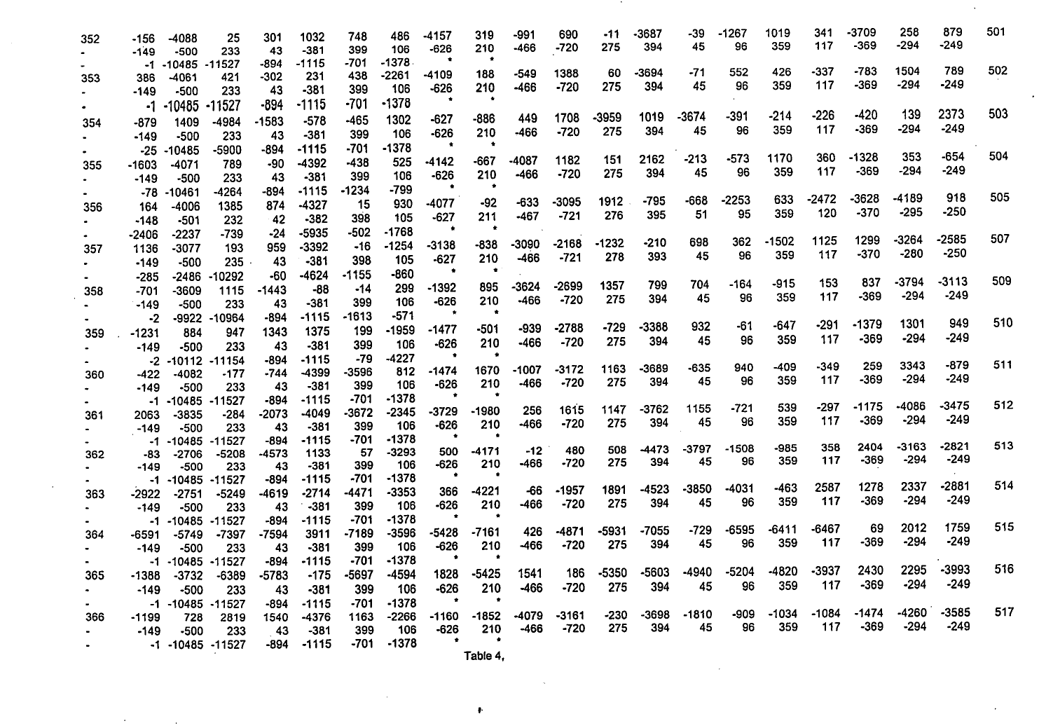

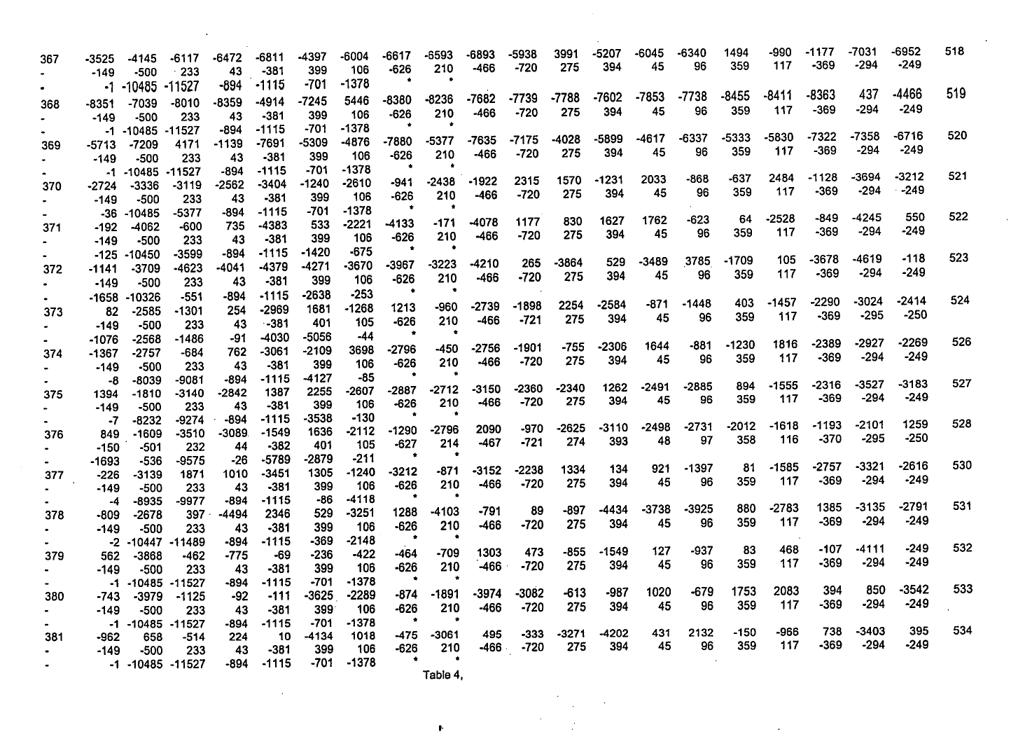

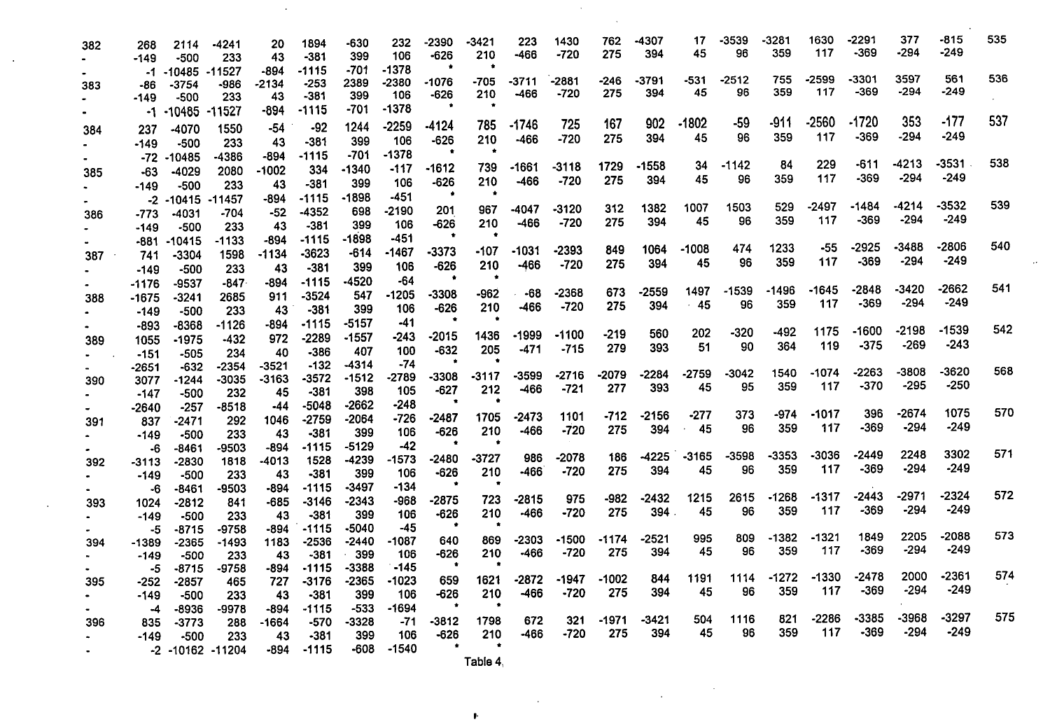

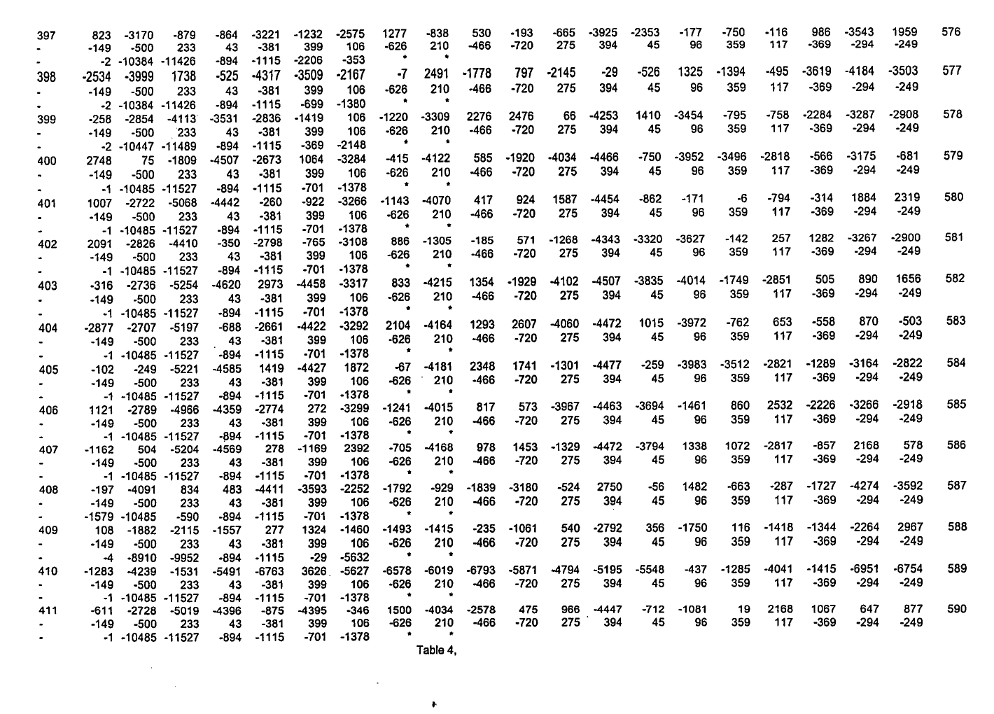

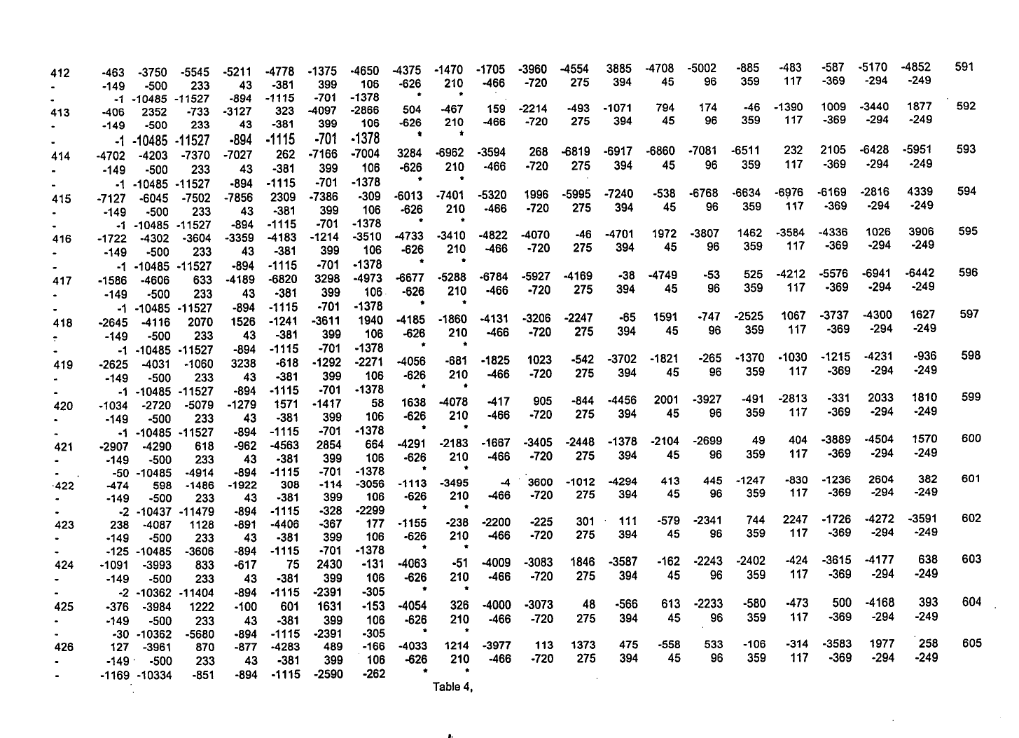

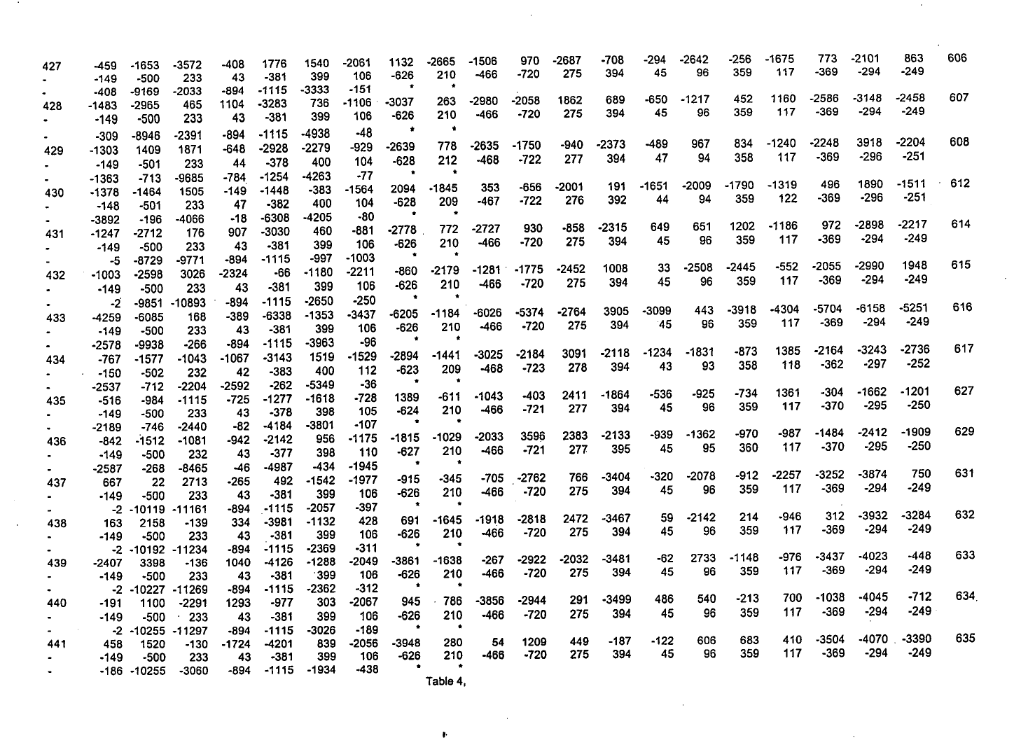

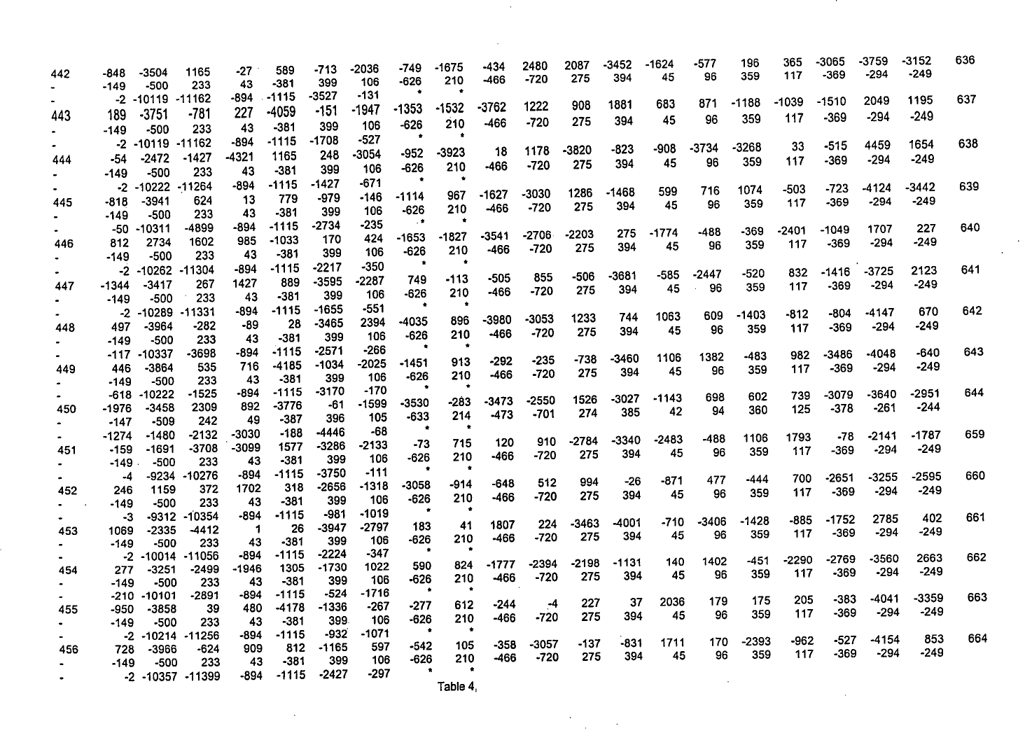

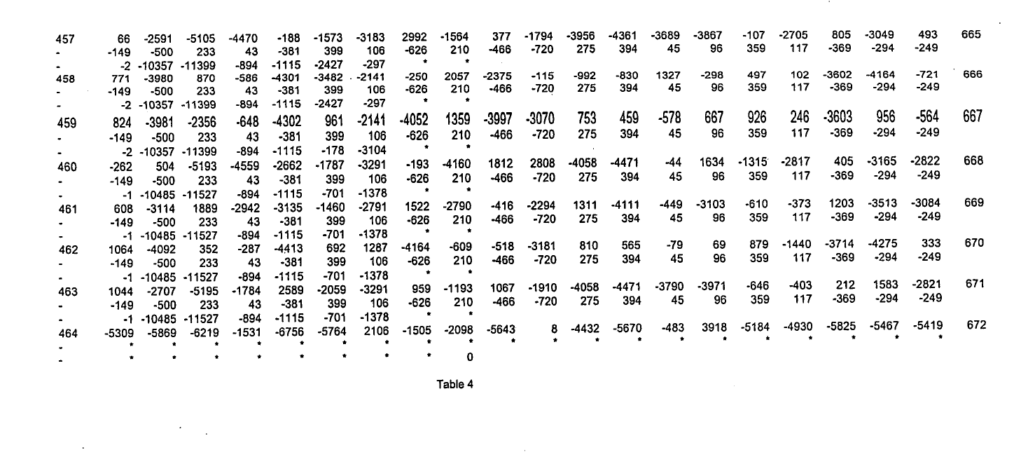

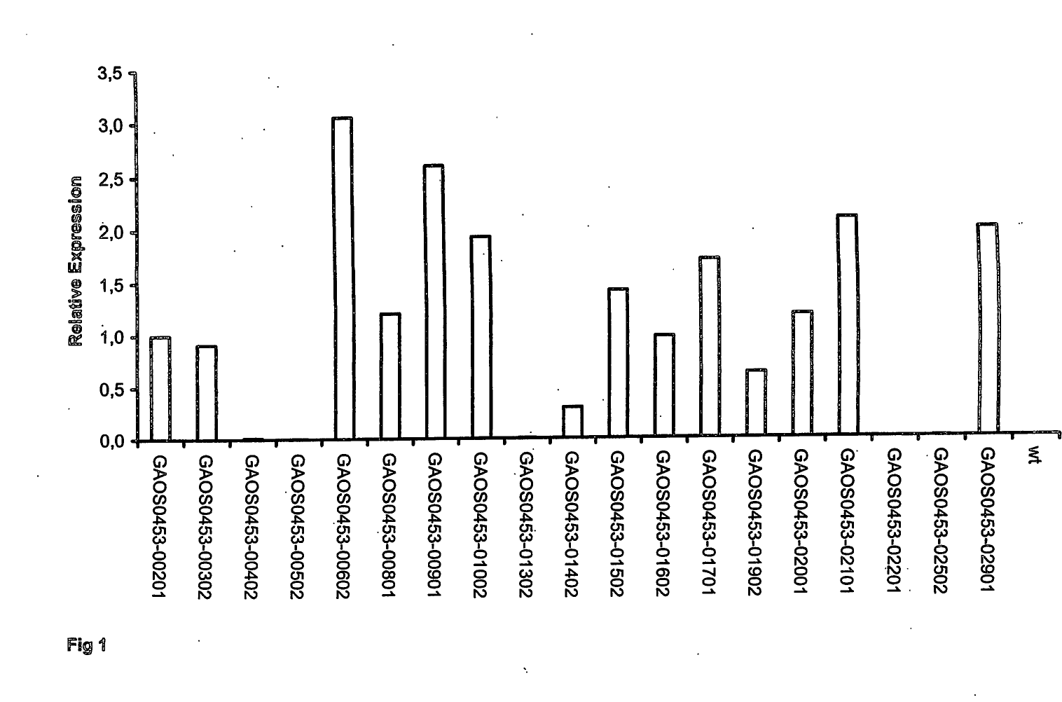

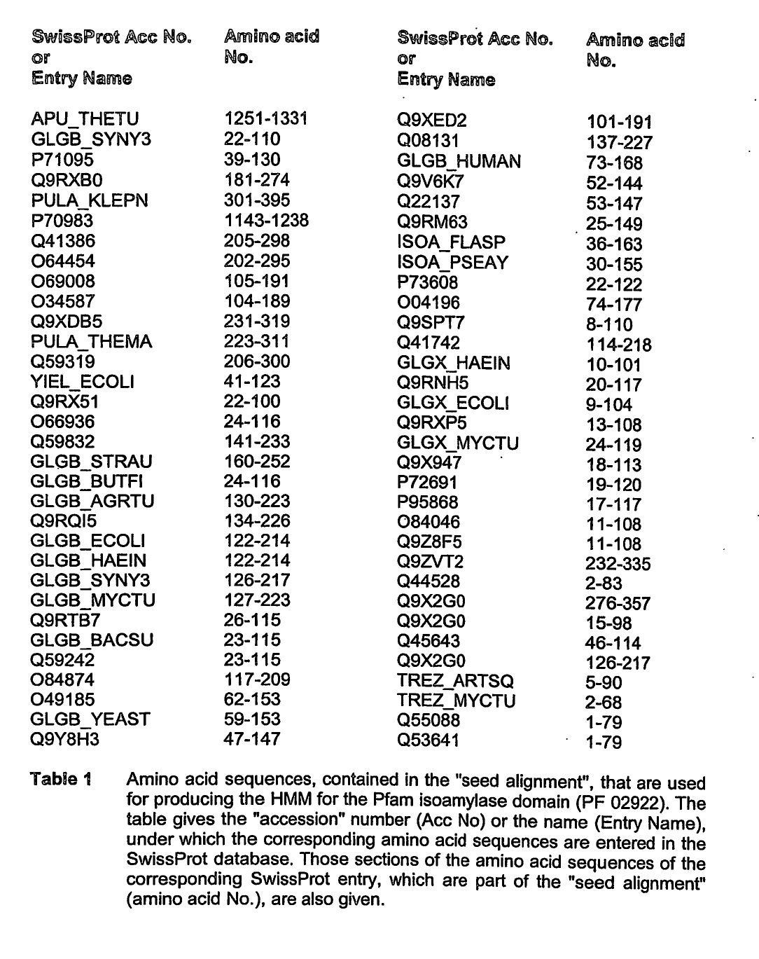

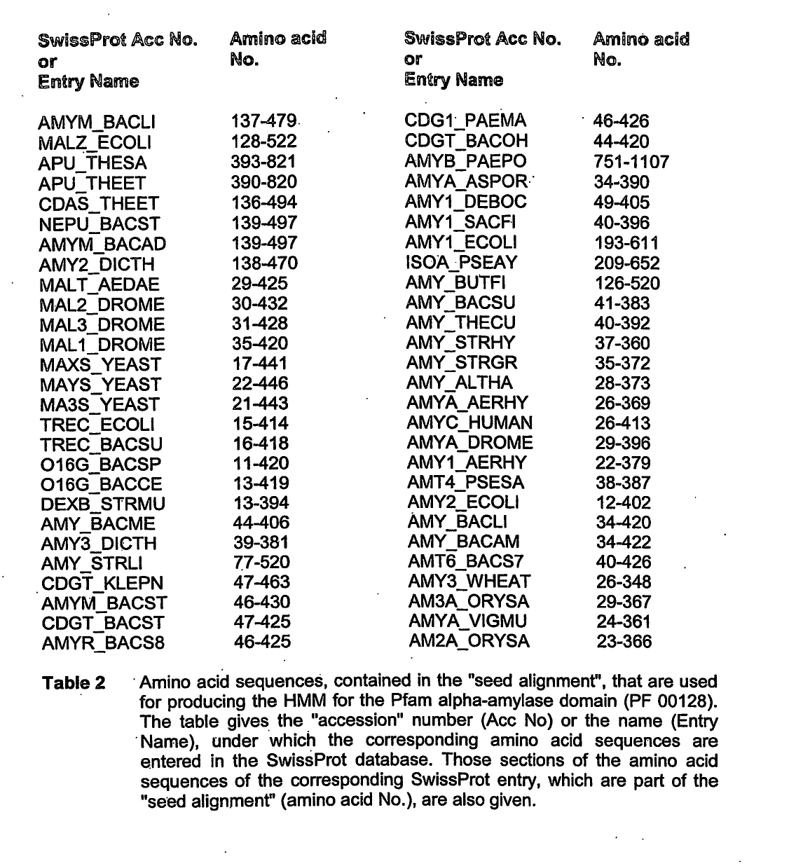

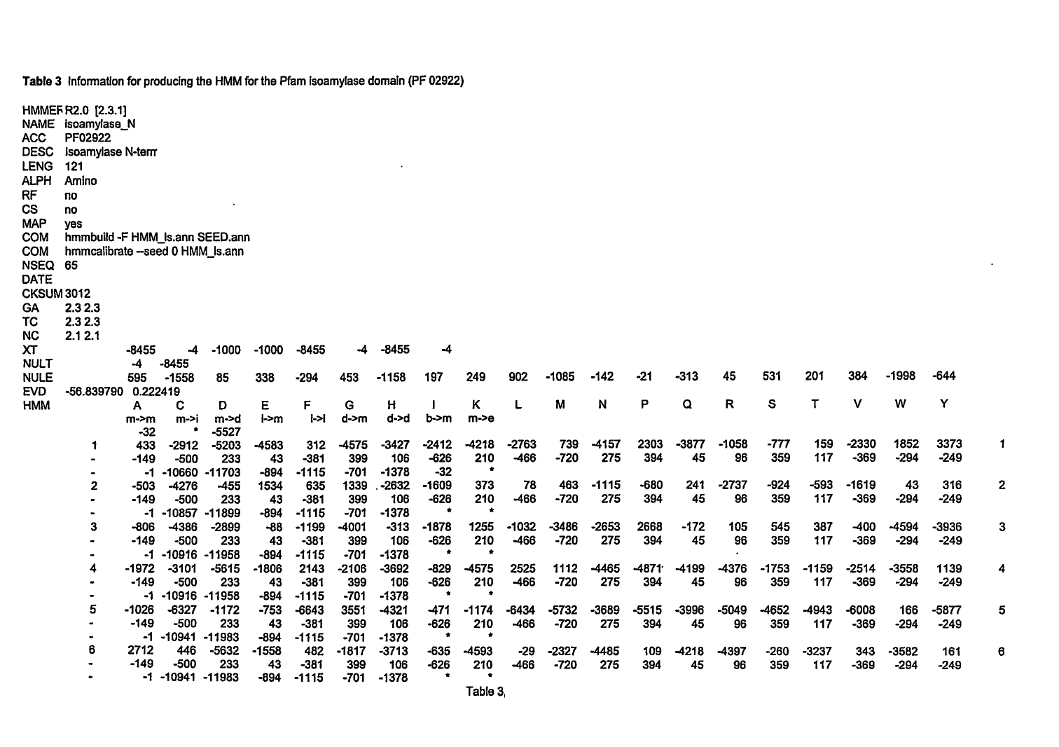

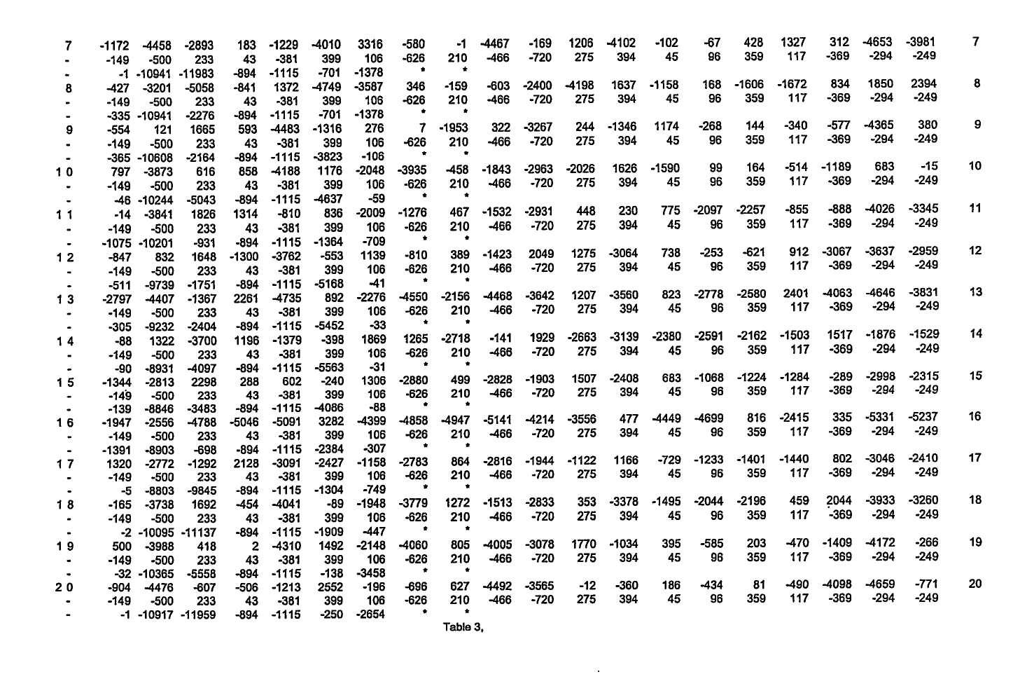

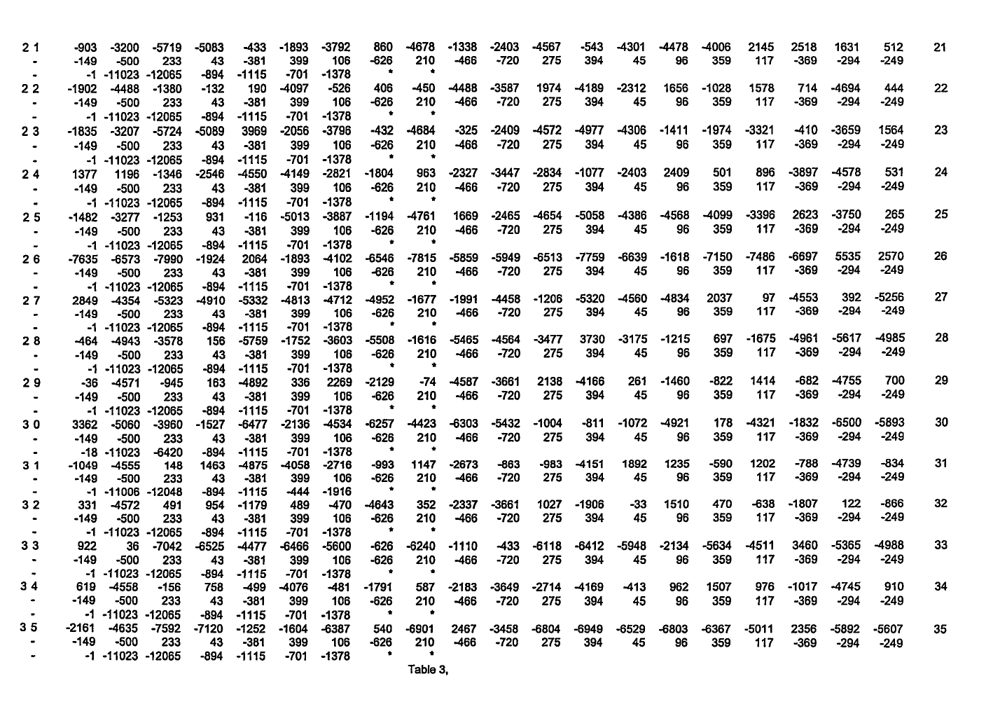

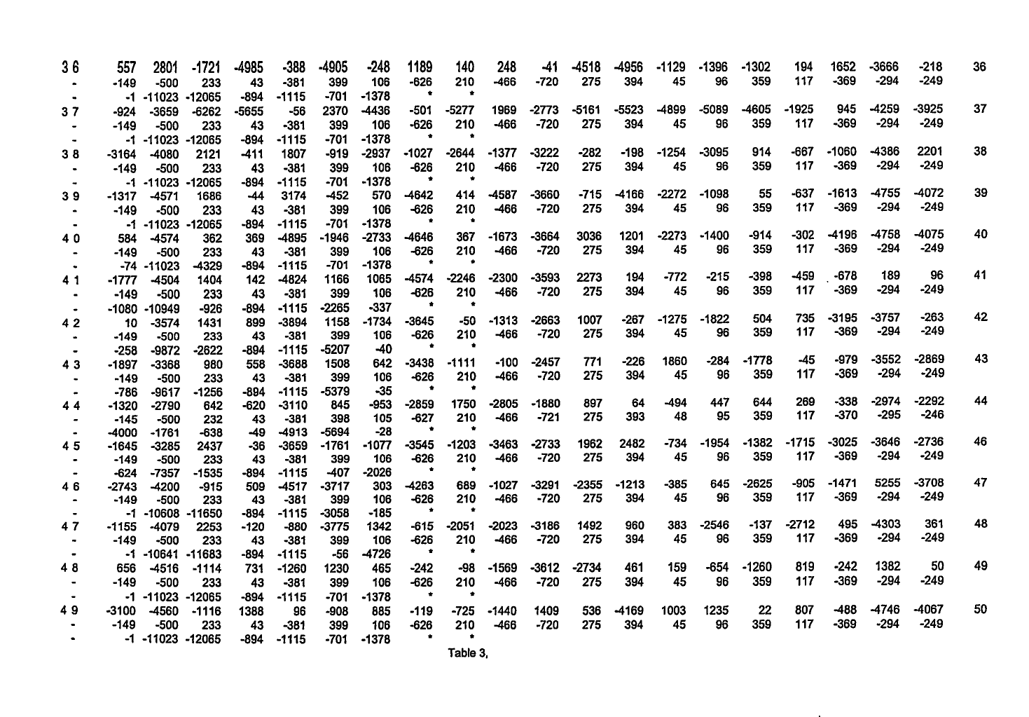

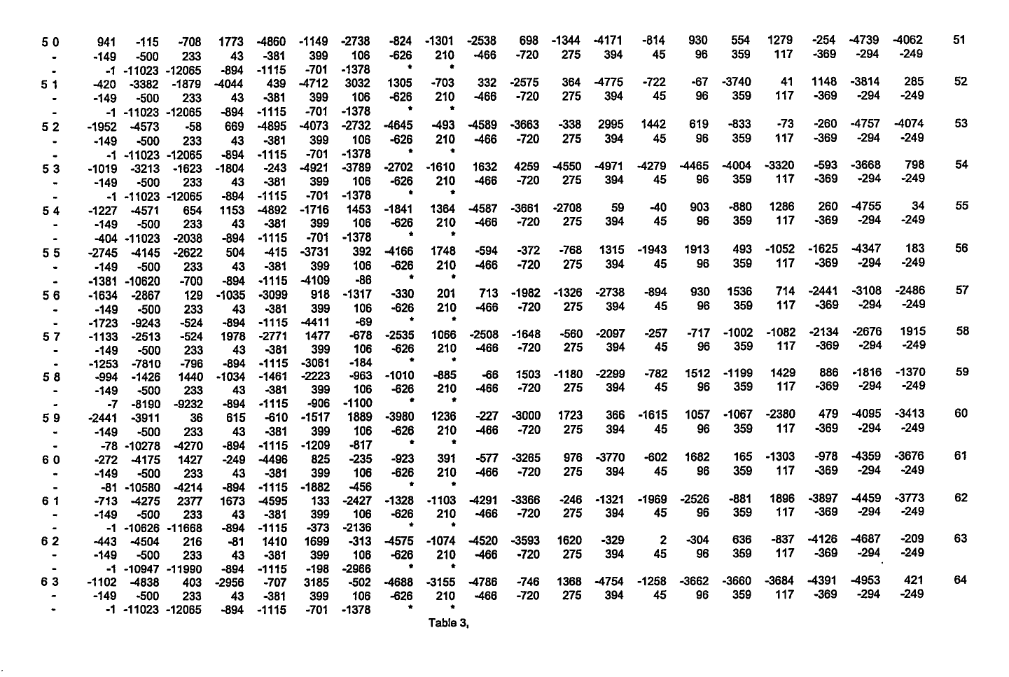

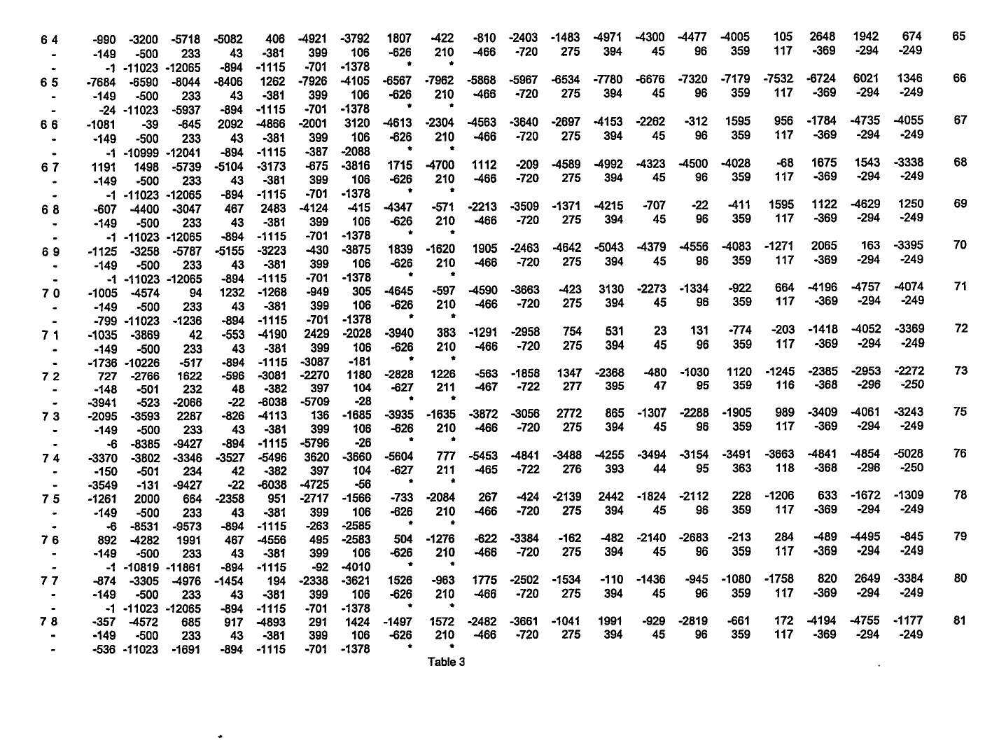

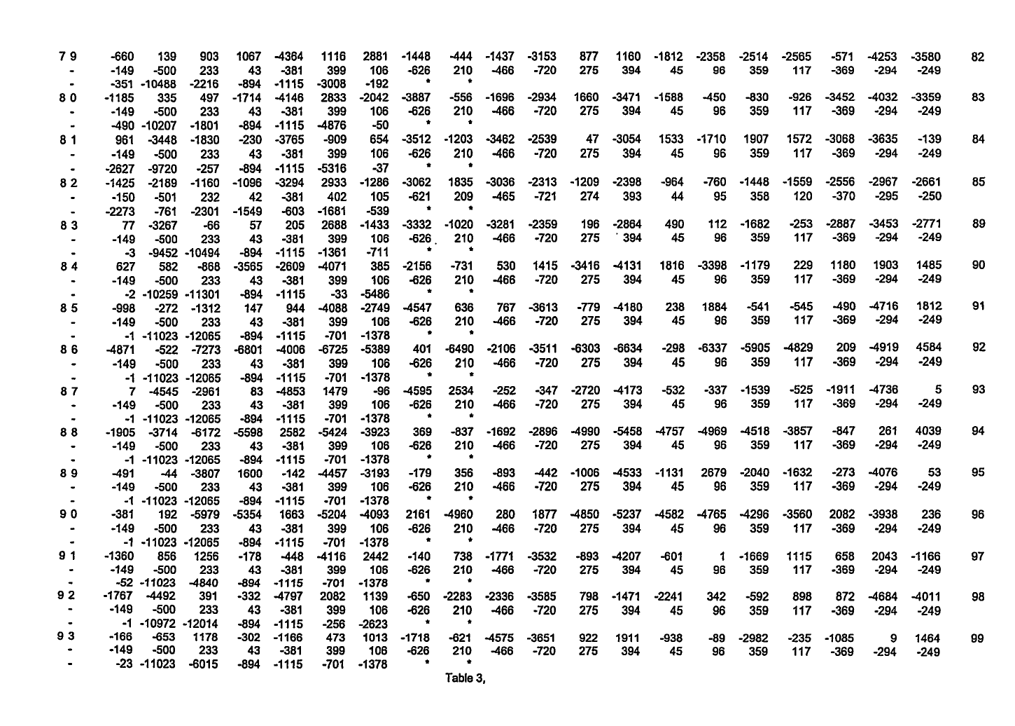

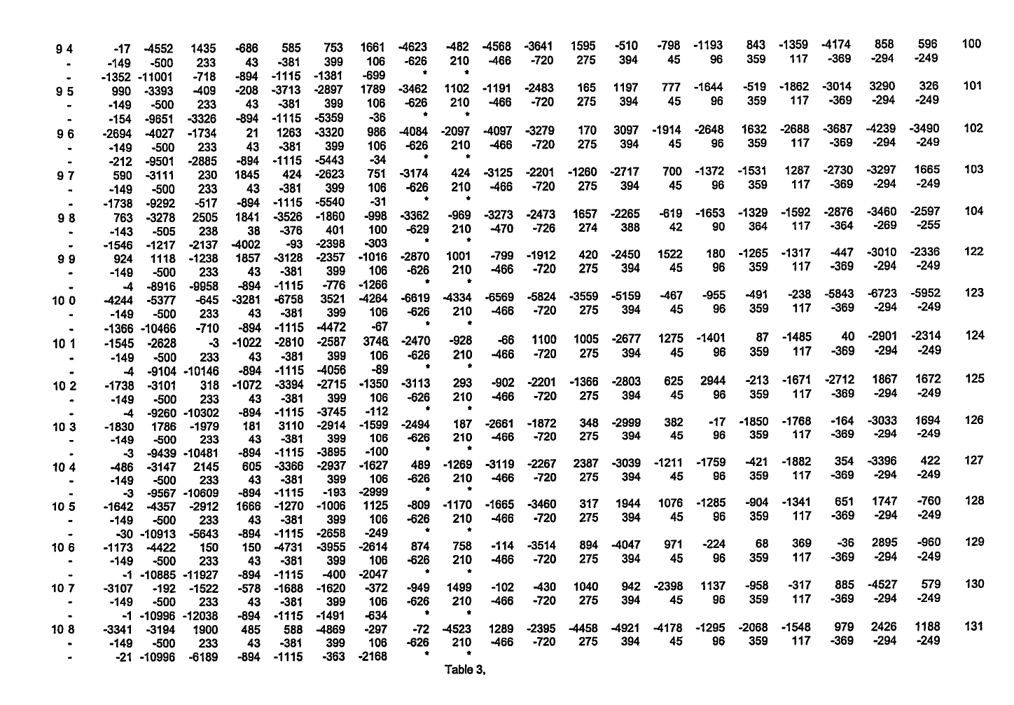

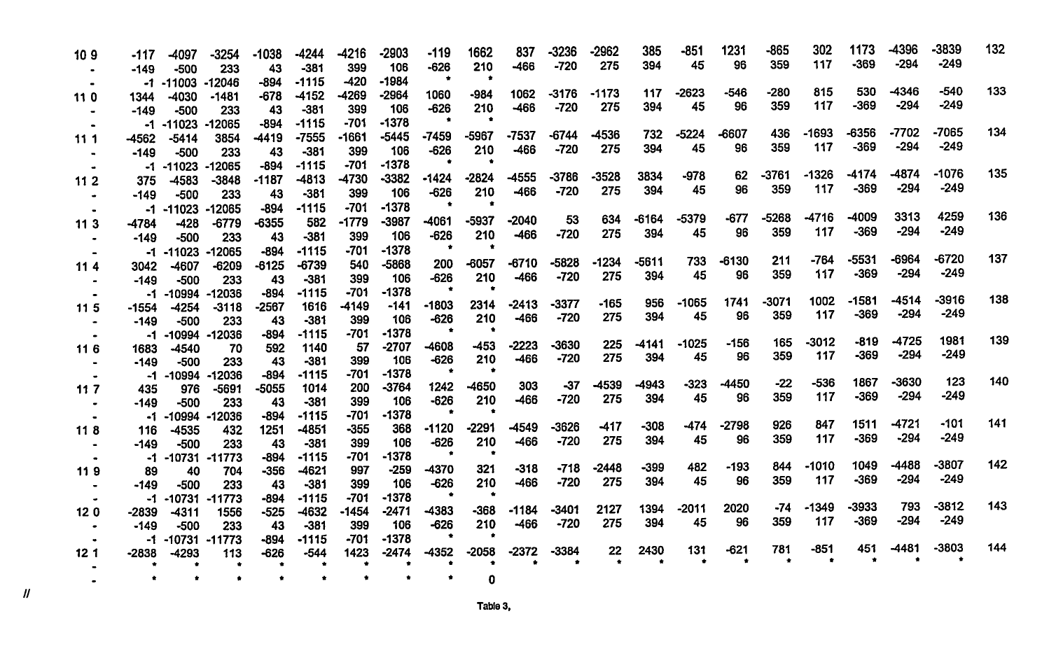

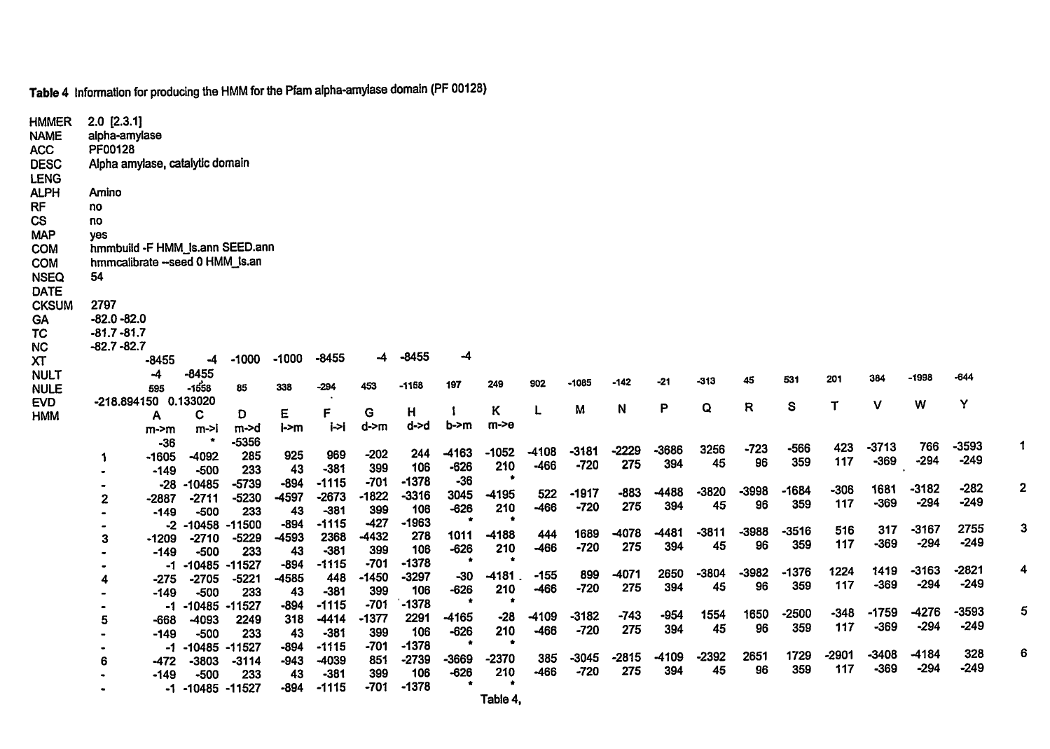

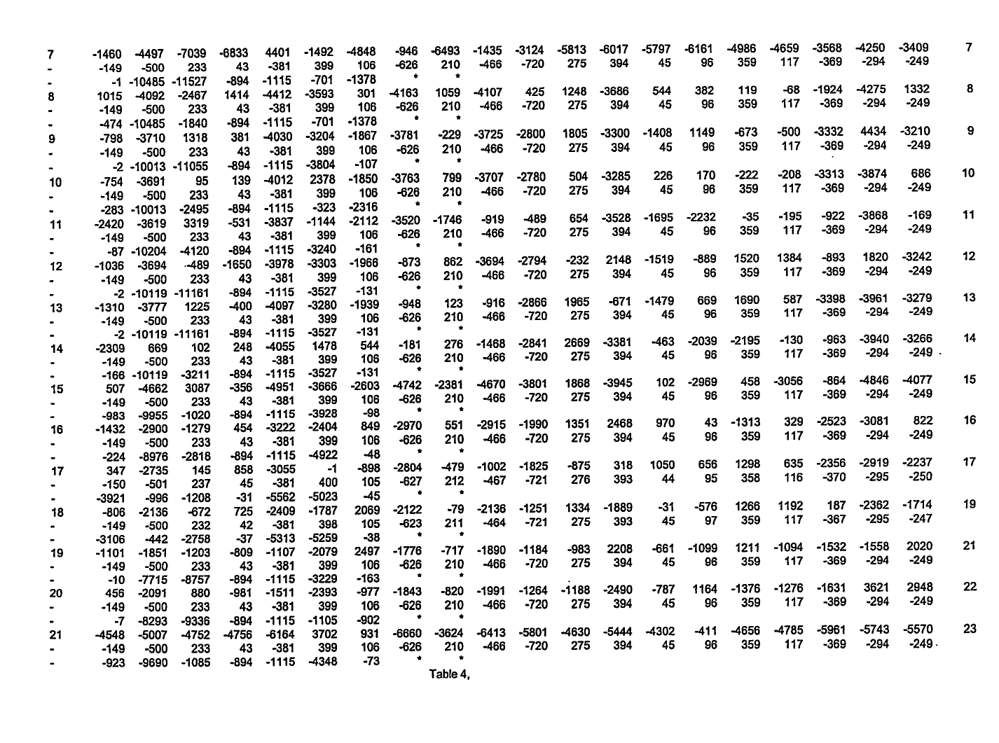

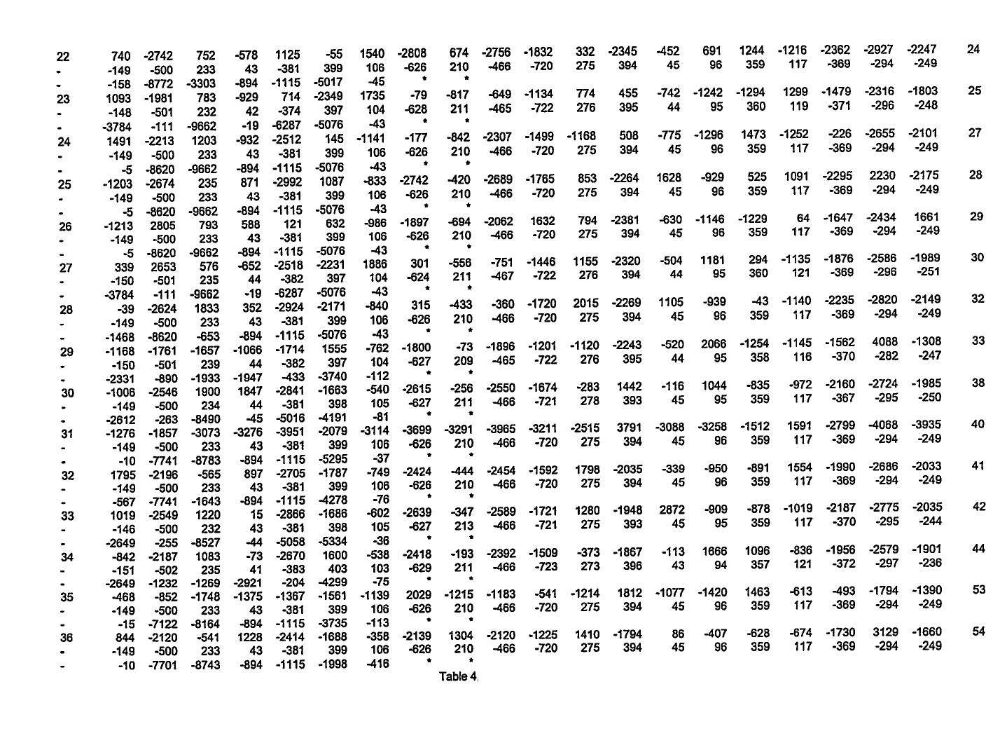

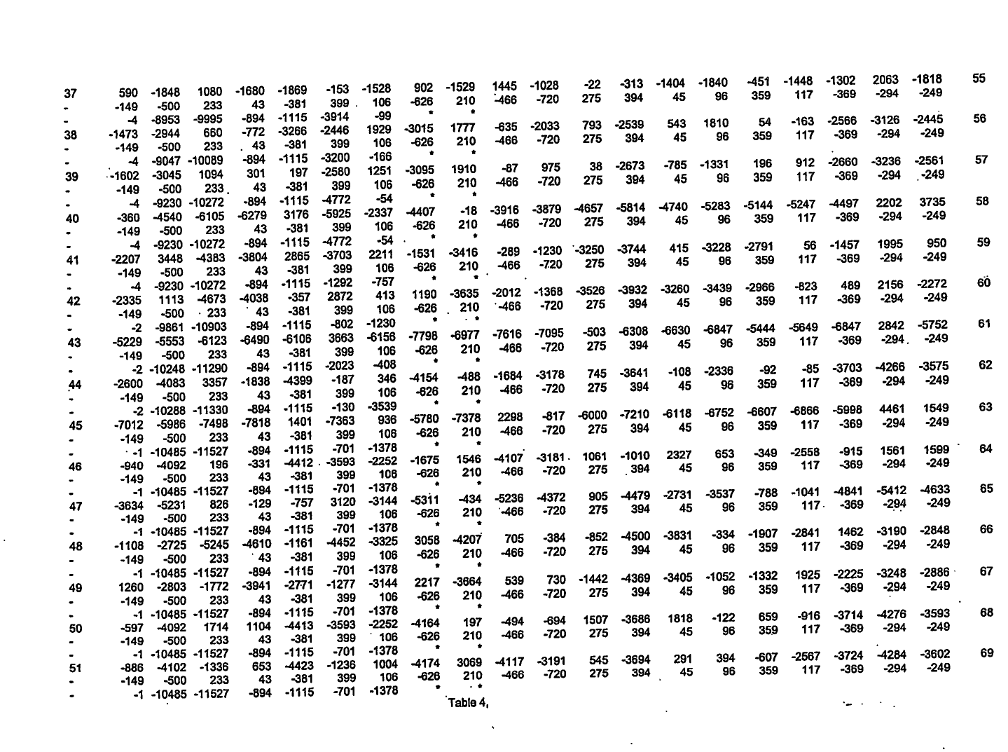

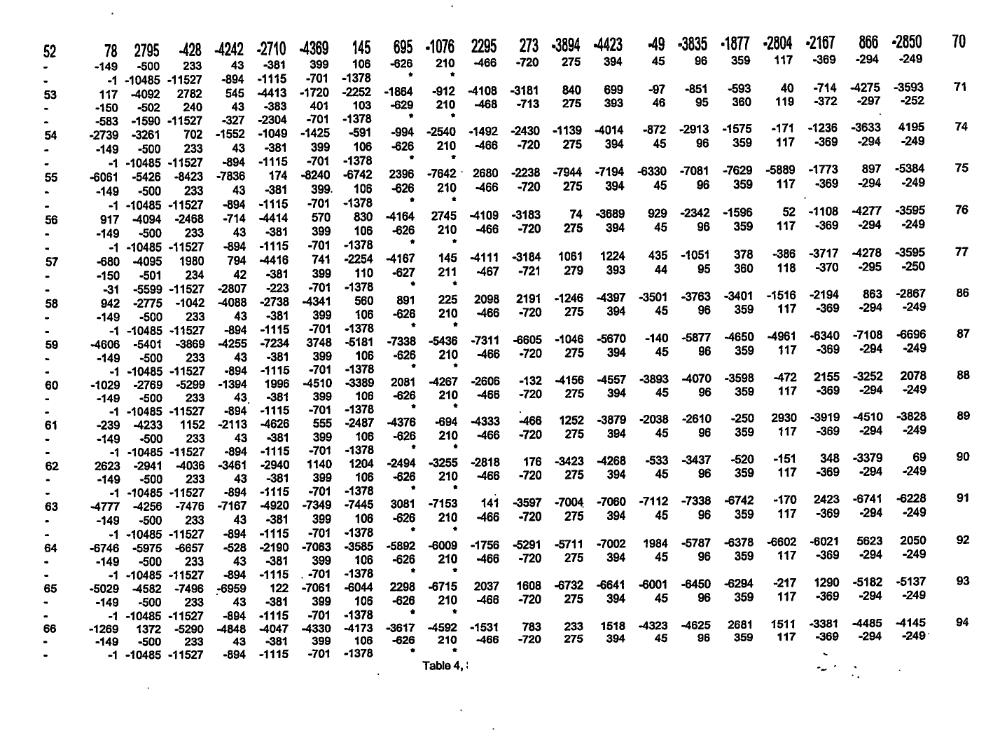

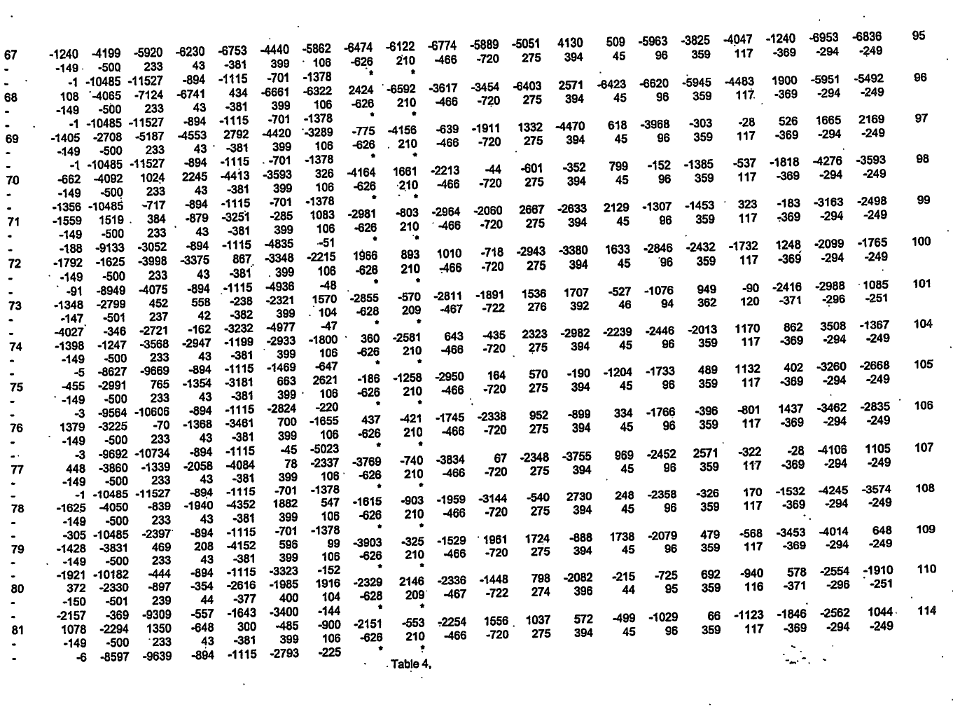

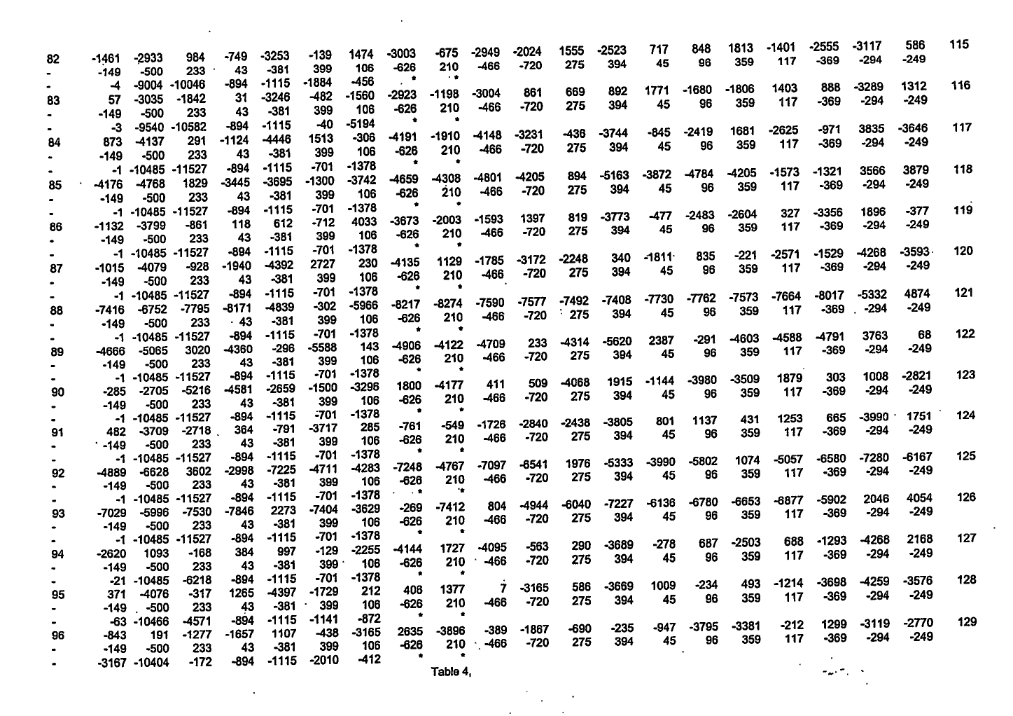

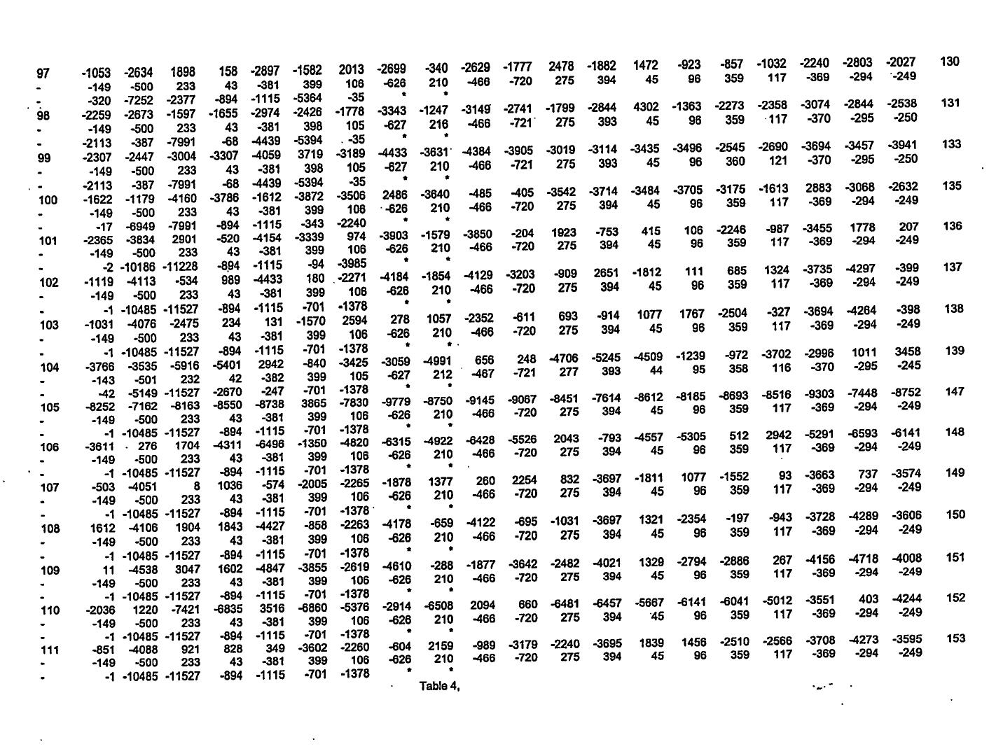

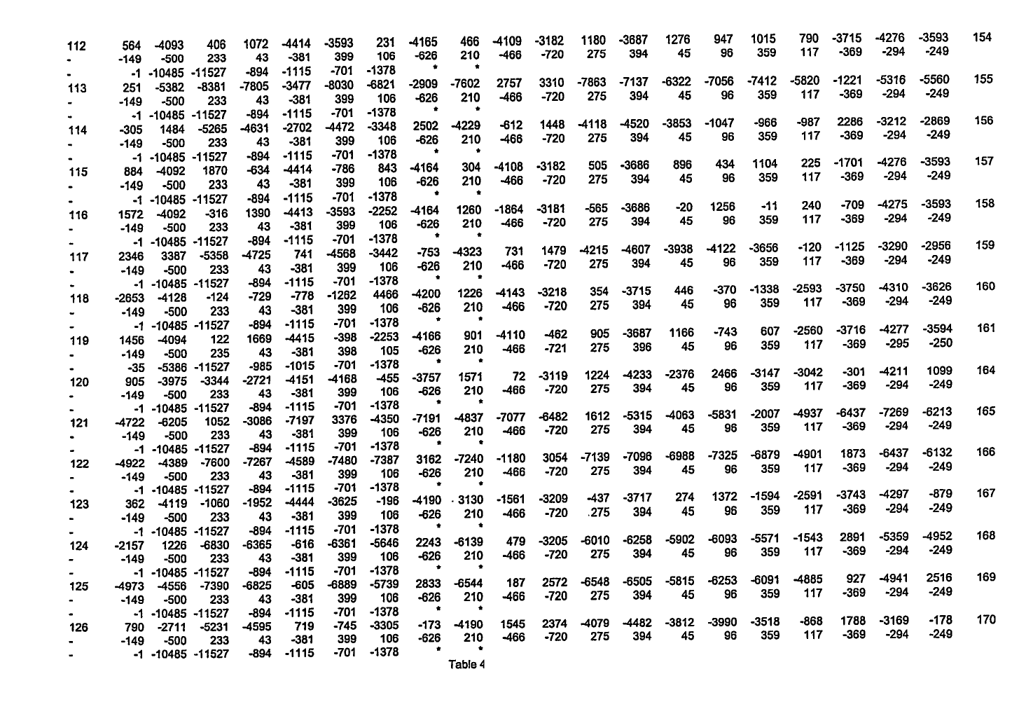

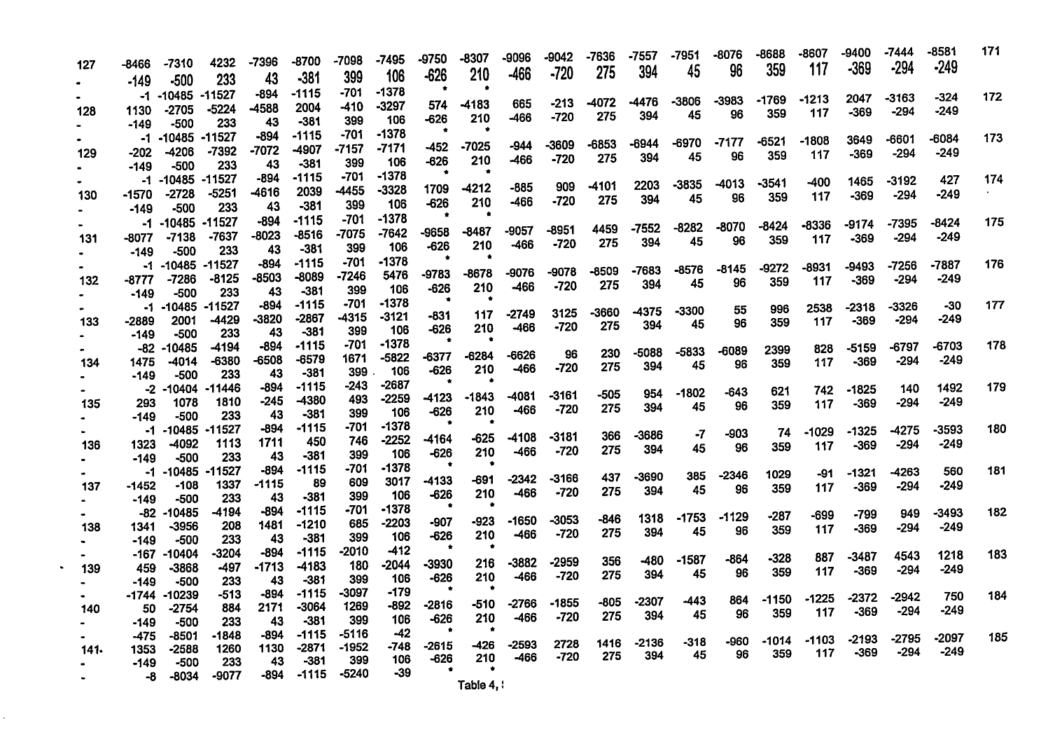

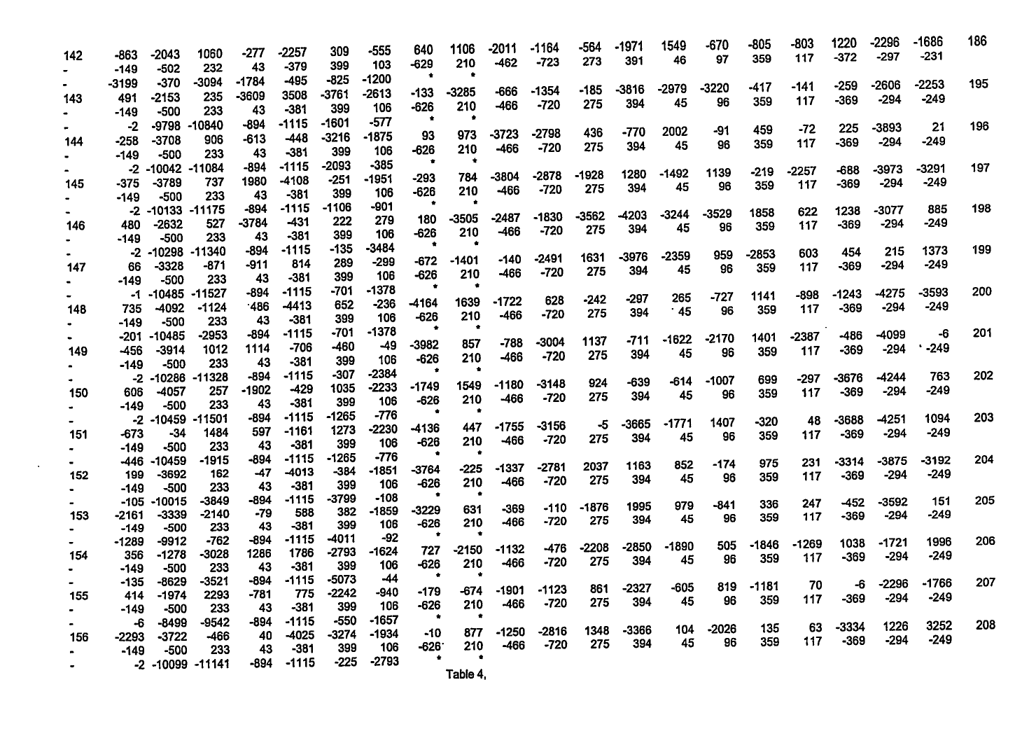

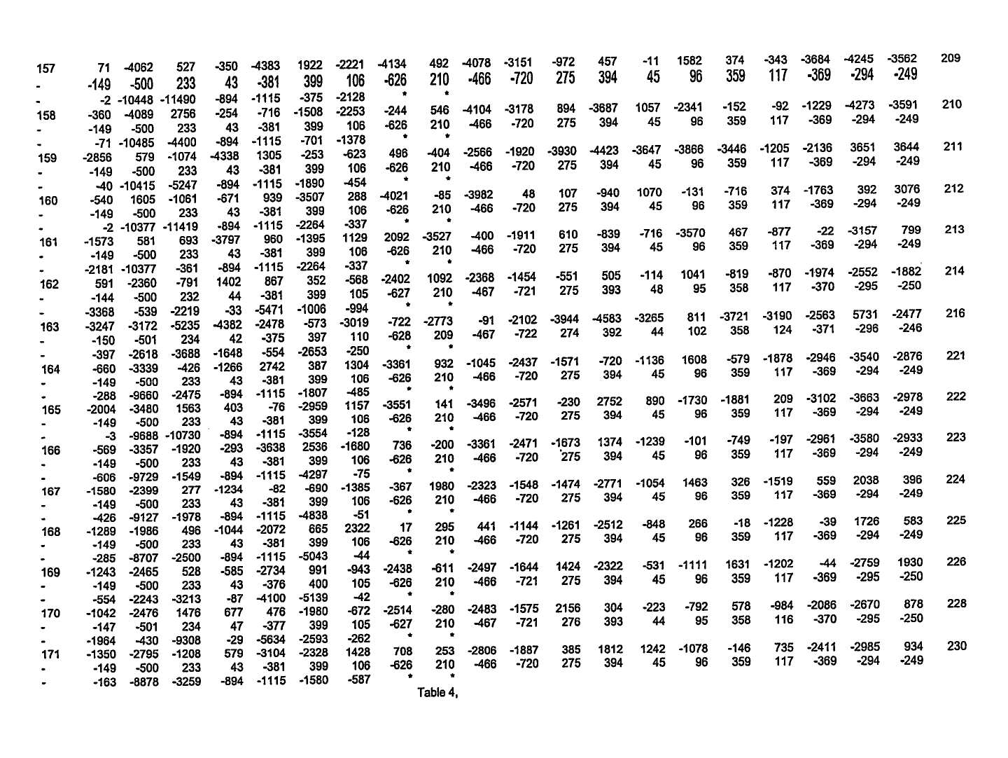

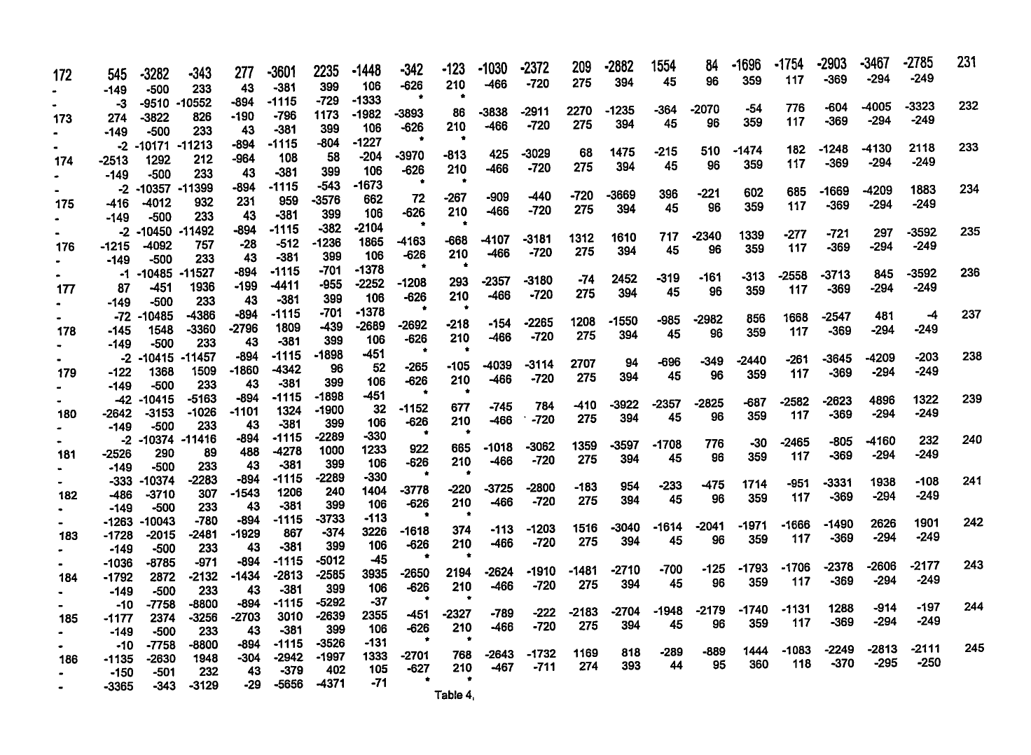

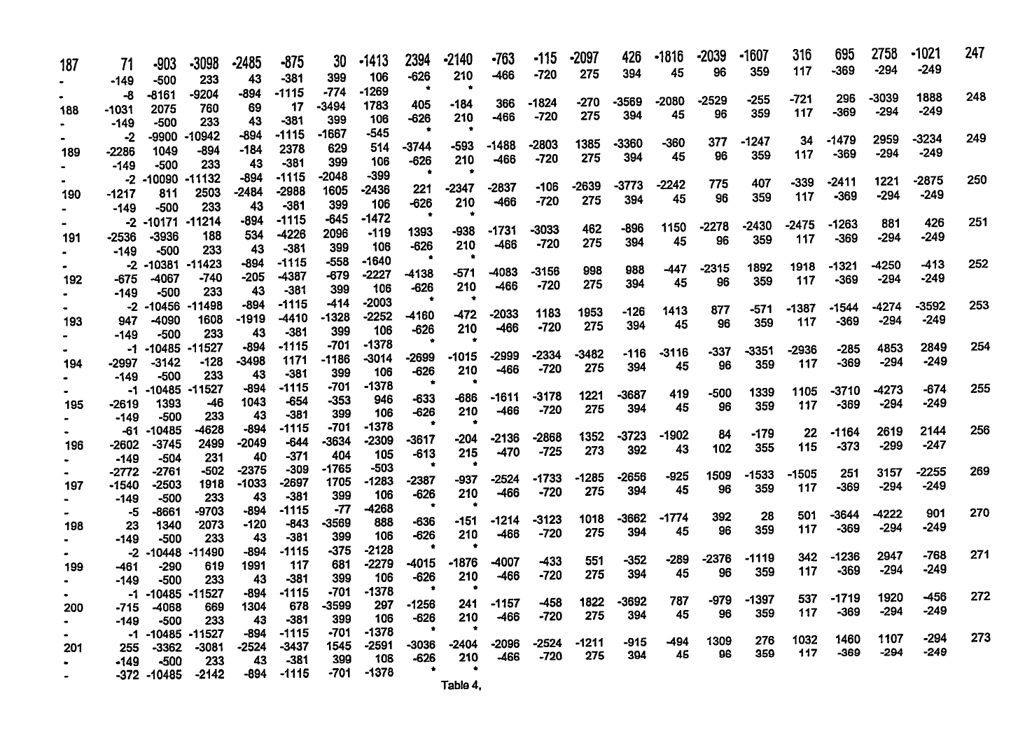

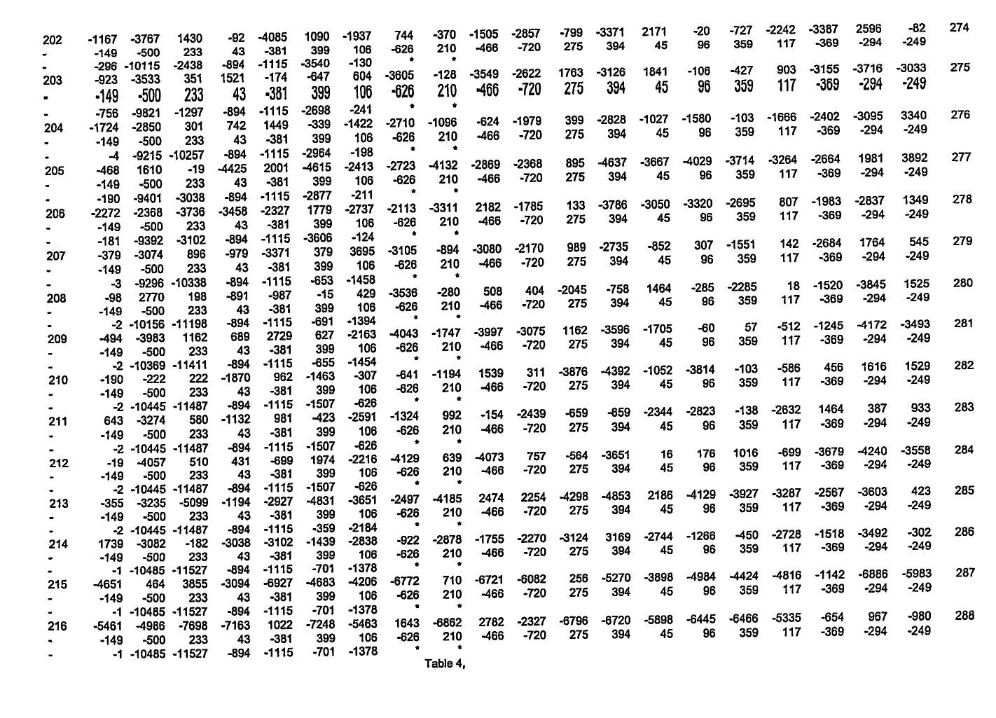

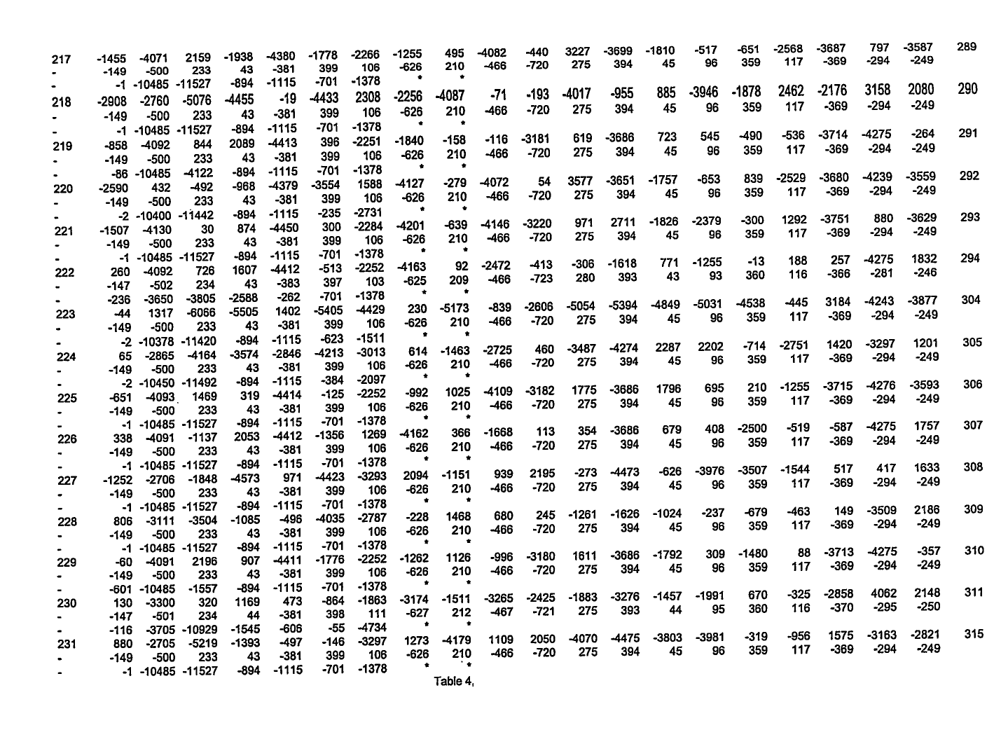

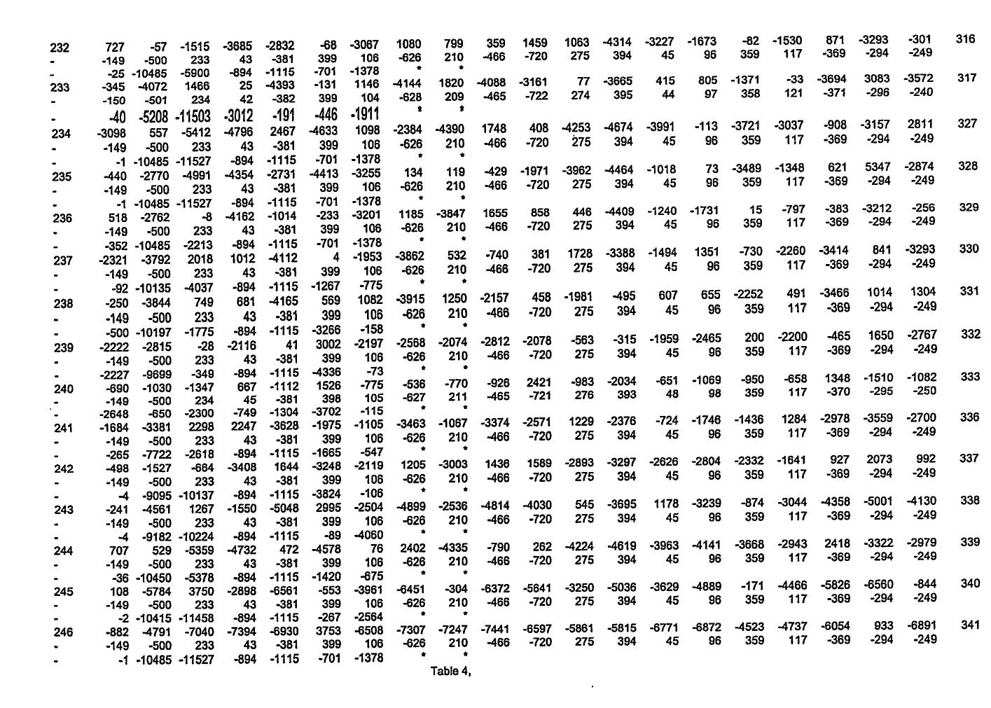

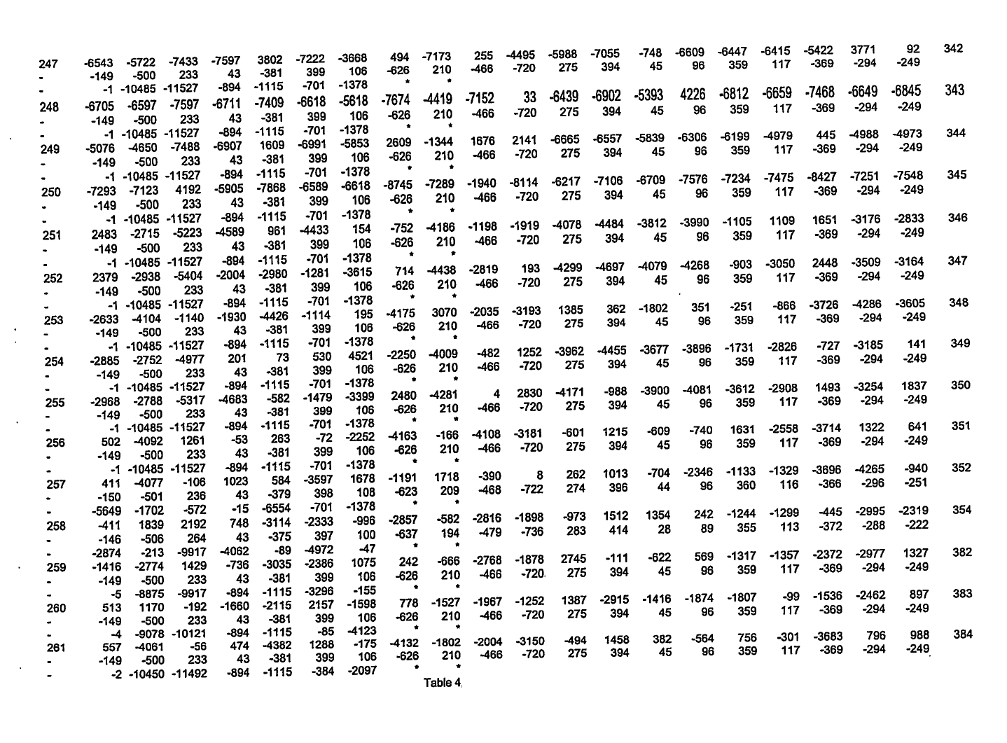

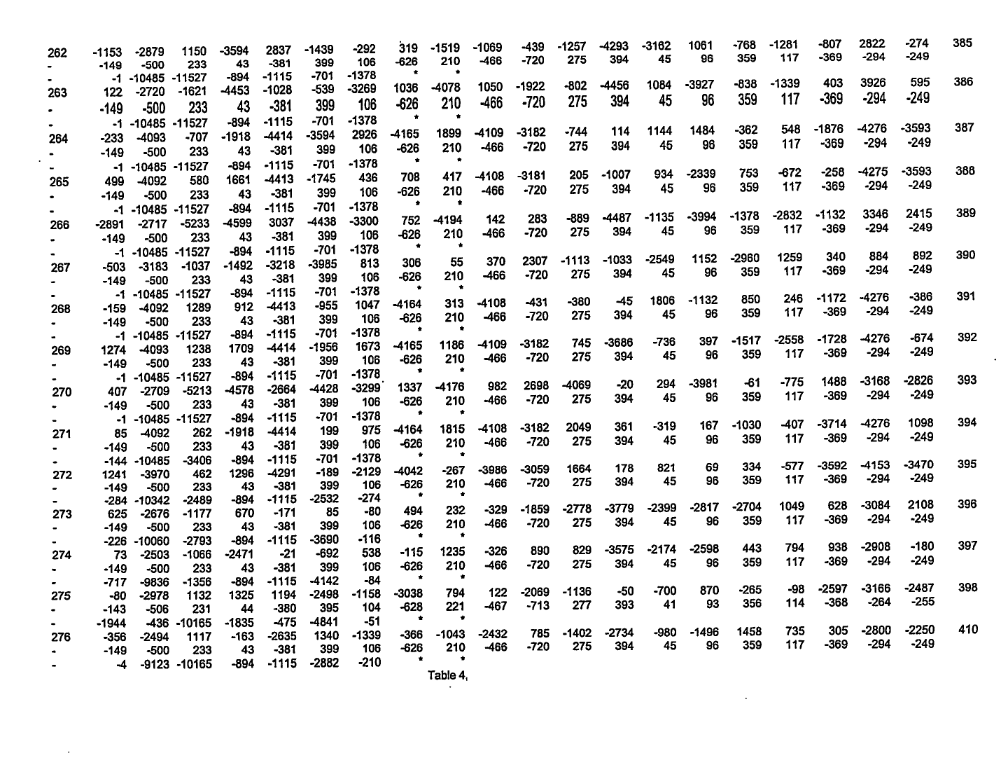

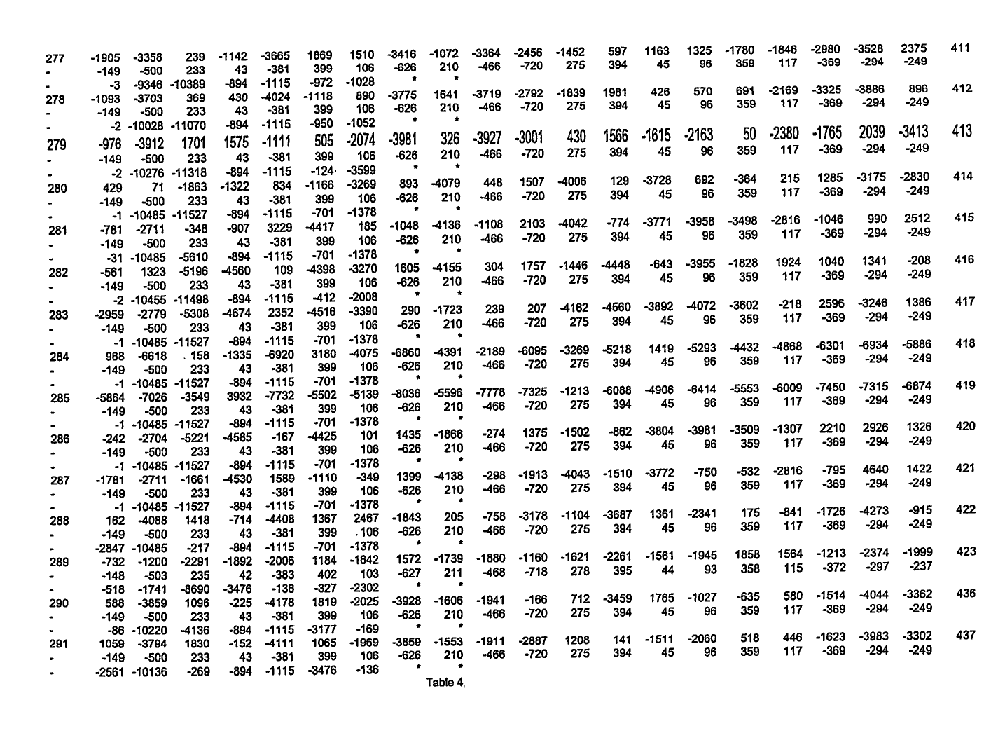

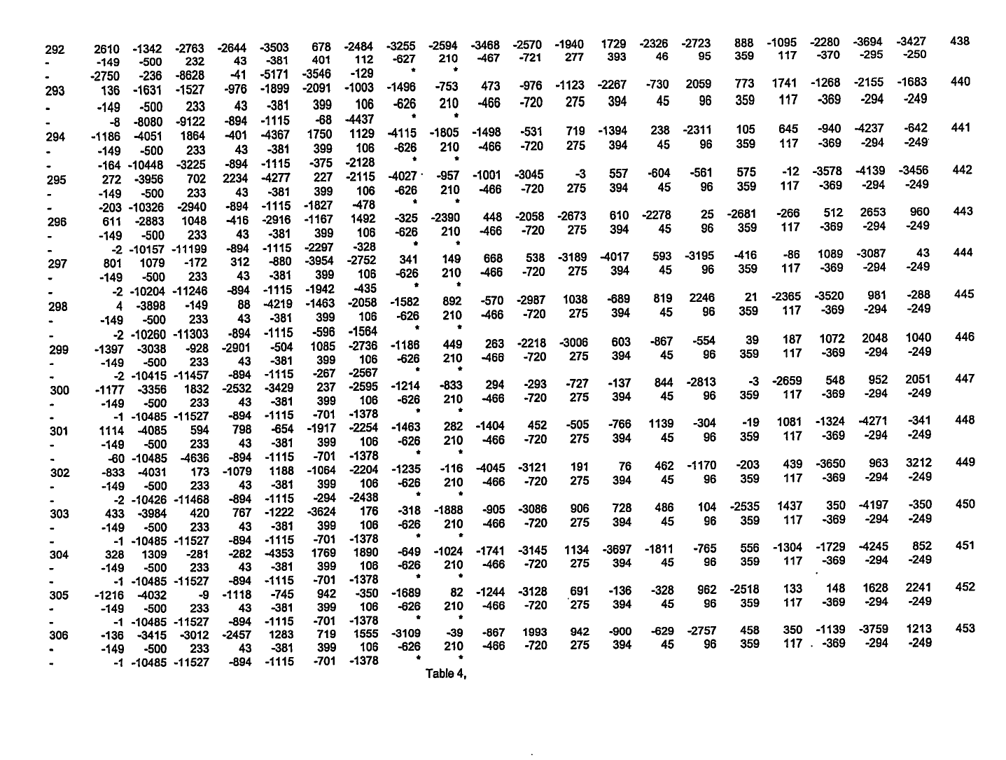

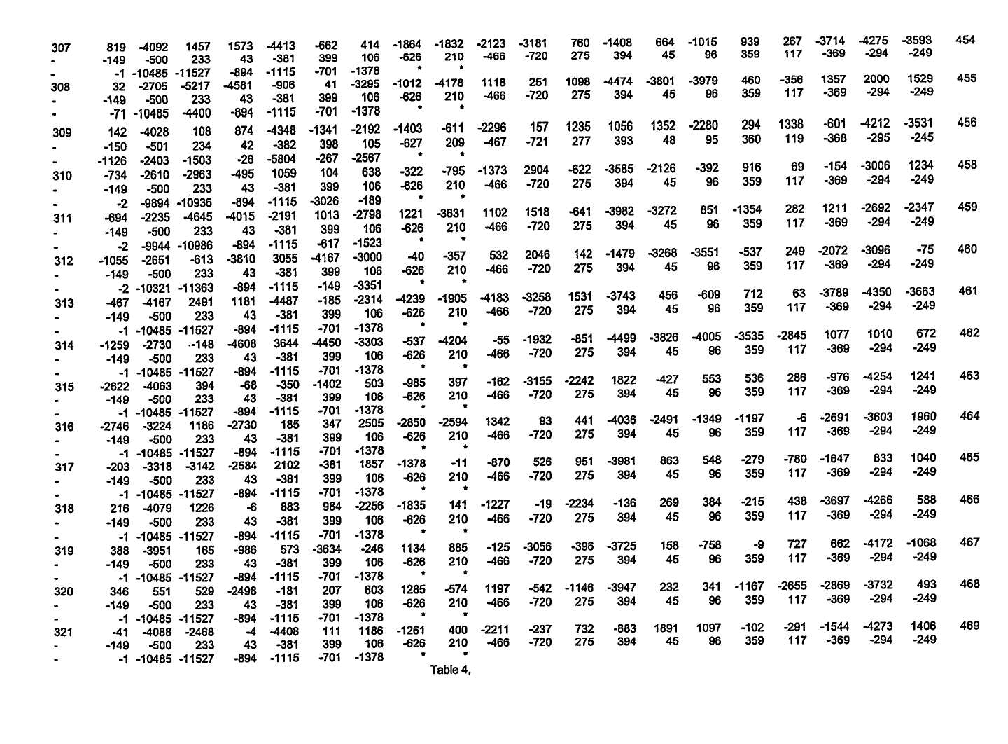

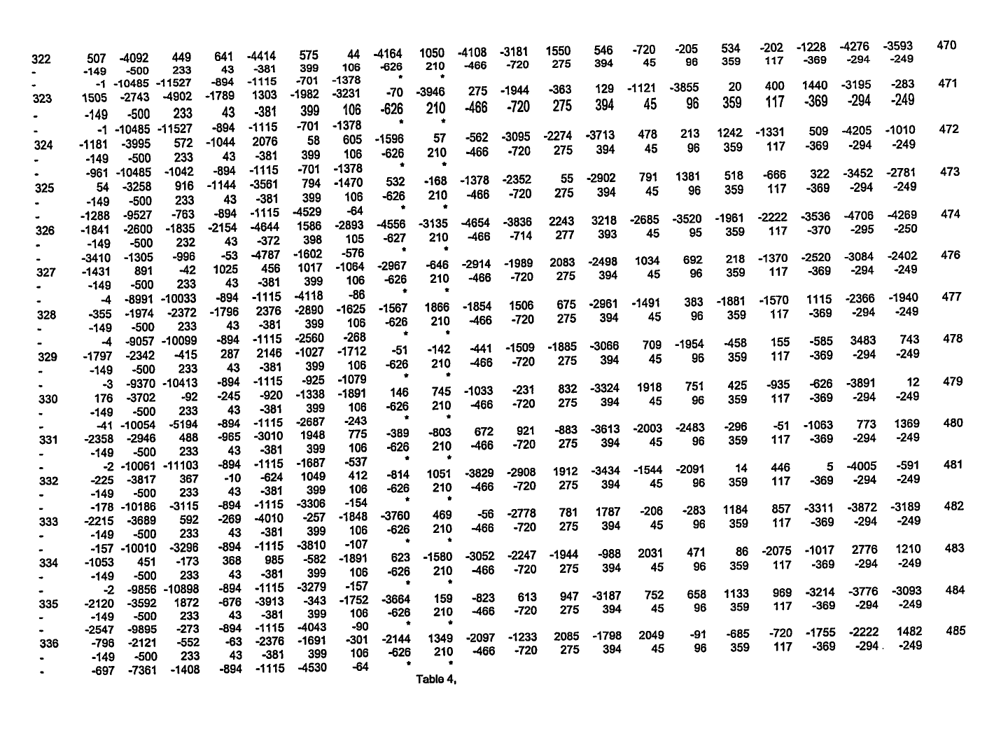

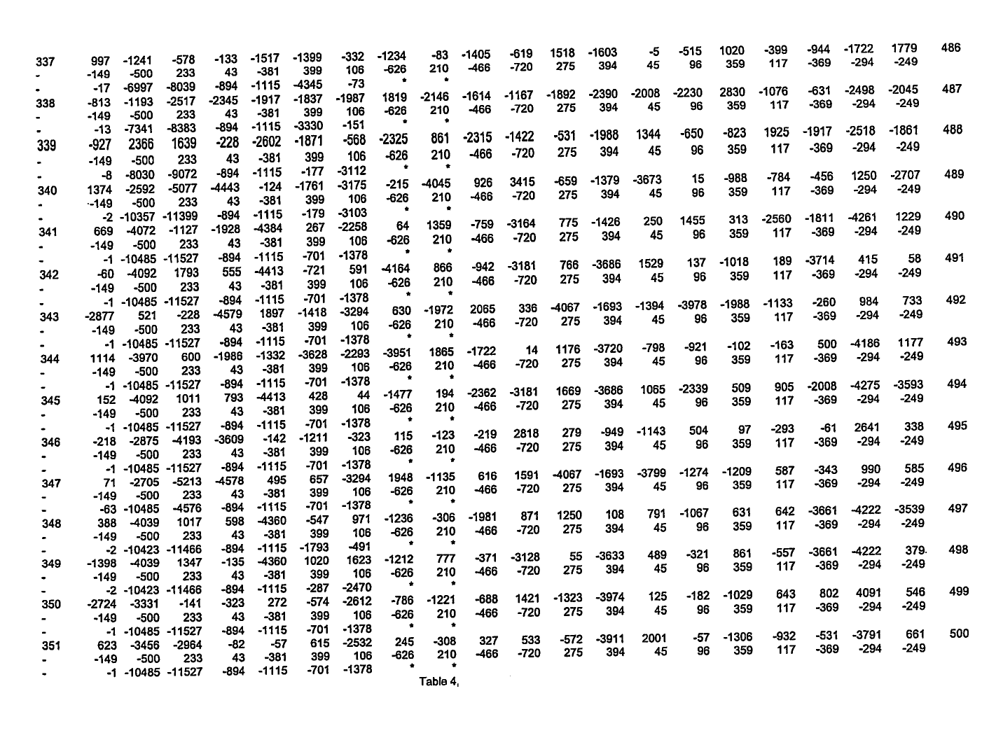

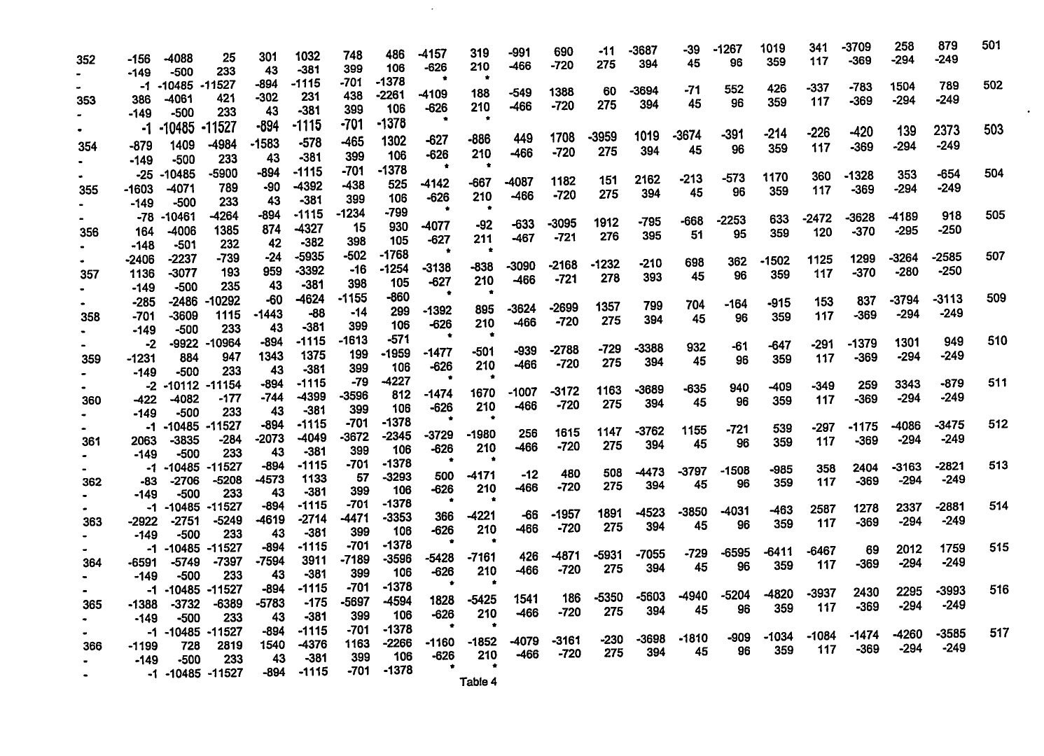

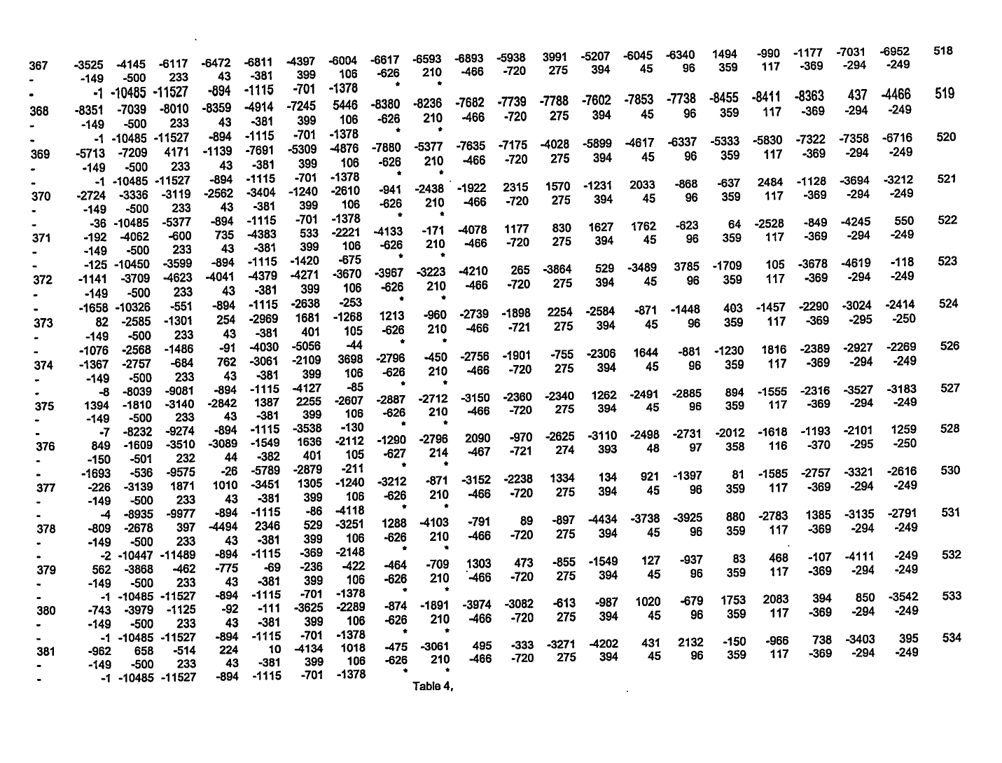

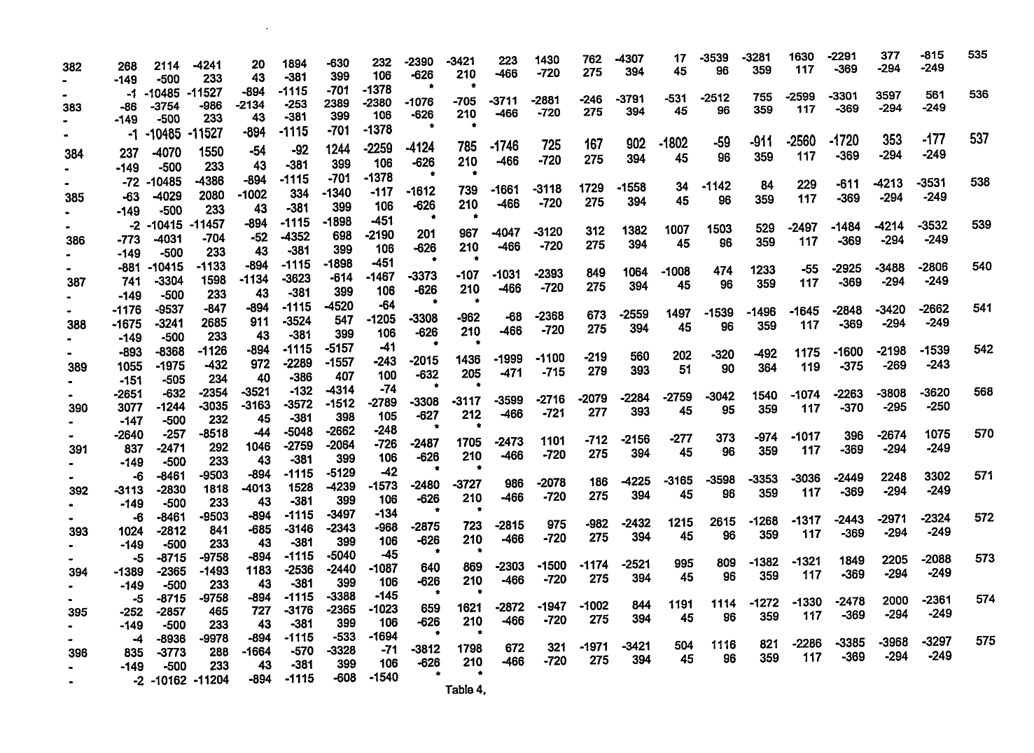

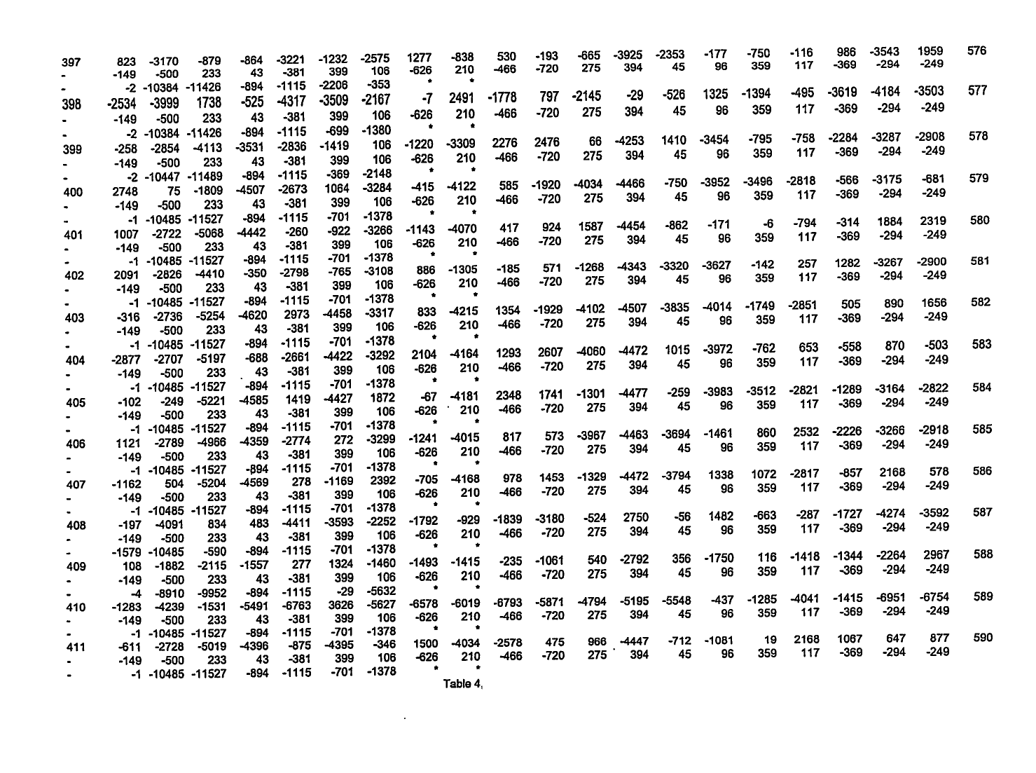

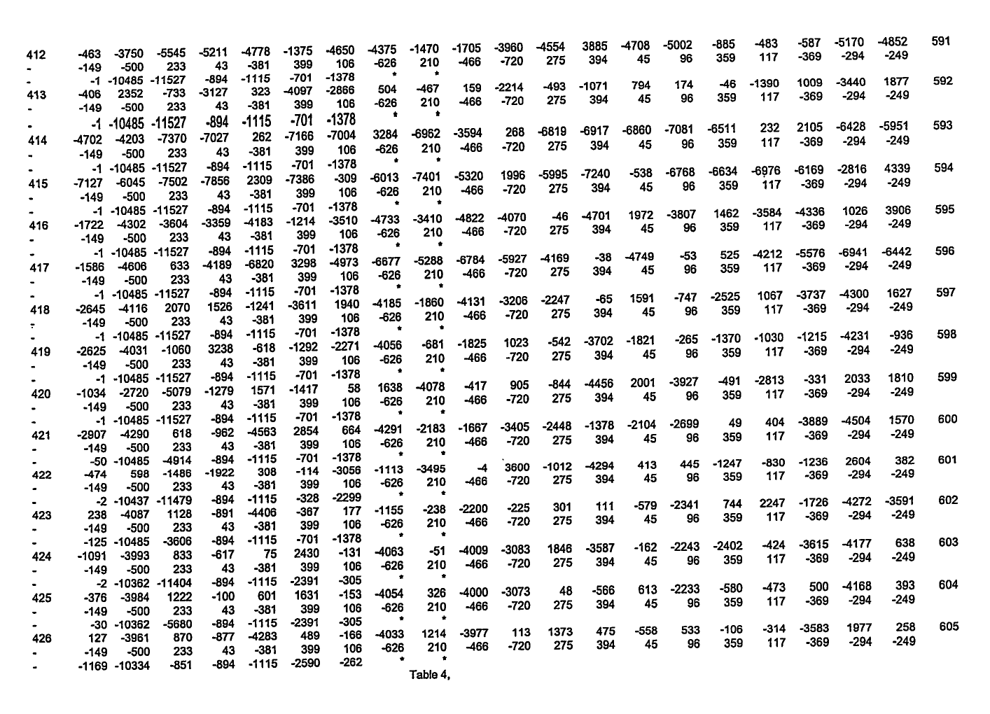

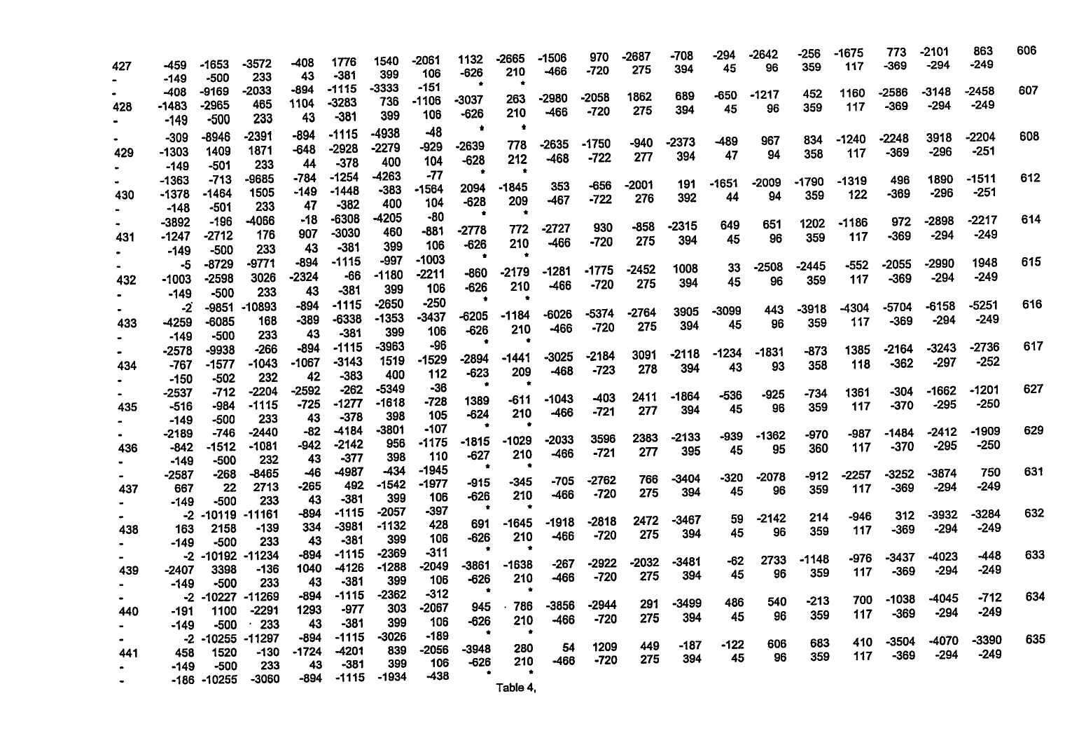

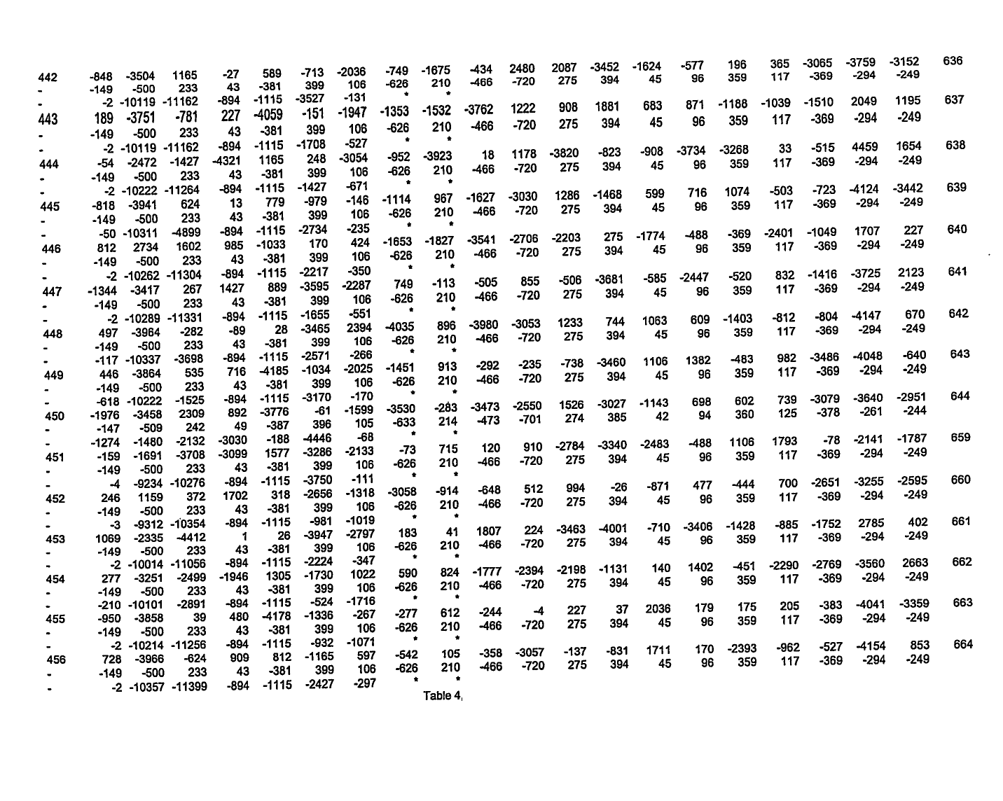

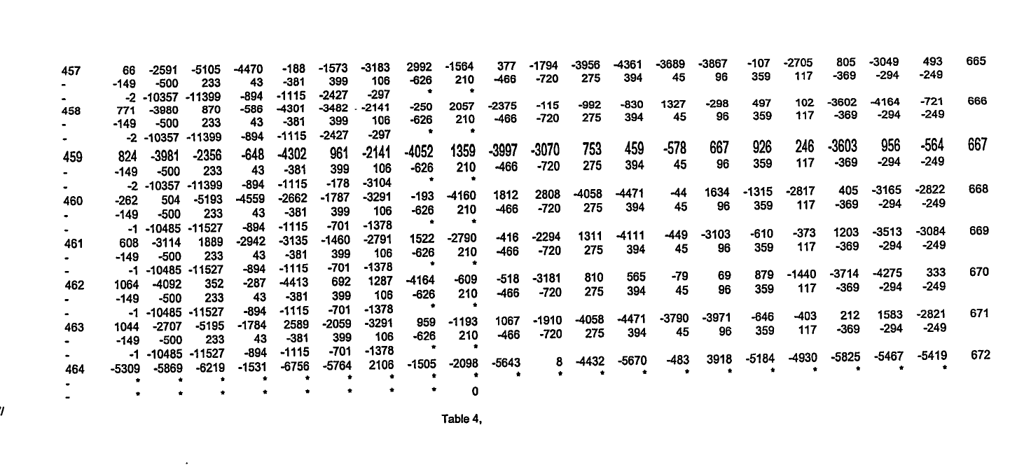

Relative expression levels of the respective transcripts were calculated as described in M. W. Pfaffl (2001, “A new mathematical model for relative quantification in real-time RT-PCR”, Nucleic Acids Res. 29(9):e45) Therefore the expression levels of transcripts coding potato branching enzyme Class 3 were calculated as relative amounts compared to the expression levels of transcripts coding S-adenosylmethionindecarboxylase, wich is highly and consistently expressed during seed development in rice. The relative expression levels calculated as just described, are shown in FIG. 1.

1. A genetically modified plant cell having an increased activity of at least one Class 3 branching enzyme in comparison with corresponding wild type plant cells that have not been genetically modified.

2. The genetically modified plant cell according to claim 1, wherein the genetically modified plant cell comprises at least one foreign nucleic acid molecule that has been introduced into the genome of the plant cell.

3. The genetically modified plant cell according to claim 2, wherein the foreign nucleic acid molecule codes a Class 3 branching enzyme.

4. The genetically modified plant cell according to claim 3, wherein the said foreign nucleic acid molecule is
a) a nucleic acid molecule, which codes a protein with the amino acid sequence given under Seq ID NO 4;
b) a nucleic acid molecule, which codes a protein, the amino acid sequence of which has an identity of at least 50% with the amino acid sequence given under SEQ ID NO: 4;
c) a nucleic acid molecule, which includes the nucleotide sequence of Seq ID NO 3 or a complementary sequence;
d) a nucleic acid molecule, the nucleic acid sequence of which has an identity of at least 50% with the nucleic acid sequences described under a) or c);
e) a nucleic acid molecule, which hybridizes with at least one strand of the nucleic acid molecules described under a) or c) under stringent conditions;
f) a nucleic acid molecule, the nucleotide sequence of which deviates from the sequence of the nucleic acid molecules identified under a), b), c), d), e) or f) due to the degeneration of the genetic code; or
g) a nucleic acid molecule, which represents fragments, allelic variants and/or derivatives of the nucleic acid molecules identified under a), b), c), d), e) or f).

5. The genetically modified plant cell according to claim 2, wherein said foreign nucleic acid molecule is
a) a T-DNA molecule, which leads to an increase in the expression of a Class 3 branching enzyme gene due to integration in the vegetable genome;
b) a DNA molecule, which contains transposons, which leads to an increase in the expression of a Class 3 branching enzyme gene due to integration in the vegetable genome;
c) a DNA molecule, which codes a Class 3 branching enzyme and which are linked with regulatory sequences, which guarantee transcription in vegetable cells and lead to an increase in Class 3 branching enzyme activity in the cell; or
d) a nucleic acid molecule introduced by means of in vivo mutagenesis, which leads to a mutation or an insertion of a heterologous sequence in at least one endogenous gene coding a Class 3 branching enzyme, wherein mutation or insertion effects an increase in the expression of a gene coding a Class 3 branching enzyme.

6. A plant cell according to claim 1, which synthesizes a modified starch in comparison with corresponding wild type plant cells that have not been genetically modified.

7. A plant containing plant cells according to claim 1.

8. A plant according to claim 7, which is a starch-storing plant.

9. A plant according to claim 7, which is a maize, rice, wheat, rye, oat, barley, cassava, potato, sago, mung bean, pea or sorghum plant.

10. The plant according to claim 9, which is a potato plant.

11. Propagation material of plants according to claim 7.

12. Harvestable plant parts of plants according to claim 7.

13. A method for the manufacture of a genetically modified plant according to claim 7, comprising
a) genetically modifying a plant cell, whereby the genetic modification leads to an increase in the activity of a Class 3 vegetable branching enzyme in comparison with corresponding wild type plant cells that have not been genetically modified;
b) regenerating a plant from plant cells from Step a); and
c) if necessary, producing further plants with the help of the plants according to Step b).

14. The method according to claim 13, wherein the genetic modification comprises introduction of a foreign nucleic acid molecule into the genome of the plant.

15. The method according to claim 14, wherein the said foreign nucleic acid molecule is
a) a nucleic acid molecule, which codes a protein with the amino acid sequence of Seq ID NO 4;
b) a nucleic acid molecule, which codes a protein, the amino acid sequence of which has an identity of at least 50% with the amino acid sequence of SEQ ID NO: 4;
c) a nucleic acid molecule, which includes the nucleotide sequence shown under Seq ID NO 3 or a complementary sequence;
d) a nucleic acid molecule, the nucleic acid sequence of which has an identity of at least 50% with the nucleic acid sequences described under a) or c);
e) a nucleic acid molecule, which hybridizes with at least one strand of the nucleic acid molecules described under a) or c) under stringent conditions;
f) a nucleic acid molecule, the nucleotide sequence of which deviates from the sequence of the nucleic acid molecules identified under a), b), c), d), e) or f) due to the degeneration of the genetic code; or
g) a nucleic acid molecule, which represents fragments, allelic variants and/or derivatives of the nucleic acid molecules identified under a), b), c), d), e) or f).

16. The method according to claim 14, wherein said foreign nucleic acid molecule is
a) a T-DNA molecule, which leads to an increase in the expression of a Class 3 branching enzyme gene due to integration in the vegetable genome;
b) a DNA molecule, which contains transposons, which leads to an increase in the expression of a Class 3 branching enzyme gene due to integration in the vegetable genome;
c) a DNA molecule, which codes a Class 3 branching enzyme and which are linked with regulatory sequences, for transcription in vegetable cells and leads to an increase in Class 3 branching enzyme activity in the cell; or
d) a Nucleic acid molecule introduced by means of in vivo mutagenesis, which leads to a mutation or an insertion of a heterologous sequence in at least one endogenous gene coding a Class 3 branching enzyme, wherein the mutation or insertion effects an increase in the expression of a gene coding a Class 3 branching enzyme.

17. The method according to claim 13, wherein the genetically modified plant synthesizes a modified starch in comparison with corresponding wild type plants that have not been genetically modified.

18. A modified starch obtainable from a genetically modified plant according to claim 7, from propagation material according to claim 11, or from harvestable plant parts according to claim 12.

19. A method for the manufacture of a modified starch including the step of extracting the starch from a plant cell according to claim 1.

20. A method for the manufacture of a modified starch including the step of extracting the starch from a plant according to claim 7.

21. A method for the manufacture of a modified starch including the step of extracting the starch from harvestable plant parts according to claim 12.

22. A method for the manufacture of a derived starch, wherein modified starch according to claim 18 or obtainable by the method according to claim 19 is derived.

23. (canceled)

24. A modified starch obtainable by the method according to claim 19.

25. Derived starch obtainable by the method according to claim 22.

26. (canceled)


SEQUENCE LISTING (6 sequences)

SEQ ID NO: 1 - DNA, 1004 bp, Solanum tuberosum
tcaaactagtcacaaccagtccatttctggaggtcgttccttcgcagaaatactgattggtaactccttggggaaatcctccatatcacaagagtcattacttagaggctgctcgttacacaagatgatcagattaattacatctacaattggtggtcatgcatacctcaacttcatgggcaatgaatttggtcacccaaagagagtagagtttccaatgtcaagcaacaatttctccttttcactggctaaccgtcgctgggatctattggaagatgttgtacattatcaattgttctcatttgataagggtatgatggacttggataaaaatgggagaattttgtccagaggtcttgccaacattcaccatgtcaatgatactaccatggtgatttcttacttgagaggtcccaatctctttgtgttcaactttcatcctgtcaattcatatgaaagatacattataggtgtggaagaagctggagagtatcaagtcacattaaatacagatgaaaacaagtatggtggtagaggactacttggccatgatcagaatattcaaagaaccattagtagaagagctgatggaatgagattttgcttggaagtgcctctgccaagtagaagtgctcaggtctacaagttgacccgaattctaagagcatgatcactctagtaatcaaagtgcctcatatgatgacacaaaaggaaaggttctacattgcccttacactgatcaatattgacacctttccgaggtgagtttctgtgattcttgagcagactgttggctagtcaattatcatgaacttttgccttcagcatccggatagtcgcttctcctgtgcaatgagggcatggacgaatttttttttggcttgtcatgggggtcataagcatccgccagattaagatttcacaggcctcgagtaaaaccatcacttactttaaggatacacaaacacaccaacggggtgcaggctctgataccttctaaagtg

SEQ ID NO: 2 - DNA, 2096 bp, Solanum tuberosum
aacaatgctctctctgtcggattcaattcgaatttcttcaccattgagcgattctcgtcttagttttctatctcaaaccggaagcagaaccagtcgccagcttaaatttgttcgcagccgccgggctcgagtttcgaggtgtagatgctcagcaacggagcaaccgccaccgcaacgacggaagcaacgaccggagaagtacaaacagtcggaggaagggaaaggaatcgatcctgttggatttctcagcaaatacggcattactcataaagcgtttgctcaatttcttcgtgaaagatataaatcattgaaggacttgaaggatgaaatattgactcgtcatttcagtctcaaggagatgtctactgggtatgaattaatgggtatgcatcgcaacatacaacatcgagtggatttcttggaatgggctccaggtgctcgctactgtgctctgattggtgacttcaatgggtggtcaacaactggtaactgtgccagagagggtcattttggtcatgacgattatgggtattggtttattattcttgaagataaattacgtgaaggagaagaacctgataaattgtattttcaacagtacaattatgcggaggactatggtaaaggtgacacgggtattaccgtcgaggaaatctttaaaaaagcaaatgatgagtattgggaacctggagaagatcgcttcattaaatcacgttatgaggtggcagcaaagttatatgaggaaatgttcggaccaaatggacctcaaacagaagaggaactagaagcaatgcctgatgcagctacacgatacaaaacttggaaagagcaacaaaaagaggatccggcaagcaatttgccatcgtatgatgtggtagatagtggaaaagaatatgatatttacaatattataggtgatcctgaatcgtttaagaaatttcgtatgaaacagcctcctattgcttactggttagaaactaaaaagggaaggaaaggctggttacagaaatatatgcctgctttacctcatggaagcaaatacagggtgtattttaacacaccaaatgggcctcttgaacgagttcctgcgtgggccaattttgtcattccagatgcaggcgggatggcattagcagtccattgggaaccacctcctgaatatgcttataaatggaaacacaagctaccagtcaagcctaagtccttgcgcatatatgaatgtcatgttggcatctctggccaggaaccaaaagtttcatctttcaatgattttattagcaaggtccttccgcatgtaaaagaagctggatacaatgcaatacaaattattggagttgttgagcacaaggattatttcactgttggatatagagtgaccaatttttatgctgttagtagccgttatggcacaccggatgacttcaagcgcttggttgatgaagcacatgggcttggactgcttgtctttttggagattgtgcactcttatgcagcagcagatgaaatggttgggttatctctttttgatggagcaaatgattgctatttccacactggtaaacgtggacaccacaaattctggggcacacggatgttcaaatatggagatcttgatgttctgcactttcttctttcaaatctgaactggtgggtggaggagtatcatgtcgatggcttccattttcattcgctctcgtccatgttgtatacgcataatggatttgcttcatttactggtgacatggatgaatactgtaaccaatatgttgacaaggaggccttattgtacctcatattagcaaatgaagtattacatgctcttcatcctaatgtgatcacgattgctgaggatgcaactctgtatcctggactctgcgatccaacatctcaaggtggactgggctttgattattttgccaatctttctgcctcagagatgtggcttgcattacttgaaaatactcctgatcatgaatggtgcatgagtaagattgttagcacattagtgggcgatagacaaaatactgataaaatgcttttgtatgcagaaaatcacaaccagtccatttctggaggtcgttccttcgcagaaatac

SEQ ID NO: 3 - DNA, 3204 bp, Solanum tuberosum
  CDS (99)..(2804)
gaattgtaatacgactcactatagggcgaattgggccctctagatgcatgctcgagcggccgccagtgtgatggatatctgcagaattcggcttaacaatgctctctctgtcggattcaattcgaatttcttcaccattgagcgattctcgtcttagttttctatctcaaaccggaagcagaaccagtcgccagcttaaatttgttcgcagccgccgggctcgagtttcgaggtgtagatgctcagcaacggagcaaccgccaccgcaacgacggaagcaacgaccggagaagtacaaacagtcggaggaagagaaaggaatcgatcctgttggatttctcagcaaatacggcattactcataaagcgtttgctcaatttcttcgtgaaagatataaatcattgaaggacttgaaggatgaaatattgactcgtcatttcagtctcaaggagatgtctactgggtatgaattaatgggtatgcatcgcaacatacaacatcgagtggatttcttggaatgggctccaggtgctcgctactgtgctctgattggtgacttcaatgggtggtcaacaactggtaactgtgccagagagggtcattttggtcatgacgattatgggtattggtttattattcttgaagataaattacgtgaaggagaagaacctgataaattgtattttcaacagtacaattatgcggaggactatgataaaggtgacacgggtattaccgtcgaggaaatctttaaaaaagcaaatgatgagtattgggaacctggagaagatcgcttcattaaatcacgttatgaggtggcagcaaagttatatgaggaaatgttcggaccaaatggacctcaaacagaagaggaactagaagcaatgcctgatgcagctacacgatacaaaacttggaaagagcaacaaaaaaaggatccggcaagcaatttgccatcgtatgatgtggtagatagtggaaaagaatatgatatttacaatattataggtgatcctgaatcgtttaagaaatttcgtatgaaacagcctcctattgcttactggttagaaactaaaaagggaaggaaaggctggttacagaaatatatgcctgctttacctcatggaagcaaacacagggtgtattttaacacaccaaatgggcctcttgaacgagttcctgcgtgggccaattttgtcattccagatgcagacgggatggcattagcagtccattgggaaccacctcctgaatatgcttataaatggaaacacaagctaccagtcaagcctaagtccttgcgcatatatgaatgtcatgttggcatctctggccaggaaccaaaagtttcatctttcaatgattttattagcaaggtccttccgcatgtaaaagaagctggatacaatgcaacgcaaattattggagttgttgagcacaaggattatttcactgttggatatagagtgaccaatttttatgctgttagtagccgttatggcacaccggatgacttcaagcgcttggttgatgaagcacatgggcttggactgcttgtctttttggagattgtgcactcctatgcagcagcagatgaaatggttgggttatctctttttgatggagcaaatgattgctatttccacactggtaaacgtggacaccacaaattctggggcacacggatgttcaaatatggagatcctgatgttctgcactttcttctttcaaatctgaactggtgggtggaggagtatcatgtcgatggcttccattttcattcgctctcgtccatgttgtatacgcataatggatttgcttcatttactggtgacatggatgaatactgtaaccaatatgttgacaaggaggccttattgtacctcatattagcaaatgaagtattacatgctcttcatcctaatgtgatcacgattgctgtggatgcaactctgtatcctggactctgcgatccaacatctcaaggtggactgggctttgattattttgccaatctttctgcctcagagatgtggcttgcattacttgaaaatactcctgatcatgaatggtgcatgagtaagattgttagcacattagtgggcgatagacaaaatactgataaaatgcttttgtatgcagaaaatcacaaccagtccatttctggaggtcgttccttcgcagaaatactgattggtaactccttggggaaatcttccatatcacaagagtcattacttagaggctgctcgttacacaagatgatcagattaattacatctacaattggtggtcatgcatacctcaacttcatgggcaatgaatttggtcacccaaagagagtagagtttccaatgtcaagcaacaatttctccttttcactggctaaccgtcgctgggatctattggaagatgttgtacattatcaattattctcatttgataaggatatgatggacttggataaaaatgggagaattttgtccagaggtcttgccaacattcaccatgtcaatgatactaccatggtgatttcttacttgagaggtcccaatctctttgtgttcaactttcatcctgtcaattcatatgaaagatacattataggtgtggaagaagctggagagtatcaagtcacattaaatacagatgaaaacaagtatggtggtagaggactacttggccatgatcagaatactcaaagaaccattagtagaagagctgatggaatgagattttgcttggaagtacctctgccaagtagaagtgctcaggtctacaagttgacccgaattctaagagcatgatcactctagcaatcaaagtgcctcatatgatcacacaaaagggaaggttctacattgcccttatactgaccaatattgtggcctttccgaggtgagtttctgtgattcttgagcacaggctgttggctagtcagttatcatgaacttttgccttcagcatctggataagcgcttctcctgtgcaatgagggcatggacgaaatttttttggttcgtcatgggagtcaaaagcatctgccagattaagatttcacaggcctcgagtaaaaccatcacttacttaggatacacaaacacatcaacggggtgcaggctctgataccttctaaagtgaagccgaattccagcacactggcggccgttactagtggatccgagctcggtaccaagcttggcg

SEQ ID NO: 4 - protein, 902 aa, Solanum tuberosum
MLSLSDSIRISSPLSDSRLSFLSQTGSRTSRQLKFVRSRRARVSRCRCSATEQPPPQRRKQRPEKYKQSEEEKGIDPVGFLSKYGITHKAFAQFLRERYKSLKDLKDEILTRHFSLKEMSTGYELMGMHRNIQHRVDFLEWAPGARYCALIGDFNGWSTTGNCAREGHFGHDDYGYWFIILEDKLREGEEPDKLYFQQYNYAEDYDKGDTGITVEEIFKKANDEYWEPGEDRFIKSRYEVAAKLYEEMFGPNGPQTEEELEAMPDAATRYKTWKEQQKKDPASNLPSYDVVDSGKEYDIYNIIGDPESFKKFRMKQPPIAYWLETKKGRKGWLQKYMPALPHGSKHRVYFNTPNGPLERVPAWANFVIPDADGMALAVHWEPPPEYAYKWKHKLPVKPKSLRIYECHVGISGQEPKVSSFNDFISKVLPHVKEAGYNATQIIGVVEHKDYFTVGYRVTNFYAVSSRYGTPDDFKRLVDEAHGLGLLVFLEIVHSYAAADEMVGLSLFDGANDCYFHTGKRGHHKFWGTRMFKYGDPDVLHFLLSNLNWWVEEYHVDGFHFHSLSSMLYTHNGFASFTGDMDEYCNQYVDKEALLYLILANEVLHALHPNVITIAVDATLYPGLCDPTSQGGLGFDYFANLSASEMWLALLENTPDHEWCMSKIVSTLVGDRQNTDKMLLYAENHNQSISGGRSFAEILIGNSLGKSSISQESLLRGCSLHKMIRLITSTIGGHAYLNFMGNEFGHPKRVEFPMSSNNFSFSLANRRWDLLEDVVHYQLFSFDKDMMDLDKNGRILSRGLANIHHVNDTTMVISYLRGPNLFVFNFHPVNSYERYIIGVEEAGEYQVTLNTDENKYGGRGLLGHDQNTQRTISRRADGMRFCLEVPLPSRSAQVYKLTRILRA

SEQ ID NO: 5 - DNA, 3047 bp, Solanum tuberosum
  CDS (5)..(2710)
aacaatgctctctctgtcggattcaattcgaatttcttcaccattgagcgattctcgtcttagttttctatctcaaaccggaagcagaaccagtcgccagcttaaatttgttcgcagccgccgggctcgagtttcgaggtgtagatgctcagcaacggagcaaccgccaccgcaacgacggaagcaacgaccggagaagtacaaacagtcggaggaagggaaaggaatcgatcctgttggatttctcagcaaatacggcattactcataaagcgtttgctcaatttcttcgtgaaagatataaatcattgaaggacttgaaggatgaaatattgactcgtcatttcagtctcaaggagatgtctactgggtatgaattaatgggtatgcatcgcaacatacaacatcgagtggatttcttggaatgggctccaggtgctcgctactgtgctctgattggtgacttcaatgggtggtcaacaactggtaactgtgccagagagggtcattttggtcatgacgattatgggtattggtttattattcttgaagataaattacgtgaaggagaagaacctgataaattgtattttcaacagtacaattatgcggaggactatggtaaaggtgacacgggtattaccgtcgaggaaatctttaaaaaagcaaatgatgagtattgggaacctggagaagatcgcttcattaaatcacgttatgaggtggcagcaaagttatatgaggaaatgttcggaccaaatggacctcaaacagaagaggaactagaagcaatgcctgatgcagctacacgatacaaaacttggaaagagcaacaaaaagaggatccggcaagcaatttgccatcgtatgatgtggtagatagtggaaaagaatatgatatttacaatattataggtgatcctgaatcgtttaagaaatttcgtatgaaacagcctcctattgcttactggttagaaactaaaaagggaaggaaaggctggttacagaaatatatgcctgctttacctcatggaagcaaatacagggtgtattttaacacaccaaatgggcctcttgaacgagttcctgcgtgggccaattttgtcattccagatgcaggcgggatggcattagcagtccattgggaaccacctcctgaatatgcttataaatggaaacacaagctaccagtcaagcctaagtccttgcgcatatatgaatgtcatgttggcatctctggccaggaaccaaaagtttcatctttcaatgattttattagcaaggtccttccgcatgtaaaagaagctggatacaatgcaatacaaattattggagttgttgagcacaaggattatttcactgttggatatagagtgaccaatttttatgctgttagtagccgttatggcacaccggatgacttcaagcgcttggttgatgaagcacatgggcttggactgcttgtctttttggagattgtgcactcttatgcagcagcagatgaaatggttgggttatctctttttgatggagcaaatgattgctatttccacactggtaaacgtggacaccacaaattctggggcacacggatgttcaaatatggagatcttgatgttctgcactttcttctttcaaatctgaactggtgggtggaggagtatcatgtcgatggcttccattttcattcgctctcgtccatgttgtatacgcataatggatttgcttcatttactggtgacatggatgaatactgtaaccaatatgttgacaaggaggccttattgtacctcatattagcaaatgaagtattacatgctcttcatcctaatgtgatcacgattgctgaggatgcaactctgtatcctggactctgcgatccaacatctcaaggtggactgggctttgattattttgccaatctttctgcctcagagatgtggcttgcattacttgaaaatactcctgatcatgaatggtgcatgagtaagattgttagcacattagtgggcgatagacaaaatactgataaaatgcttttgtatgcagaaaatcacaaccagtccatttctggaggtcgttccttcgcagaaatactgattggtaactccttggggaaatcctccatatcacaagagtcattacttagaggctgctcgttacacaagatgatcagattaattacatctacaattggtggtcatgcatacctcaacttcatgggcaatgaatttggtcacccaaagagagtagagtttccaatgtcaagcaacaatttctccttttcactggctaaccgtcgctgggatctattggaagatgttgtacattatcaattgttctcatttgataagggtatgatggacttggataaaaatgggagaattttgtccagaggtcttgccaacattcaccatgtcaatgatactaccatggtgatttcttacttgagaggtcccaatctctttgtgttcaactttcatcctgtcaattcatatgaaagatacattataggtgtggaagaagctggagagtatcaagtcacattaaatacagatgaaaacaagtatggtggtagaggactacttggccatgatcagaatattcaaagaaccattagtagaagagctgatggaatgagattttgcttggaagtgcctctgccaagtagaagtgctcaggtctacaagttgacccgaattctaagagcatgatcactctagtaatcaaagtgcctcatatgatgacacaaaaggaaaggttctacattgcccttacactgatcaatattgacacctttccgaggtgagtttctgtgattcttgagcagactgttggctagtcaattatcatgaacttttgccttcagcatccggatagtcgcttctcctgtgcaatgagggcatggacgaatttttttttggcttgtcatgggggtcataagcatccgccagattaagatttcacaggcctcgagtaaaaccatcacttactttaaggatacacaaacacaccaacggggtgcaggctctgataccttctaaagtg

SEQ ID NO: 6 - protein, 902 aa, Solanum tuberosum
MLSLSDSIRISSPLSDSRLSFLSQTGSRTSRQLKFVRSRRARVSRCRCSATEQPPPQRRKQRPEKYKQSEEGKGIDPVGFLSKYGITHKAFAQFLRERYKSLKDLKDEILTRHFSLKEMSTGYELMGMHRNIQHRVDFLEWAPGARYCALIGDFNGWSTTGNCAREGHFGHDDYGYWFIILEDKLREGEEPDKLYFQQYNYAEDYGKGDTGITVEEIFKKANDEYWEPGEDRFIKSRYEVAAKLYEEMFGPNGPQTEEELEAMPDAATRYKTWKEQQKEDPASNLPSYDVVDSGKEYDIYNIIGDPESFKKFRMKQPPIAYWLETKKGRKGWLQKYMPALPHGSKYRVYFNTPNGPLERVPAWANFVIPDAGGMALAVHWEPPPEYAYKWKHKLPVKPKSLRIYECHVGISGQEPKVSSFNDFISKVLPHVKEAGYNAIQIIGVVEHKDYFTVGYRVTNFYAVSSRYGTPDDFKRLVDEAHGLGLLVFLEIVHSYAAADEMVGLSLFDGANDCYFHTGKRGHHKFWGTRMFKYGDLDVLHFLLSNLNWWVEEYHVDGFHFHSLSSMLYTHNGFASFTGDMDEYCNQYVDKEALLYLILANEVLHALHPNVITIAEDATLYPGLCDPTSQGGLGFDYFANLSASEMWLALLENTPDHEWCMSKIVSTLVGDRQNTDKMLLYAENHNQSISGGRSFAEILIGNSLGKSSISQESLLRGCSLHKMIRLITSTIGGHAYLNFMGNEFGHPKRVEFPMSSNNFSFSLANRRWDLLEDVVHYQLFSFDKGMMDLDKNGRILSRGLANIHHVNDTTMVISYLRGPNLFVFNFHPVNSYERYIIGVEEAGEYQVTLNTDENKYGGRGLLGHDQNIQRTISRRADGMRFCLEVPLPSRSAQVYKLTRILRA